(19) United States
(12) Patent Application Publication
(10) Pub. No.: US 2025/0109404 A1
(43) Pub. Date: Apr. 3, 2025

(54) CONSTRUCTS AND METHODS FOR CONTROLLING STOMATAL CLOSURE IN PLANTS

(71) Applicants: LIMAGRAIN EUROPE, SAINT BEAUZIRE (FR); COMMISSARIAT A L'ENERGIE ATOMIQUE ET AUX ENERGIES ALTERNATIVES, PARIS (FR)

(72) Inventors: Nathalie LEONHARDT, Aix en Provence (FR); Ludovic MARTIN, Giat (FR); Serge CHIARENZA, Aix en Provence (FR); Helene JACQUET, Vinon sur Verdon (FR); Laurent NUSSAUME, La Tour d'Aigues (FR); Marie JAVELLE, Chappes (FR)

(73) Assignees: LIMAGRAIN EUROPE, SAINT BEAUZIRE (FR); COMMISSARIAT A L'ENERGIE ATOMIQUE ET AUX ENERGIES ALTERNATIVES, PARIS (FR)

(21) Appl. No.: 18/980,645

(22) Filed: Dec. 13, 2024

Related U.S. Application Data
(62) Division of application No. 16/321,644, filed on Jan. 29, 2019, now Pat. No. 12,180,493.

(30) Foreign Application Priority Data
Aug. 5, 2016 (EP) 16306025.4

(51) Int. Cl. C12N 15/82; A01H 1/00; A01H 5/10; A01H 5/12; A01H 6/46; C07K 14/415

(52) U.S. Cl. CPC C12N 15/8261; A01H 1/00; A01H 5/10; A01H 5/12; A01H 6/4684; C07K 14/415

(57) ABSTRACT

The present invention is related to expressing an AHA5 protein in plants, and preferentially a mutated AHA5 protein leading to a constitutive activity of AHA5, to control stomatal closure and improve tolerance to drought conditions and yield in plants.

### CROSS-REFERENCE TO RELATED APPLICATIONS

This application is a divisional of U.S. application Ser. No. 16/321,644, filed on Jan. 29, 2019, which is a U.S. National Stage of International Appln. PCT/EP2017/069796, filed on Aug. 4, 2017, which itself claims the benefit of European Appln. EP 16306025.4 filed on Aug. 5, 2016, the contents of each of which are incorporated herein by reference in their entireties for all purposes.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created on Dec. 9, 2024, is named B385799US D36201_Sequence Listing.xml and is 255,538 bytes in size.

### BACKGROUND OF THE INVENTION

Drought is one of the greatest limitations to crop expansion outside the present-day agricultural areas. It will become increasingly important in regions of the globe where, in the past, the problem was negligible, due to the recognized changes in global climate. Today there is a concern with improving cultural practices and crop genotypes for drought-prone areas.

In plants, the majority of all water loss occurs through pores on the leaf surface, which are called stomata. The size of the stomatal pores in a leaf is variable and controls the rate of diffusion of water vapour out of the plant. In addition to controlling water loss, stomata allow CO₂ to diffuse into the leaf for photosynthesis. Thereby, stomata permanently control the trade-off between carbon uptake and water loss. Regulation of stomatal movements by guard cells in response to environmental stimuli and stress conditions is a primary factor in determining water use efficiency and productivity of crop plants.

In plants, the primary ion pumps, the H⁺-ATPases, are the major protein of the plasma membrane. Using the chemical energy of ATP, plasma membrane H⁺-ATPases extrude protons from cells of plants to generate electrochemical proton gradients. This gradient has a major role in providing the energy for secondary active transport across the plasma membrane. Molecular studies have shown that plant H⁺-ATPases are encoded by a multigene family. Notably, eleven isoforms named from AHA1 to AHA11 have been identified in the Arabidopsis genome (Axelsen et al. 2001). Alignments between members of this family have shown that they share at least 66% protein sequence identity (Axelsen et al., 2001). They share a common structure with a 100-amino acid long C-terminal domain referred as the R (regulatory) or auto-inhibitory domain, comprising a 14-3-3 binding site and a phosphorylation site at the penultimate Thr residue.

An overview of the expression profiles of members of the Arabidopsis plasma membrane H⁺-ATPase (AHA) family can be found in the Genevestigator database (www.genevestigator.org). Two genes, AHA1 and AHA2, are expressed in almost all tissues and organs. Thus, these genes appear to function as housekeeping genes required for ion homeostasis. Relatively

more AHA1 transcripts are found in shoots, whereas AHA2 is predominantly expressed in roots, especially in root hairs. AHA3, AHA4 and AHA11 also show broad expression throughout the plant, but are not expressed to the same degree. Some degree of specialization is seen as AHA4 has high expression in root endodermis in accordance with reporter gene analysis studies (Vitart et al., 2001). Real time RT-PCR analysis has confirmed
that AHA1, AHA2, AHA3 and AHA11 are the major transcripts found in leaves (Alsterfjord et al., 2004). Reporter gene analyses show that the AHA3 promoter is active in leaf phloem companion cells (DeWitt et al., 1991). Expression patterns of AHA6 and AHA9 (predominantly expressed in anthers), AHA7 and AHA8 (almost exclusive expression in pollen) and AHA10 (highest transcript levels in siliques) suggest that the pumps encoded by these genes have more specialized function. Reporter gene analyses have verified the expression of AHA9 in anthers (Houlne et al., 1994) and AHA10 in the endothelium of the developing seed coat (Baxter et al., 2005). AHA5 was found to be predominantly expressed in guard cells (Ueno et al. 2005). In Arabidopsis guard cells, the expression of all 11 H+-ATPase isogenes can be detected, but AHA1, AHA2 and AHA5 are the predominant isoforms (Ueno et al. 2005).

Plant plasma membrane H⁺-ATPases play a central role in different physiological functions including nutrient uptake, intracellular pH regulation and stomatal opening (Haruta et al., 2015). Besides regulation of physiological processes, the plasma membrane proton pumps also play roles in adaptation of plants to changing conditions. Thus, H⁺-ATPases can be mutual elements for resistance mechanisms that are activated in stress conditions.

To determine the function of the different isoforms in planta, single loss-of-function mutants have been obtained in the plant model Arabidopsis for 10 of the 11 genes. However these mutants exhibited no phenotype under normal growth conditions probably due to functional redundancy. For example aha1 and aha2 knock-down mutants grow normally in standard conditions whereas the double homozygous mutant is embryo-lethal (Haruta et al., 2010). This clearly illustrates these two proteins are essential for plant physiology. In addition, using physiological genetic screens, two Arabidopsis ost2 (open stomata2) mutants have been identified and the positional cloning of the OST2 locus revealed its identity with the known AHA1 gene that codes for a plasma membrane H⁺-ATPase (Merlot et al., 2007). Two dominant change-of-function alleles of OST2 were identified as coding for AHA1-_{P68S} and AHA1-_{L169F-G867S} pump variants, respectively. Both mutations disrupt the auto-inhibitory regulation of the AHA1 pump and cause its constitutive activity. Interestingly, this pump deregulation induced pleiotropic effects including the development of spontaneous necrosis on leaves correlated with an increase in the salicylic acid content (SA), an increase of H⁺ excretion (acidification) in roots, but also an aberrant control of stomatal aperture, resulting in abnormally high water loss from leaves.

Several studies demonstrate that substitutions at several residues result in activated H⁺-ATPases including the two substitutions in AHA1 detected in ost2 mutants. Indeed, mutant forms of AHA1 including W₈₇₅L, G₈₆₇L or P₆₈S rise to high-affinity H⁺-ATPase with increase molecular activity (Baunsgaard et al., 1996; Merlot et al., 2007). Using functional assay based on yeast complementation these substitutions were shown to rescue lethal growth defect in yeast mutant RS-72 disrupted in its endogenous plasma membrane PMA1 but not its wild-type counter (Cid et al., 1987; Baunsgaard et al., 1996; Merlot et al., 2007). In addition, Merlot et al. (2007) teaches that AHA1 is involved in the regulation of stomatal aperture in plants and the stomata of two ost2 mutants are more open especially under dark conditions compared to wild type.

There are many different documents describing the mechanisms for controlling the stomatal closure. However, none of them provide a solution to improve drought tolerance.

In EP2995680, inventors overexpressed AHA2 in Arabidopsis thaliana plants under the control of a guard cell specific promoter. They observed that the stomata were more open in the transformed plants, facilitating photosynthesis and possibly improving yield. AHA2 promotes the opening of the stomata so the person skilled in the art would not consider this gene interesting for improving drought tolerance in plants.

WO2014/012145 intends to inhibit stomatal closure by overexpressing AHA1 in order to increase transpiration. The person skilled in the art would not consider this document when trying to improve drought tolerance.

The sequences of AHA5 from Arabidopsis thaliana and Zea mays are listed in the sequence listing of EP1033405, US 20130074202 and US 20090217414. However, there is no indication of a possible use for AHA5.

A protein closed to ZmAHA5 was described in U.S. Pat. No. 8,577,624. This patent deals with a method to identify a potential inhibitor of a type III P-type ATPase, a method to produce such modulator and a computer-assisted method to perform the invention.

There is still a strong interest to develop solutions for controlling the closing of the stomata in order to improve the drought tolerance in plants and the efficient use of water by plants.

### SUMMARY OF THE INVENTION

The invention consists in expressing an AHA5 protein in plants, and preferentially a mutated AHA5 protein leading to a constitutive activity of AHA5, to control stomatal closure and improve tolerance to drought conditions in plants.

The invention is based on the surprising effect that transformation of plants with a nucleic acid encoding an AHA5 protein, and preferentially a mutated AHA5 protein leading to a constitutive activity of AHA5, gives rise to transgenic plants having an increase tolerance to drought stress compared to a corresponding non-transgenic plant.

The invention thus consists in either (i) overexpressing a wild-type AHA5 protein, or (ii) expressing or overexpressing a mutated AHA5 protein leading to a constitutive activity of AHA5, for controlling stomatal closure and improve tolerance to drought conditions in plants. The invention also consists in methods to obtain drought tolerant plants through stomatal closure.

In a first aspect, the present invention is related to a method to control stomatal closure in a plant, said method comprising expressing or overexpressing an AHA5 protein. Said method allows conferring drought tolerance or drought resistance through the control of stomatal closure due to AHA5 protein activity.

Preferably, the expression or overexpression of AHA5 is obtained by overexpression of a nucleic acid encoding a mutated AHA5 which leads to a constitutive activity of AHA5 protein.

In a second aspect, the present invention is related to a method to identify a plant with closed stomata comprising the step of identifying in a population of plants, the plants overexpressing an AHA5 protein or with an AHA5 constitutively active, in particular the plants overexpressing a wild-type AHA5 protein or a constitutively active AHA5 protein.

In another aspect, the present invention is also related to a method of growing plants comprising the step of sowing plant seeds, wherein said plant seeds originate from plants expressing or overexpressing an AHA5 protein, and the step of growing plants from these sowed seeds, wherein the growing phase is made under drought/water deficit stress conditions.

In one other aspect, the present invention is related to a nucleic acid sequence encoding a constitutively active AHA5 protein, preferably with genetic modifications leading to amino acid modifications in either the first transmembrane segment or in the R1 domain in the auto inhibitory C-terminus of AHA5 protein or to the deletion of at least part of the R1 domain in the auto inhibitory C-terminus of AHA5 protein, and more preferably with genetic modifications leading to either amino acid substitution(s) in the first transmembrane segment or in the R1 domain in the auto inhibitory C-terminus of AHA5 protein.

In a further aspect, the present invention is also related to nucleic acid constructs comprising a promoter functional in plants and allowing at least an expression in the guard cells, which is operably linked to a nucleic acid sequence encoding an AHA5 protein according to the invention.

Transgenic plants comprising a nucleic acid construct comprising a promoter functional in plants and allowing at least an expression in the guard cells which is operably linked to a nucleic acid sequence encoding a constitutively active AHA5 protein, or comprising a nucleic acid encoding a wild-type AHA5 protein linked to a promoter which allows at least an expression in guard cells and wherein the promoter is not the endogenous promoter of the nucleic acid encoding said wild-type AHA5 protein, are other aspects of the present invention.

Still another aspect of the present invention provides methods for decreasing water loss in a plant, by expression or overexpression of an AHA5 protein.

In yet another aspect, the present invention provides a method for increasing plant yield under non-stress or stress conditions for water supply or at least maintaining plant yield under drought/water deficit stress conditions said method comprising a step of growing a transgenic plant overexpressing a wild-type AHA5 protein or a constitutively active AHA5 protein under non-stress conditions or stress conditions for water supply or drought/water deficit conditions.

Suitably, the methods for decreasing water loss or for increasing plant yield comprise expression or overexpression of a constitutively active AHA5 protein, preferably by amino acid substitution(s) in the first transmembrane segment or in the R1 domain in the auto inhibitory C-terminus of AHA5 protein.

In all these aspects related to control of stomatal closure and improvement of tolerance to drought conditions in plants the present invention encompasses expression of an AHA5 protein by either (i) overexpressing a wild-type AHA5 protein, or (ii) expressing or overexpressing a mutated AHA5 protein leading to a constitutively active AHA5 protein in plants and methods thereof.

### BRIEF DESCRIPTION OF THE DRAWINGS

FIG. 1 is a schematic representation of Arabidopsis thaliana AHA5 (AtAHA5) wild-type protein (931 amino acids) with its different functional domains (TM=transmembrane domains; R1 domain in the auto-inhibitory C-terminus). Three positions of amino acid substitutions to obtain a constitutively active AHA5 are represented: P50S, G849S, W857L.

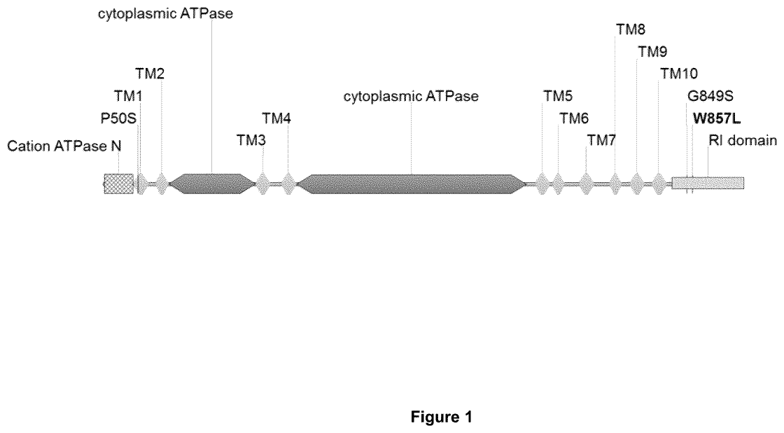

FIG. 2: Complementation of the yeast mutant RS-72 in which the essential gene encoding the H+-ATPase PMA1 was replaced under the strict control of a galactose (GAL)-inducible promoter.

FIG. 3: Expression profile of AtAHA5 gene in plants (Col) transformed with pAHA5: EGFP-GUS construct.

FIG. 4: Expression profile of SbMYB60 gene in plants (Zea mays) transformed with pSbMYB60: ZsGreen construct. Scale bar, 20 μm.

FIG. 5: Water loss experiments in plants transformed with AtAHA5, AtAHA5P50S or AtAHA5W857L under the control of the 35S promoter (OE means overexpression).

FIG. 6: Water loss experiments (A) by day and (B) in kinetics measured in maize plantlets transformed with ZmAHA5 W885L under the control of the pZmRAB17 promoter for three events #A, #B, #C compared to A188 control.

FIG. 7: Measurement of stomatal movements in response to light in plants transformed with AtAHA5, AtAHA5P50S or AtAHA5W857L under the control of the 35S promoter (OE means overexpression).

FIG. 8: Measurement of rosette fresh weight in plants transformed with AtAHA5, AtAHA5P50S or AtAHA5W857L under the control of the 35S promoter (OE means overexpression).

FIG. 9: Relative expression of ZsGreen expressed under the control of the promoter pSbMYB60 in the fourth leaf of transgenic maize plantlets grown under well-watered (WW) or water stress (WS) conditions.

FIG. 10: Relative expression of ZsGreen expressed under the control of the promoter pSbMYB60 in the tenth leaf and the flag leaf of transgenic maize plants grown under well-watered (WW) or water stress (WS) conditions.

FIG. 11: Confocal microscopy imaging of ZsGreen fluorescence in the tenth leaf of transgenic maize plants grown under well-watered (WW) or water stress (WS) conditions. Scale bar, 10 μm.

### DETAILED DESCRIPTION OF THE INVENTION

The present invention is based on the fact that overexpression or expression of an AHA5 protein or a constitutively active AHA5 protein controls stomatal closure in plants, which confers drought tolerance, thus leading to improved, or at least maintained, yield.

According to the present invention, stomatal closure in a plant may be controlled by overexpressing a wild-type AHA5 protein, by overexpressing a mutated AHA5 protein which is constitutively active, or by expressing a constitutively active AHA5 directly in said plant.

In a first aspect, the present invention is related to a method to control stomatal closure in a plant, said method comprising expressing or overexpressing an AHA5 protein, preferably overexpressing an AHA5 protein.

In one embodiment, the method to control stomatal closure in a plant comprises overexpression of an AHA5 protein is a method comprising overexpression of a wild-type AHA5 protein or a constitutively active AHA5 protein. It may be obtained by any conventional methods of overexpression useful in plants such as transformation with a vector comprising said nucleic acid sequence encoding an AHA5 protein. Said transformation may be performed with bacterial strains such as Agrobacterium tumefaciens or by direct methods such as electroporation, gene gun bombardment or other method known by the person skilled in the art. Preferably, the AHA5 protein is overexpressed by transformation of a plant with a vector comprising a promoter functional in plants and a nucleic acid sequence encoding an AHA5 protein, and regenerating said cell to obtain a whole plant. Said vector may be introduced into the plant by Agrobacterium tumefaciens. In particular, it is possible to use the method described by Ishida et al. (1996) for the transformation of Monocotyledons.

In one other embodiment, the method to control stomatal closure in a plant comprises expressing a constitutively active AHA5 protein directly in the plant. It may be obtained by direct mutation of the gene in the plant cell with gene editing techniques, such as CRISPR/Cas9 or TALEN according to the mutations disclosed in the present application. Such techniques are also well known by the skilled person.

The term “stomatal closure” or “closed stomata” as used herein is both referring to a stomata completely closed and to a stomata partially closed. According to the present invention, a stomata is considered closed or partially closed when the aperture between the two guard cells of the stomata is reduced by at least 50% compared to an open stomata in a given plant. Stomata are considered open when the plant is placed under light conditions and non-water stress conditions. In particular, in a preferred embodiment of the present invention, a stomata is considered partially closed when the aperture is reduced by at least 50%, at least 60%, at least 70%, at least 80% and up to 85% compared to an open stomata. Also in a preferred embodiment, the stomata is considered closed when the aperture is reduced by more than 85% compared to an open stomata.

Stomatal closure or aperture may be measured by any classical methods known from the person skilled in the art.

For example, stomatal closure or aperture may be measured by direct methods such as but not limited to visual measures, in particular using microscopy and measuring the aperture between the two guard cells of the stomata under light or dark conditions (Leonhardt N et al.; 1997).

Stomatal closure or aperture may also be measured by indirect methods based on parameters measurements, such as but not limited to:
(i) Rosette leaf adaxial surface temperature, in particular using thermography and for example by infra-red thermography (Merlot S et al., 2002),
(ii) Channel activities, such as potassium or calcium channels, in particular using patch-clamp experiments on guard cells protoplasts (Perfus-Barbeoch L et al., 2002),
(iii) Stomatal conductance, in particular leaf using analysis hand-held porometer which monitors the resistance to gas exchanges, and for example by measuring the leaf temperature and the relative humidity (Clément M et al., 2011), or using gas exchange measurement chambers.
(iv) transpiration rate, in particular based on plant weight loss divided by plant leaf area (Caldeira et al., 2014)

According to a preferred embodiment, the method of controlling stomatal closure comprises expression or overexpression of an AHA5 protein at least in guard cells, and more preferably overexpression of a mutated AHA5 protein which is constitutively active at least in guard cells.

The expression “controlling stomatal closure” or “control of stomatal closure” as used herein means acting on stomatal closure and in particular promoting stomatal closure.

In one embodiment, the method for controlling stomatal closure comprises overexpression a wild-type AHA5 protein in the plant is.

As used herein the term “wild-type” means the amino acid sequence which is a naturally occurring in at least one given organism and which is not changed, modified or mutated by man.

Said wild-type AHA5 protein may be from any plant (genus, species), such as Arabidopsis thaliana (SEQ ID NO: 1), Zea mays (SEQ ID NO: 2), Helianthus annuus (SEQ ID NO: 3 and SEQ ID NO: 4), Lycopersicon esculentum (SEQ ID NO: 5), Aeluropus littoralis (SEQ ID NO: 6 and SEQ ID NO: 7), Sorghum bicolor (SEQ ID NO: 8), Setaria italica (SEQ ID NO: 9), Oryza sativa (SEQ ID NO: 10), Brachypodium distachyon (SEQ ID NO: 11), Hordeum vulgare (SEQ ID NO: 12 and SEQ ID NO: 13), Triticum aestivum (SEQ ID NO: 14 and SEQ ID NO: 15 and SEQ ID NO: 16), Capsella rubella (SEQ ID NO: 17), Camelina sativa (SEQ ID NO: 18), Medicago truncatula (SEQ ID NO: 19), Populus euphratica (SEQ ID NO: 20).

In a preferred embodiment, said wild-type AHA5 protein is selected within the group consisting of the sequence as set forth as SEQ ID NO: 1 and SEQ ID NO: 2.

In another embodiment, and which represents a preferred embodiment, the method for controlling stomatal closure comprises expression or overexpression of a mutated AHA5 which leads to a constitutive activity of the AHA5 protein.

The term “constitutively active AHA5” or “constitutive AHA5 activity” means a mutated AHA5 H+-ATPase so as to be always active. This may be obtained for example by amino acid modifications in either the first transmembrane domain or the R1 regulatory domain of AHA5 protein or by deletion of at least part of the R1 domain in the auto inhibitory C-terminus of AHA5 protein. Preferably the constitutive AHA5 activity may be obtained by either amino acid substitution(s) in the first transmembrane segment or in the R1 regulatory domain of AHA5 protein.

In one embodiment, the method for controlling stomatal closure according to the present invention comprises the expression or overexpression of a constitutively active AHA5 protein which is an AHA5 protein mutated in the first transmembrane domain.

As used herein, the first transmembrane domain consists in the 23 amino acid consensus sequence FLGFMWNPLSWVMEX1AAX2MAIAX3 (SEQ ID NO: 21) wherein:
X₁: M, V, A
X₂: L, I, V
X₃: L, M

Preferably, the method for controlling stomatal closure comprises the expression or overexpression of an AHA5 protein mutated in the first transmembrane domain by substituting the amino acid P at position 8 with the amino acid S with reference to the above consensus sequence (SEQ ID NO: 21) FLGFMWNSLSWVMEX1AAX2MAIAX3 (SEQ ID NO: 22).

In a preferred embodiment, the method for controlling stomatal closure comprises the expression or overexpression of a mutated AHA5 protein from Arabidopsis thaliana as represented by SEQ ID NO: 23.

In one other embodiment, the method for controlling stomatal closure according to the present invention comprises the expression or overexpression of a constitutively active AHA5 protein which is a AHA5 protein modified by amino acid substitution or deletion in the R1 domain.

As used herein, the R1 domain consists in the 107 amino acid consensus sequence

(SEQ ID NOS: 24-27)
LSGX₄AWX₅NX₆LX₇NKX₈AFTX₉KX₁₀X₁₁YGX₁₂X₁₃EREAQWAX₁₄
AQRTLHGLQX₁₅X₁₆EX₁₇X₁₈X₁₉X₂₀X₂₁X₂₂FX₂₃X₂₄KX₂₅SYX₂₆
X₂₇LSX₂₈IAEQAKRRAEX₂₉X₃₀RLREX₃₁X₃₂X₃₃LKX₃₄HVESVVK
LKGLDIX₃₅TIX₃₆QX₃₇YTV,

wherein:
X₄: R, K
X₅: D, L, N, R
X₆: L, M
X₇: E, Q, D
X₈: T, I
X₉: T, S
X₁₀: K, E
X₁₁: D, N
X₁₂: K, R
X₁₃: E, G.
X₁₄: T, A, L
X₁₅: P, A
X₁₆: P, A
X₁₇: T, V, A, P, K, S
X₁₈: no amino acid, A, S, N
X₁₉: no amino acid, S, T, N
X₂₀: N, S, H
X₂₁: N, T
X₂₂: I, V, M, L
X₂₃: N, S, H
X₂₄: E, D
X₂₅: S, N
X₂₆: R, S, H
X₂₇: E, D
X₂₈: E, Q
X₂₉: I, V, M
X₃₀: A, V
X₃₁: L, I, V
X₃₂: N, L, H
X₃₃: T, S
X₃₄: G, S
X₃₅: D, E
X₃₆: Q, N
X₃₇: N, H

Preferably, the method for controlling stomatal closure comprises the expression or overexpression of an AHA5 protein mutated in the R1 domain at one or more, more preferably at one, of the following positions with reference to above consensus sequence SEQ ID NOs: 24-27:
22 wherein the amino acid Y is substituted with A or;
23 wherein the amino acid G is substituted with S or A or;
24 wherein the amino acid K or R is substituted with A or;
27 wherein the amino acid R is substituted with A or;
31 wherein the amino acid W is substituted with L or A or;
32 wherein the amino acid A is substituted with S or;.
35 wherein the amino acid Q is substituted with A or;
36 wherein the amino acid R is substituted with A or;
41 wherein the amino acid L is substituted with A or;
68 wherein the amino acid A is substituted with S or T or;
70 wherein the amino acid R is substituted with A or;
71 wherein the amino acid R is substituted with A or;
74 wherein the amino acid I is substituted with A

Among the above substitutions, one or more, preferably one, of the following positions is preferred:
23 wherein the amino acid G is substituted with S or A or;
31 wherein the amino acid W is substituted with L or A or;
36 wherein the amino acid R is substituted with A or;
41 wherein the amino acid L is substituted with A or;
69 wherein the amino acid R is substituted with A.

The most preferred substitution for obtaining a constitutively active AHA5 protein is at position 31 wherein the amino acid W is substituted with L or A. Therefore, in an advantageous embodiment of the present invention, the method for controlling stomatal closure comprises the expression or overexpression of an AHA5 protein mutated in the R1 domain at position 31 wherein the amino acid W is substituted with L or A with reference to the above consensus sequence SEQ ID NOs: 24-27

(SEQ ID NO: 28)
LSGX₄AWX₅NX₆LX₇NKX₈AFTX₉KX₁₀X₁₁YGX₁₂X₁₃EREAQLAX₁₄
AQRTLHGLQX₁₅X₁₆EX₁₇X₁₈X₁₉X₂₀X₂₁X₂₂FX₂₃X₂₄KX₂₅SYX₂₆
X₂₇LSX₂₈IAEQAKRRAEX₂₉X₃₀RLREX₃₁X₃₂X₃₃LKX₃₄HVESVVK
LKGLDIX₃₅TIX₃₆QX₃₇YTV
or
(SEQ ID NO: 29)
LSGX₄AWX₅NX₆LX₇NKX₈AFTX₉KX₁₀X₁₁YGX₁₂X₁₃EREAQAAX₁₄
AQRTLHGLQX₁₅X₁₆EX₁₇X₁₈X₁₉X₂₀X₂₁X₂₂FX₂₃X₂₄KX₂₅SYX₂₆
X₂₇LSX₂₈IAEQAKRRAEX₂₉X₃₀RLREX₃₁X₃₂X₃₃LKX₃₄HVESVVK
LKGLDIX₃₅TIX₃₆QX₃₇YTV.

In a preferred embodiment, the method for controlling stomatal closure comprises the expression or overexpression of a mutated AHA5 protein from Arabidopsis thaliana as represented by SEQ ID NO: 30 or the expression or overexpression of a mutated AHA5 protein from Zea mays as represented by SEQ ID NO: 31.

In a further embodiment of the present invention, the method for controlling stomatal closure comprises the expression or overexpression of an AHA5 protein lacking at least part of the R1 domain in the auto inhibitory C-terminus.

In this particular embodiment, the R1 domain may be completely deleted, thus lacking the 107 amino acids of sequence SEQ ID NOs: 24-27 at the C-terminus of the AHA5 protein. The R1 domain may also be deleted in part, and preferably of at least the 67 amino acids, preferably of at least the 78 amino acids, preferably of at least the 93 amino acids, preferably of at least the 96 amino acids from the C-terminal end of the AHA5 protein. The above positions are with reference to above-mentioned R1 domain consensus sequence SEQ ID NOs: 24-27.

The method for controlling stomatal closure according to the present invention may be for controlling stomatal closure in any type of plant, such as: monocotyledons like maize, wheat, sorgho, rice, barley, sugarcane, or dicotyledons like sunflower or plants from the Brassicaceae family like Arabidopsis thaliana, rapeseed, Brassica or from the Solanaceae family like tomato and potato.

Preferably, the method is for controlling stomatal closure in monocotyledons, and more preferably selected in the group consisting of maize and wheat and most preferably in maize.

In a specific embodiment, the method of the present invention is for controlling stomatal closure in maize by overexpression of the AHA5 protein from the genus Arabidopsis, in particular Arabidopsis thaliana or by overexpression of the AHA5 protein from Zea mays.

In a second aspect, the present invention is related to a method to identify a plant with closed stomata comprising the step of identifying in a population of plants, the plants overexpressing AHA5 protein or with an AHA5 protein constitutively active, in particular the plants overexpressing a wild-type AHA5 protein or an AHA5 protein constitutively active.

In a specific embodiment, the identification is performed through the use of a marker that is specific to the transgene. In this embodiment, the identification step is thus preferably preceded by a step comprising genotyping said population of plants.

In a specific embodiment, the identification step is preceded by a step comprising extracting the RNA from the individuals in said population.

In a specific embodiment, the identification step is preceded by a step comprising extracting proteins from the individuals in said population. Any classical method known in the art for detecting and quantifying the level of the AHA5 protein may be used, such as Western Blot.

In another aspect, the present invention is also related to a method of growing plants comprising the step of:
sowing plant seeds, wherein said plant seeds originate from plants overexpressing an AHA5 protein,
growing plants from these sowed seeds, wherein the growing phase is made under drought/water deficit stress conditions.

This method may also comprise a step of harvesting said plants.

As used herein, the expressions “drought stress” and “water deficit stress” are synonymous conditions. The term “drought stress” refers to a condition without normal watering in plant growth, which is utilized as a very common term including all kind of abiotic stresses that induce harmful effects on plant growth and survival, for example “drought stress” as used herein includes such stresses as e.g., soil water deficit, vapor pressure deficit, heat stress or light radiation. More specifically, the term “drought” and “water-deficit” refers to environmental conditions where the amount of water (e.g., rainfall or other available water source for plant life) is less than the average water conditions for the particular environment, or the amount of water available is less than the amount of water typically needed by a certain species of plant or by a plant growing in a particular environment.

According to the present application, a drought stressed location is for example a location where the grain yield potential of the site has not been reached due to a drought stress.

A non-stressed location is for example a location where the grain yield potential has been reached by a commercial hybrid variety.

The drought stress intensity may be evaluated by measuring the yield lost between the drought stress treatment (WUE) and a reference treatment irrigated with an optimal amount of water, which is at least, equivalent to the maximum evapotranspiration (ETM) of the crop.

A yield loss of −30% is generally targeted with a common distribution of the drought location between −10% and −40% of yield.

A low drought stressed location may typically be a location with a yield lost between 0% and up to −20% a moderate stressed location between −20% and up to −30%.

The targeted growth stage period may typically be from tasseling to R2 growth stage.

In a common drought location, the drought stress period can spread out from a period between V10 and R4 growth stage.

The terms “drought-resistance” or “drought-tolerance” refer to the ability of a plant to recover from periods of drought stress (i.e., little or no water for a period of days). In the context of the present invention, drought tolerance refers to the ability of a plant to achieve a yield performance as close as possible to the optimal yield whatever the intensity and the duration of the stress.

Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant. The yield may be expressed for example in q/ha (q means quintal which correspond to 100 kg and ha means hectare).

For the present invention, the yield may be calculated as follows:
During harvest, grain weight and grain moisture are measured using on-board equipment on the combine harvester.
Grain weight is then normalized to moisture at 15%, using the following formula:

Normalized grain weight=measured grain weight×(100−measured moisture (as a percentage))/85 (which is 100−normalized moisture at 15%). As an example, if the measured grain moisture is 25%, the normalized grain weight will be: normalized grain weight=measured grain weight×75/85.
Yield is then expressed in a conventional unit (such as quintal per hectare).

In a specific embodiment, the method of growing plants according to the invention comprises the step of sowing plant seeds originate from plants overexpressing a constitutively active AHA5 protein, and preferably overexpressing a constitutively active AHA5 protein as defined above.

In one other aspect, the present invention is related to a nucleic acid sequence encoding a constitutively active AHA5 protein, preferably with genetic modifications leading to amino acid modifications in either the first transmembrane segment as described above as SEQ ID NO: 21, or in the R1 domain in the auto-inhibitory C-terminus of AHA5 protein or by deletion of at least part of the R1 domain in the auto-inhibitory C terminus of AHA5 as described above as SEQ ID Nos: 24-27, and more preferably leading to either amino acid substitution(s) in said first transmembrane segment or in said R1 domain in the auto inhibitory C-terminus of AHA5 protein.

In a preferred embodiment, the nucleic acid sequence encodes a constitutively active AHA5 protein mutated in the first transmembrane domain and in a more preferred embodiment the AHA5 protein is mutated in the first transmembrane domain by substituting the amino acid P at position 8 with the amino acid S with reference to the above consensus sequence (SEQ ID NO: 21).

In a most preferred embodiment, the nucleic acid sequence encodes a constitutively active AHA5 protein from Arabidopsis thaliana, said protein being represented by SEQ ID NO: 23.

In one other preferred embodiment, the nucleic acid sequence encodes a constitutively active AHA5 protein modified by amino acid substitution or deletion in the R1 domain. More preferably, said AHA5 protein is mutated in the R1 domain at one or more, and preferentially one, of the following positions with reference to consensus sequence SEQ ID NOs: 24-27:
22 wherein the amino acid Y is substituted with A or;
23 wherein the amino acid G is substituted with S or A or;
24 wherein the amino acid K or R is substituted with A or;
27 wherein the amino acid R is substituted with A or;
31 wherein the amino acid W is substituted with L or A or;
32 wherein the amino acid A is substituted with S or;
35 wherein the amino acid Q is substituted with A or;
36 wherein the amino acid R is substituted with A or;
41 wherein the amino acid L is substituted with A or;
68 wherein the amino acid A is substituted with S or T or;
70 wherein the amino acid R is substituted with A or;
71 wherein the amino acid R is substituted with A or;
74 wherein the amino acid I is substituted with A.

In an even more preferred embodiment, the nucleic acid sequence encodes a constitutively active AHA5 protein mutated in the R1 domain at one or more, preferentially one, of the following positions with reference to consensus sequence SEQ ID NOs: 24-27:
23 wherein the amino acid G is substituted with S or A or;
31 wherein the amino acid W is substituted with L or A or;
36 wherein the amino acid R is substituted with A or;
41 wherein the amino acid L is substituted with A or;
69 wherein the amino acid R is substituted with A.

In the most preferred embodiment, the nucleic acid sequence encodes a constitutively active AHA5 protein mutated in the R1 domain at position 31 wherein the amino acid W is substituted with L or A (SEQ ID NO: 28 and SEQ ID NO: 29, respectively).

In a preferred embodiment, the nucleic acid sequence encodes a mutated AHA5 protein from Arabidopsis thaliana, said protein being represented by SEQ ID NO: 30 or the nucleic acid encodes a mutated AHA5 protein from Zea mays, said protein being represented by SEQ ID NO: 31.

In another preferred embodiment, the nucleic acid sequence encodes an AHA5 protein lacking at least part of the R1 domain in the auto inhibitory C-terminus.

In a further aspect, the present invention is also related to nucleic acid constructs comprising a promoter functional in plants and allowing at least an expression in the guard cells, which is operably linked to a nucleic acid sequence encoding a constitutively active AHA5 protein as defined above.

A promoter functional in plants refers to a nucleotide sequence capable of initiating transcription of a nucleic acid molecule in a cell of a plant. A promoter functional in plants can be for example a constitutive promoter, an inducible promoter, a developmentally regulated promoter or a tissue-specific promoter such as a leaf-specific promoter, a seed-specific, a guard cell-specific promoter and the like.

According to the present invention, the nucleic acid constructs comprise a promoter functional in plants and allowing at least an expression in the guard cells of the stomata.

The nucleic acid constructs of the present invention may comprise a promoter which is specific of the guard cells such as the SbMYB60 or a promoter allowing expression in the whole plant such as the CsvMV promoter, or a promoter driving expression in leaf such as the rbcs promoter or a drought inducible promoter such as RAB17.

In another aspect the present invention is also related to nucleic acid constructs comprising a nucleic acid encoding a wild-type AHA5 protein linked to promoter which is not the endogenous promoter of the nucleic acid encoding said wild-type AHA5 protein and which allows at least an expression in the guard cells.

All the nucleic acid constructs of the present invention, either containing a nucleic acid sequence encoding a constitutively active AHA5 protein with an endogenous or non-endogenous promoter or containing a nucleic acid sequence encoding a wild-type AHA5 protein under the control of a non-endogenous promoter, are stomata closure-controlling constructs.

A further aspect of the invention consists in transgenic plants comprising a nucleic acid construct comprising a promoter functional in plants operably linked to a nucleic acid sequence encoding a constitutively active AHA5 protein or transgenic plants comprising a nucleic acid construct comprising a promoter operably linked to a nucleic acid sequence encoding a wild-type AHA5 protein, said promoter being non-endogenous as described above.

All the above preferred embodiments concerning the nucleic acid sequence encoding a constitutively active AHA5 protein also apply to the nucleic acid constructs according to the invention.

Still another aspect of the present invention provides methods for decreasing water loss in a plant, by expressing or overexpression of an AHA5 protein in said plant, in particular by overexpressing a wild-type AHA5 protein or by expressing or overexpressing a constitutively active AHA5 protein in said plant.

In yet another aspect, the present invention provides a method for increasing plant yield under non-stress or stress conditions for water supply or at least maintaining plant yield under drought/water deficit stress conditions said method comprising a step of growing a transgenic plant overexpressing a wild-type AHA5 protein or a constitutively active AHA5 protein under non-stress conditions or stress conditions for water supply or under drought/water deficit conditions.

As used herein, the expression “non-stress conditions for water supply” means a condition with normal or sufficient water supply with respect to the species of plant and to the particular environment wherein the plant is growing.

All the preferred embodiments described herein for expression or overexpression of AHA5 protein for controlling stomatal closure in a plant apply mutatis mutandis for expression or overexpression of AHA5 protein in the methods according to the invention for decreasing water loss in a plant or for increasing plant yield.

Suitably, the methods for decreasing water loss or for increasing plant yield comprise expression or overexpression of a constitutively active AHA5 protein, which is preferably an AHA5 mutated by amino acid modifications in either the first transmembrane or the R1 regulatory domain of AHA5 protein or by deletion of at least part of the R1 domain in the auto inhibitory C-terminus of AHA5 protein. More preferably, the constitutively active AHA5 protein is an AHA5 mutated by amino acid substitution(s) in the first transmembrane segment or in the R1 regulatory domain of AHA5 protein.

According to the present invention, the increasing of plant yield is wherein:
the yield obtained from said transgenic plant grown under said water deficit stress conditions is increased as compared to the yield obtained from a plant not overexpressing an AHA5 protein, grown under said stress conditions, or
the yield obtained from said transgenic plants grown under non-stress conditions for water supply is increased as compared to the yield obtained from a plant not overexpressing an AHA5 protein grown under said non-stress conditions, or
the yield obtained from said transgenic plants grown under said water deficit stress conditions is maintained as compared to the yield obtained from said transgenic plant grown in non-stress conditions for water supply.

In yet another aspect, the present invention provides a promoter of sequence with at least 80%, preferably with at least 85%, more preferably with at least 90%, and even more preferably with at least 95% sequence identity with SEQ ID NO: 40. This promoter is able to drive expression specifically in the guard cells of a plant. This promoter is also able to be induced by drought stress, viz. is a drought stress-inducible promoter. The identity percentage is calculated by any sequence analysis method known by the skilled person, and particularly with algorithms such as Needleman-Wunsch. The identity percentage is calculated over the whole length of the query sequence.

In a preferred embodiment, the promoter is SEQ ID NO: 40.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The following examples illustrate certain aspects of the invention. The examples in no way limit the invention.

### EXAMPLES

### Example 1

### Identification of Two Mutations Involved in the Activity Regulation of Plasma Membrane Proton Pumps H⁺-ATPases

The AtAHA5 gene was synthetized by the gene synthesis provider GeneArt and cloned into the pMK vector. Subsequently, AtAHA5 was amplified by PCR using the following primers: 5′-GGATTCTAGAACTAGTATGGAGGAAGTGTTCGAGGAGCTG-3′ (SEQ ID NO: 32) and 5′-CGGTATCATAAGCTTGTTAAACGGTGTAATGTTGCTGAATCG-3′ (SEQ ID NO: 33) and cloned into the pAG425GPD excised by Spel and Hindlll using In-Fusion strategy (Clontech), following the manufacturer's instructions. Then, to test substitutions previously identified in AHA1, AtAHA5 variants were generated by introducing point mutations into AtAHA5. In AHA1, these mutations give rise to high-affinity H⁺-ATPase with increase molecular activity.

Single amino acid substitution, P₅₀S or W₈₅₇L, was introduced into AtAHA5 by site-directed mutagenesis using Site-Directed Mutagenesis kit (Agilent technology), following the manufacturer's instructions and using the following primers: 5′-GGGTTCATGTGGAACTCATTGTCGTGG-3′ (SEQ ID NO: 34) and 5′-CCACGACAATGAGTTCCACATGAACCC-3′ (SEQ ID NO: 35) or 5′-GGAAAGAGAAGCTCAATTGGCTGCAGCTC-3′ (SEQ ID NO: 36) and 5′-GAGCTGCAGCCAATTGAGCTTCTCTTTCC-3′ (SEQ ID NO: 37), respectively.

To evaluate the impact of these substitutions, functional assay based on yeast complementation was performed using the yeast mutant RS-72 disrupted in its endogenous plasma membrane ATPase. In the RS-72 yeast strain, the native promoter of the yeast H⁺-ATPase (PMA1) has been replaced by a galactose-dependent (GAL1) promoter. Expression of plasmid-born plant H+-ATPase is under the control of the constitutive PMA1 promoter. Thus, when the yeasts are maintained on galactose medium, both the yeast and the plant H⁺-ATPases are expressed. After transfer to glucose medium only plant H⁺-ATPase is being produced and, in order to grow, yeasts are dependent on the plant enzyme (Cid et al., 1987).

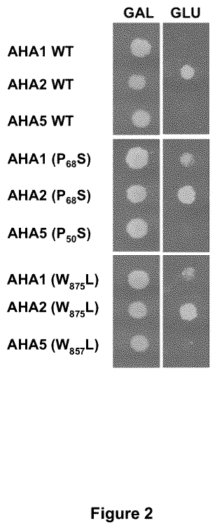

Yeast expressing wild-type AtAHA5 but no endogenous yeast ATPase do not grow at all on glucose medium as previously observe for AHA1 (FIG. 2). Surprisingly, none of the amino acid substitution, P₅₀S or W₈₅₇L is able to rescue the growth phenotype of the yeast compare to AHA1 or AHA2. Using Western analysis with antibodies directed against the plant H⁺-ATPases on membrane proteins from yeast, the expression of AtAHA5 protein in yeast was confirmed (Data not shown).

These results show that amino acid substitutions P₆₈S and W₈₇₅L in AHA1 and AHA2 proteins, but not the corresponding amino acid substitutions P₅₀S and W₈₅₇L in AHA5, are able to complement the growth defect of RS-72.

These variants were then used to transform Arabidopsis thaliana plants to evaluate the impact of these proteins in stomatal closure and water loss rate coupled to biomass evaluation in response to drought stress.

### Example 2

### Guard Cell Specific Expression of the Marker Gene GUS Under Control of AtAHA5 Promoter Region

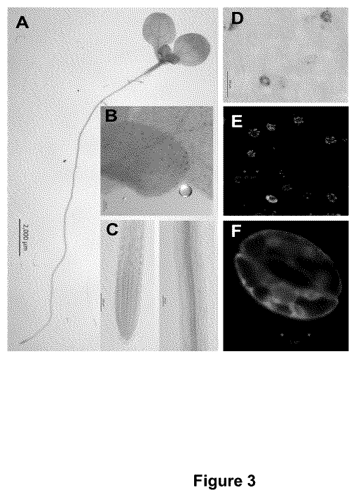

Previous studies based on RT-PCR indicate that AtAHA5 is expressed in stomata (Ueno et al., 2005, Leonhardt et al, 1999). To determine the AtAHA5 expression profile in detail, transgenic plants expressing the reporter β-glucuronidase (GUS) gene under the control of the putative AtAHA5 promoter were generated. The genomic region located upstream of the translation initiation codon, 2356bp in length, was amplified by PCR on genomic DNA from Arabidopsis Columbia ecotype (Col) using the primers 5′-GGG GAC AAC TTT GTA TAG AAA AGT TGC AAC CAT CAT GAC AAG CGT CTG-3′ (SEQ ID NO: 38) and 5′-GGG GAC TGC TTT TTT GTA CAA ACT TGG GGT ATT CGT ACC TAG ACC CAT CAT T-3′ (SEQ ID NO: 39). This 2356bp genomic fragment was then cloned upstream the EGFP (Enhance Green Fluorescence Protein) and GUS (β-glucuronidase) reporter genes in the pBGWFS7 vector. The construct obtained was introduced in Arabidopsis and the resulting transgenic lines were histologically analyzed to detect the reporter expression domains. In all samples examined, reporter gene expression was only detected in guard cells from all plant aerial organs provided with stomata (FIG. 3).

These results show that GUS and GFP were detected only in guard cell (B, D, E, F). No GUS activity or EGFP were detected in roots (A, B, C) or in vascular tissues and mesophyll in leaves (A).

### Example 3

### Identification of Quard-Cell Promoters SbMYB60 and ZmMYB60

Using AtMYB60 protein sequence, first hits by blastp against the Arabidopsis genome (TAIR_V9), rice genome (MSU Rice Genome Release 7), maize genome (B73 RefGen_v2), and sorghum genome (MIPS 1.4) were selected and aligned using Muscle module of seaview4 (Gouy M. et al., (2010). Conserved blocks were identified and provided to the phyML 3.0 software (Guindon S. et al., 2010). Closest homologous genes were identified in rice (LOC_Os11g35390), sorgho (Sb05g021820) and maize (GRMZM2G108959). The upstream region up to the next repetitive region were isolated and aligned to identify conserved blocks and cis-elements. From a region of 922 bp upstream of Sb05g021820, one ABRE box was identified 149 bp upstream of the putative start codon. The ABRE box was mutated from ACGTG in order to create a DOF element AAAAG. An additional base was modified 682 bp upstream of the putative start codon for cloning purposes. Similarly in maize from a region of 773 bp upstream of the GRMZM2G108959 gene, one ABRE box was identified 71 bp upstream of the putative start codon. The ABRE box was mutated from ACGTG in order to create a DOF element AAAAG. An additional base was modified 702 bp upstream of the putative start codon for cloning purposes.

The regulatory region upstream of the putative start codon of SbMYB60 as exemplified above (SEQ ID NO: 40) was cloned and fused to the reporter gene ZsGreen (Clonetech; Matz et al., 1999) and the Arabidopsis thaliana Sac66 polyadenylation sequence, and transformed into maize. Guard cell specific expression of the ZsGreen fluorescence protein was verified by confocal microscopy of primary transformants seedlings. The guard cell specific Sorghum bicolor MYB60 promoter allows strong expression in guard cells. The Sorghum bicolor gene was identified as the closest homologue of AtMYB60 (Rusconi et al., 2013).

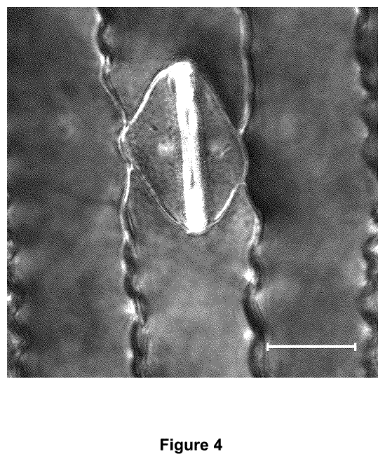

The maize T0 plantlets stably transformed with the fusion proSMYB60: ZsGreen were analyzed by confocal microscopy. As shown in FIG. 4, fluorescence of the ZsGreen marker gene was detected in guard cells but not subsidiary cells neither in epidermal pavement cells of maize leaves.

The maize MYB60 promoter was also identified (SEQ ID NO: 41) as described above.

### Example 4

### Cloning of AtAHA5 Downstream the 35S Promoter and Downstream the AtAHA5 Promoter and Transformation of Arabidopsis Plants

The AtAHA5 gene was synthetized by the gene synthesis provider GeneArt and cloned into the pMK vector. AtAHA5 was subsequently excised by Ascl, Pacl cleavage and cloned in the Ascl, Pacl sites of the gateway constitutive expression vector, pMDC32, harboring a dual 35S promoter (SEQ ID NO: 42). Then, single amino acid substitution, P₅₀S or W₈₅₇L, was introduced into pMDC32 by site-directed mutagenesis using the following primers: 5′-GGGTTCATGTGGAACTCATTGTCGTGG-3′ (SEQ ID NO: 34) and 5′-CCACGACAATGAGTTCCACATGAACCC-3′ (SEQ ID NO: 35) or 5′-GGAAAGAGAAGCTCAATTGGCTGCAGCTC-3′ (SEQ ID NO: 36) and 5′-GAGCTGCAGCCAATTGAGCTTCTCTTTCC-3′ (SEQ ID NO: 37), respectively.

The p35S: AtAHA5P₅₀S construct corresponds to SEQ ID NO: 43 and the p35S: AtAHA5W₈₅₇L construct corresponds to SEQ ID NO: 44.

The AtAHA5 wild-type gene was also cloned downstream of the dual 35S promoter to make the construct SEQ ID NO: 45.

The promoter of AtAHA5 (SEQ ID NO: 46) was fused to the EGFP and the AtAHA5 gene into the pB7m34G vector using the gateway cloning strategy. Then, single amino acid substitution, W₈₅₇L, was introduced into pB7m34G by site-directed mutagenesis using the following primers: 5′-GGAAAGAGAAGCTCAATTGGCTGCAGCTC-3′ (SEQ ID NO: 36) and 5′-GAGCTGCAGCCAATTGAGCTTCTCTTTCC-3′ (SEQ ID NO: 37). The construct (SEQ ID NO: 47) was introduced in Arabidopsis.

The AtAHA5 wild-type gene was also cloned downstream of the promoter of AtAHA5 (SEQ ID NO: 48).

All of the plant transformation plasmid vectors were introduced into Agrobacterium tumefaciens, which was then used to transform Arabidopsis plants belonging to the Columbia ecotype or ost2-2D mutant using a standard method (Weigel and Glazebrook. 2006). F3 homozygous plants were used for the experiments.

### Example 5

Cloning of the Mutated ZmAHA5 Downstream of CsVMV, rbcs, ZmRAB17 and SbMYB60 Promoters and Transformation in Corn

The ZmAHA5 coding sequence (GRMZM2G006894) with the mutation W885L (SEQ ID NO: 49 or SEQ ID NO: 50 corresponding to two optimized versions with respect to allergenicity issue) was codon optimized for maize expression by a gene synthesis service provider and cloned into the pUC57 vector (Genscript). The mutated ZmAHA5 sequence was then cloned via restriction enzyme digestion and ligation between a CsVMV promoter (Verdaguer et al (1996)) (SEQ ID NO: 51) plus a rice actin intron (McElroy et al 1990) (SEQ ID NO: 52), and an Arabidopsis Sac66 polyadenylation sequence (Jenkins et al., 1999) (SEQ ID NO: 53), into the destination binary plasmid pBIOS03092 forming pBIOS03580. (The destination vector is a derivative of pSB12 (Komari et al. (1996)) containing a pActin+actin intron-selectable marker-nos polyA chimeric gene for selection of maize transformants and cassette between a HMWG promoter, the reporter gene ZsGreen (Clonetech; Matz et al., 1999) and a Sac66 polyadenylation sequence).

Similarly, the mutated ZmAHA5 was linked to the Rbcs promoter (Matsuoka and Sanada, 1991) (SEQ ID NO: 54) and a Zea mays Rbcs polyadenylation sequence (SEQ ID NO: 55), by performing a restriction enzyme digestion and ligation and the destination binary plasmid pBIOS03092 forming pBIOS03583.

Similarly, the mutated ZmAHA5 was linked to the drought inducible Zea mays Rab17 promoter (Vilardell et al., 1991) (SEQ ID NO: 56) and a Zea mays Rab17 polyadenylation sequence (SEQ ID NO: 57), by performing a restriction enzyme digestion and ligation and the destination binary plasmid pBIOS03092 forming pBIOS10481.

The mutated ZmAHA5 was linked to the guard cell specific Sorghum bicolor MYB60 promoter (SEQ ID NO: 40) and an Arabidopsis thaliana Sac66 polyadenylation sequence (SEQ ID NO: 53), by performing a restriction enzyme digestion and ligation and the destination binary plasmid pBIOS03092 forming pBIOS10646.

pBIOS03580, pBIOS03583, pBIOS10481, and pBIOS10646 were transferred into agrobacteria LBA4404_+pSB1 according to Komari et al (1996) forming respectively strain T02781, strain T02784, strain T10401 and strain T10584. Maize cultivar A188 was transformed with these agrobacterial strains essentially as described by Ishida et al (1996).

Analysis of the pCsVMV-ZmAHA5_{W885L}-AtSac66 term (SEQ ID NO: 58), pRbcs-ZmAHA5_{W885L}-Rbcs term (SEQ ID NO: 59), pZmRAB17-ZmAHA5_{W885L}-ZmRab17 term (SEQ ID NO: 60), and pSbMYB60-ZmAHA5_{W885L}-AtSac66 term (SEQ ID NO: 61) transformed maize plants indicated that plants overexpressed the mutated ZmAHA5. Overexpression of the transgene was verified by quantitative reverse-transcription polymerase chain reaction (q-RT-PCR). Total RNA was extracted from T0 plantlets leaf. RNA was reverse transcribed into cDNA; mutated ZmAHA5 expression was quantified on those cDNA and normalized with endogenous ZmEF1□□ (GRMZM2G001327) and ZmUbiquitin (GRMZM2G110983) genes expression.

### Example 6

Cloning of the Mutated AtAHA5 Downstream of CsVMV, rbcs, ZmRAB17 and SbMYB60 Promoters and Transformation in Corn

The AtAHA5 coding sequence (AT2G24520) with the mutation W857L (SEQ ID NO: 62) was codon optimized for maize expression by a gene synthesis service provider and cloned into the pUC57 vector (Genscript). The AtAHA5 sequence was then cloned via restriction enzyme digestion and ligation between a CsVMV promoter (Verdaguer et al (1996)) (SEQ ID NO: 51) plus a rice actin intron (McElroy et al 1990) (SEQ ID NO: 52), and an Arabidopsis Sac66 polyadenylation sequence (Jenkins et al (1999)) (SEQ ID NO: 53), into the destination binary plasmid pBIOS3091 forming pBIOS03579.

Similarly, the mutated AtAHA5 was linked to the rbcs promoter (SEQ ID NO: 54) by performing restriction enzyme digestion and ligation between a rbcs promoter, and a Zea mays rbcs polyadenylation sequence (SEQ ID NO: 55), into the destination binary plasmid pBIOS03092 forming pBIOS03582.

Similarly, the mutated AtAHA5 was linked to the drought inducible Zea mays Rab17 promoter (Vilardell et al., 1991) (SEQ ID NO: 56) and a Zea mays Rab17 polyadenylation sequence (SEQ ID NO: 57), by performing a restriction enzyme digestion and ligation and the destination binary plasmid pBIOS03092 forming pBIOS10231.

The mutated AtAHA5 was linked to the guard cell specific Sorghum bicolor MYB60 promoter (SEQ ID NO: 40) and an Arabidopsis thaliana Sac66 polyadenylation sequence (SEQ ID NO: 53), by performing a restriction enzyme digestion and ligation and the destination binary plasmid pBIOS03092 forming pBIOS10648.

pBIOS03579, pBIOS03582, pBIOS10231, and pBIOS10648 were transferred into agrobacteria LBA4404 (pSB1) forming respectively strain T02780, strain T02783, strain T10191 and strain T10586. Maize cultivar A188 was transformed with these agrobacterial strains essentially as described by Ishida et al (1996).

Analysis of the pCsVMV-AtAHA5W857L-AtSac66 term (SEQ ID NO: 63), pRbcs-AtAHA5 W857L-ZmRbcs term (SEQ ID NO: 64), pZmRAB17-AtAHA5W857L-ZmRAB17 term (SEQ ID NO: 65), and pSbMYB60-AtAHA5W857L-AtSac66 term (SEQ ID NO: 66) transformed maize plants indicated that plants overexpressed the mutated AtAHA5. Overexpression of the transgene was verified by quantitative reverse-transcription polymerase chain reaction (q-RT-PCR) as previously described.

### Example 7

### Maize Field Trials

### A—Field Trials

Hybrids were obtained from a cross of T3 plants issued from the AHA5transgenic maize line (pCsVMV-ZmAHA5W885L-AtSac66 term (SEQ ID NO: 58), pRbcs-ZmAHA5W885L-Rbcs term (SEQ ID NO: 59), pZmRAB17-ZmAHA5 W885L-ZmRab17 term (SEQ ID NO: 60), pSbMYB60-ZmAHA5W885L-AtSac66 term (SEQ ID NO: 61), pCsVMV-AtAHA5W857L-AtSac66 term (SEQ ID NO: 63), pRbcs-AtAHA5 W857L-ZmRbcs term (SEQ ID NO: 64), pZmRAB17-AtAHA5W857L-ZmRAB17 term (SEQ ID NO: 65), and pSbMYB60-AtAHA5W857L-AtSac66 term (SEQ ID NO: 66)) chosen according to the previous example with a tester line.

The transformant (T0) plant was first crossed with the A188 line thereby producing T1 plants. T1 plants were then self-pollinated twice, producing T3 plants which are homozygous lines containing the transgene. These T3 plants were then crossed with the tester line thereby leading to a hybrid. This hybrid is at a T4 level with regards to the transformation step and is heterozygous for the transgene. These hybrid plants are used in field experiments.

Control hybrids are obtained as follows:

Control Equivalent corresponds to a cross between a A188 line (the line used for transformation) and the tester line.

Yield was calculated as follows:

During harvest, grain weight and grain moisture are measured using on-board equipment on the combine harvester.

Grain weight is then normalized to moisture at 15%, using the following formula:

Normalized grain weight=measured grain weight×(100−measured moisture (as a percentage))/85 (which is 100−normalized moisture at 15%).

As an example, if the measured grain moisture is 25%, the normalized grain weight will be: normalized grain weight=measured grain weight×75/85.

Yield is then expressed in a conventional unit (such as quintal per hectare).

### B—Experimental Design

Field trials are on 3 different locations.

The experimental block comprises 4 replicates. The experimental design was Randomized Lattice blocks in drought stressed locations. Each replicate comprised of two row plots with about up to 70 plants per plot at a density of 75 000 plants/ha.

Controls were used present in this experiment as described above a control equivalent (A188 crossed with the tester line).

A drought stressed location is a location where the grain yield potential of the site has not been reached due to a drought stress.

A non-stressed location is a location where the grain yield potential has been reached by a commercial hybrid variety.

The drought stress intensity is evaluated by measuring the yield lost between the drought stress treatment (WUE) and a reference treatment irrigated with an optimal amount of water, which is at least, equivalent to the maximum evapotranspiration (ETM) of the crop.

A yield loss of −30% is targeted with a common distribution of the drought location between −10% and −40% of yield.

A low drought stressed location is typically a location with a yield lost between 0% and up to −20% a moderate stressed location between −20% and up to −30%.

The targeted growth stage period is typically from tasseling to R2 growth stage.

In a common drought location, the drought stress period can spread out from a period between V10 and R4 growth stage.

### Example 8

### Water Loss Experiments

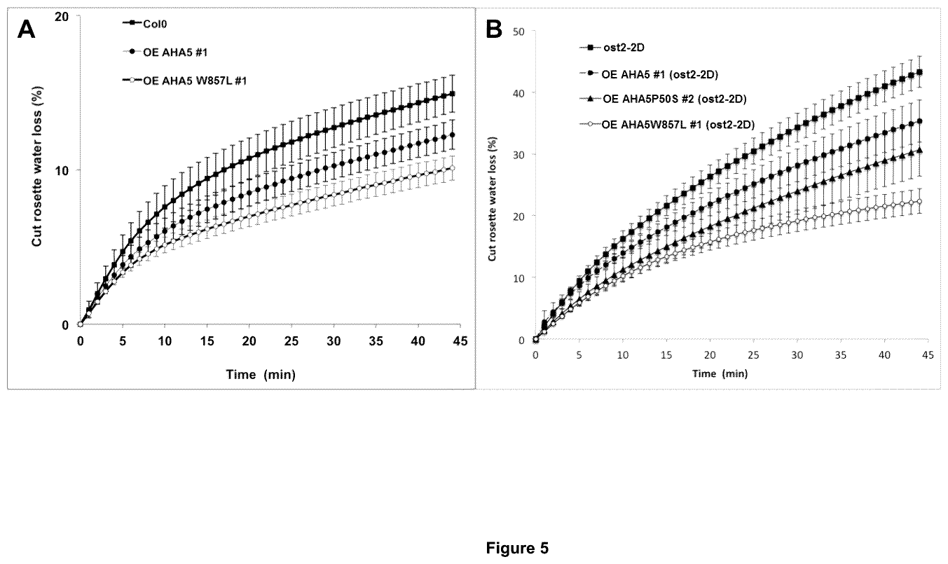

Transpirational water loss is one of the most important factors related to drought tolerance. To assess the rate of water loss, rosettes from plants (Col0 or ost2-2D) transformed with AtAHA5, AtAHA5P50S or AtAHA5W857L under the control of the 35S promoter, were detached and their fresh weight changes were measured over a 45 min period. The increase activity of AtAHA5 results in decreased water loss from excised rosettes. Rosettes from different ecotypes were grown on soil in growth chamber (21° C., 70% relative humidity, 8 h/16 h light/dark, 300 μmol.m-2.s-1) were excised at least 4 h after the beginning of the light period and transferred to darkness. Water loss was determined by monitoring the decrease in fresh weight of the excised rosettes. Means±SE of at least 3 independent measurements are shown on FIG. 5.

These data show that the leaves from plants that over-expressed AtAHA5 showed a slower rate of water loss compare to wild-type (FIG. 5A) or ost2-2D leaves (FIG. 5B). Moreover, the amino acid substitutions P50S or W857L enhance this effect showing that a constitutive activity of the AtAHA5 proton pump allows reducing rate of water loss.

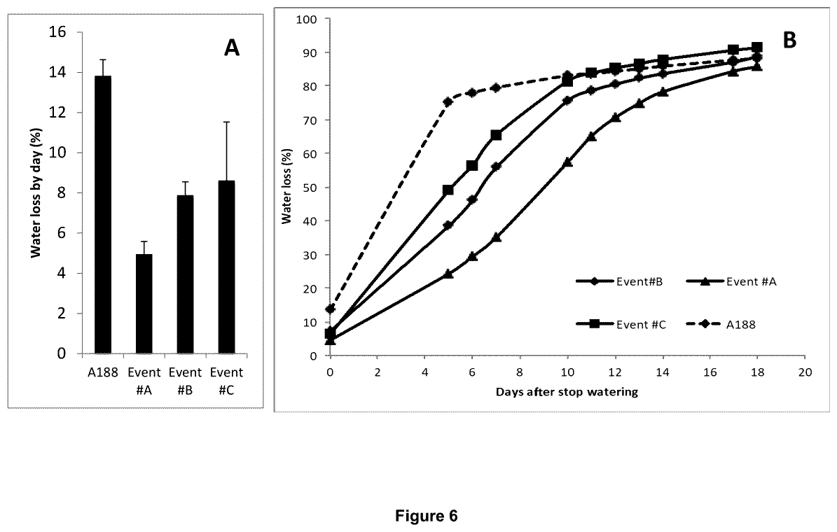

Maize plantlets transformed with ZmAHA5 W885L under the control of the promoter pZmRAB17 are grown on soil in a growth chamber under controlled conditions. After 5 weeks, plants are submitted to water stress and water loss is determined daily by monitoring the decrease of plantlets fresh weight while irrigation is stopped. The results are shown in FIG. 6A and 6B. The data show that the leaves from plantlets that overexpress ZmAHA5 W885L under the control of the promoter pZmRAB17 showed a slower rate of water loss as compared to A188 non-transformed plantlets leaves. The three events (Event #A, Event #B, Event #C) present a similar pattern of slower water loss compared to the control.

### Example 9

Comparison of the Stomatal Aperture in Normal Conditions or with ABA

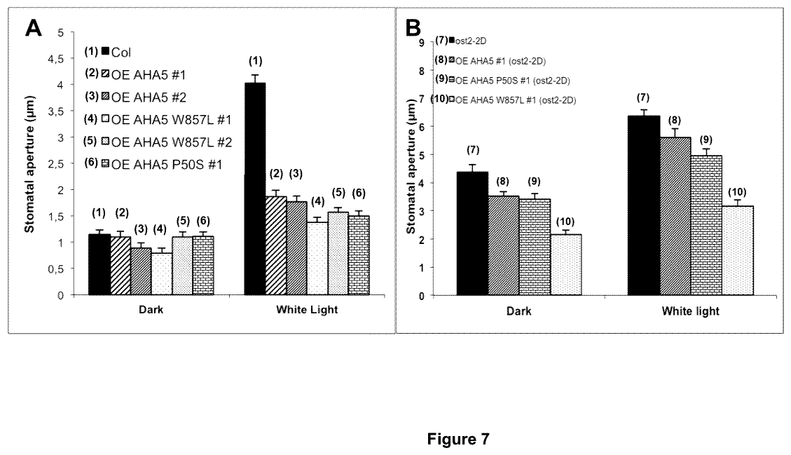

The opening and closing of stomata are controlled by environmental factors, including light, humidity and CO2 concentration, as well as by phytohormones, such as ABA (abscisic acid). Because the AtAHA5 over-expression lines exhibit a slower rate of water loss, the stomatal movement of the transformed plants was tested to compare with the controls (Col0 or ost2-2D). Stomatal aperture measurements were done on epidermis from leaves of 3-4 week-old-plants grown on soil in growth chamber (21° C., 70% relative humidity, 8 h/16 h light/dark, 300 μmol.m-2.s-1). After 30 min in darkness in 30 mM KCl, 10 mM MES-Tris pH6.0, stomatal apertures were measured. In light-induced stomatal opening experiments, epidermal peels were incubated 3 h under light (300 μmol.m-2.s-1) at 22° C. and then stomatal apertures were measured. Each value presented is the mean of at least 60 apertures from at least three independant experiments. Error bars represent standard errors to the mean (SEM) with a confidence interval of 95%. Data represent means±sem of 3 independent repetitions. Results are shown on FIG. 7.

Thus, the stomatal aperture of the transformed plants with several versions of AtAHA5 under the control of the 35S promoter was measured in different conditions and compared to wild-type (FIG. 7A) or ost2-2D (FIG. 7B).

In the dark, the apertures of all plants were similar compare to wild type plants but strongly affected in ost2-2D mutants suggesting that the AtAHA5 proton pump activity abolish the ost2-2D phenotype. After a 3 hours illumination, the stomatal aperture in the AtAHA5 over-expression lines was found to be greatly reduced compared to wild-type (FIG. 7A) or ost2-2D plants (FIG. 7B). Altogether, these results demonstrate that AtAHA5 activity inhibits light-induce stomatal opening. The size of the guard cells and the density of stomata were determined and both parameters are similar between transgenic lines and wild-type plants (data not shown).

### Example 10

### Biomass Experiments

Plant stomata control both carbon uptake and transpiration via opening and closure. Therefore when stomatal pore aperture is reduced due to stomatal closure, gas exchanges may be affected putatively limiting carbon uptake. Such reduction of gas exchanges may be translated in a lower photosynthesis rate and plant biomass. In order to evaluate the impact of the ectopic expression of AtAHA5 or its constitutively active variant on plant biomass, Arabidopsis plants (Col0 or ost2-2D) transformed with AtAHA5, AtAHA5P50S or AtAHA5W857L under the control of the 35S promoter, were grown on soil in growth chamber (21° C., 70% relative humidity, 8 h/16 h light/dark, 300 μmol.m-2.s-1) during 4 weeks and rosettes were excised at least 4 h after the beginning of the light period and transferred to darkness. The fresh weight of the excised rosettes was measured. Means and standard deviation are given (n≥6 plant repeats).

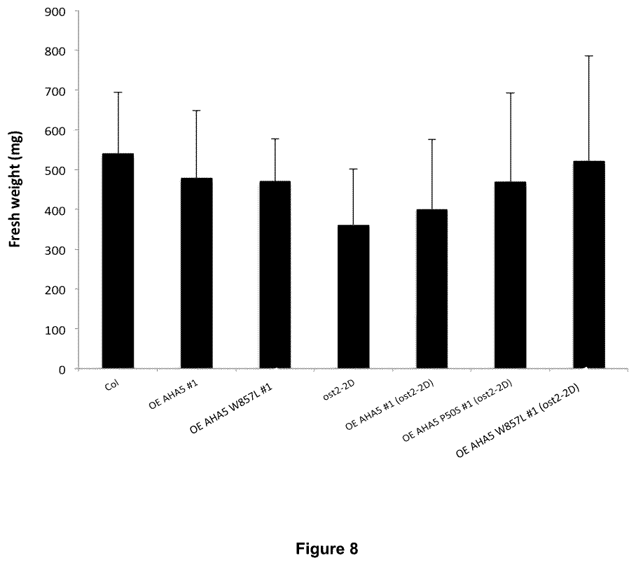

The increase activity of AtAHA5 or a constitutive variant AtAHA5 does not affect plant biomass in standard conditions. The biomass of the plants over-expressing AtAHA5 or its constitutive variant is not affected (FIG. 8)

### Example 11

### Characterization of the Promoter SbMYB60

Segregating T1 or T2 seeds expressing the pSbMYB60: ZsGreen cassette and nulls are sowed in pots with fertilizer and placed in a greenhouse controlled environment.

Selection of the transgenic plants was carried out through herbicide resistance test.

12 transgenic plants per events are grown under normal irrigation of 5 minutes twice a day until the emergence of the 15th leaf. Then two lots of 6 plants per event undergo two different irrigation treatments. A lot is irrigated as explained above while the irrigation of the second lot is reduced to 1 minute 30 twice a day until panicle emergence then stopped until flowering stage. The leaf sampling is performed once plants are severely stressed at flowering stage.

In order to evaluate ZsGreen (Clonetech; Matz et al., 1999) expression in response to water deficit on younger leaf, drought stress experiments were also carried out on maize seedlings. Leaf 4 of 7 events were sampled concomitantly.

### A—RT-PCR Analysis

Total RNAs are purified using SV 96 Total RNA Isolation System (Promega). A DNase treatment is applied and the integrity of the RNA is controlled. The cDNA are synthesized by reverse transcription using MultiScribe™ Reverse Transcriptase (Thermo Fisher Scientific).

ZsGreen transcripts are quantified by quantitative RT-PCR (cycle of 10 minutes at 25° C., 2 hours at 37° C., 5 minutes at 85° C.). Relative expression was normalized to ZmUbiquitin (GRMZM2G110983) and ZmEF1α (GRMZM2G001327) and calculated according to ΔΔCt method (Livak et al, 2001).

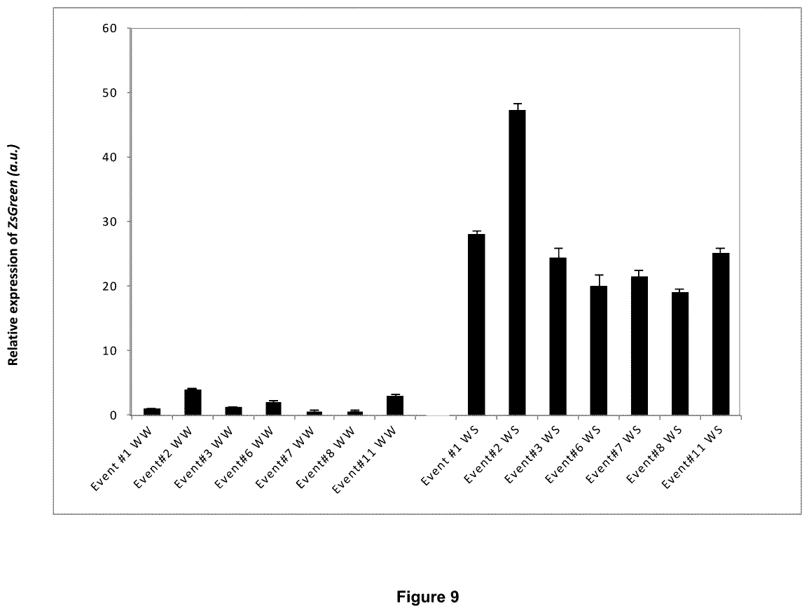

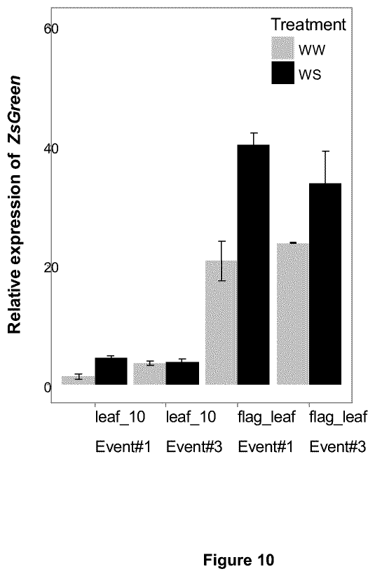

As seen in FIG. 9, expression of ZsGreen is significantly up-regulated at about 30 fold under stress conditions in young leaf (leaf #4) and reaching 50 fold for event 2. As seen in FIG. 10, expression of ZsGreen is found up-regulated at about 1.5 to 2 fold in older leaf (leaf #10 and flag leaf). Results are significant (p-value>0.01) except for leaf #10 of event 003. The level of ZsGreen cDNA in the tenth leaf of the stressed plants is statistically higher than in irrigated ones. This observation is identical for the flag leaf.

### B—Confocal Microscopy Observation

Plants are grown as described above and confocal microscopy observation is carried out once plants are severely stressed. Adaxial transgenic plant leaf epidermis are isolated on a microscope slide. Slide are mounted in a mounting buffer (glycerol 50%, PBS 0,5×). The stomata are observed by confocal microscopy using a LSM 800 Airyscan (Carl Zeiss), excitation wavelength was 488 nm, a pinhole of 38 μm at 1 airy unit, emission was detected between 494 and 520 nm.

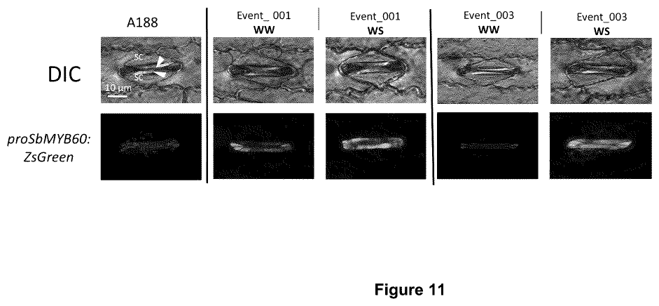

As seen in FIG. 11, the fluorescence signal detected from observation of stressed plants guard cells is greater than that of well irrigated plants guard cells showing that the ZsGreen was more up-regulated in stressed plants.

These results indicate that the fragment of pSbMYB60 promoter used for transformation not only drives a guard-cell specific expression but yet drives an up-regulation of the downstream gene in response to water deficit. This promoter can be considered as a new drought inducible promoter.

### REFERENCES

### Patent literature

EP2995680
WO2014/012145
EP1033405
US 20130074202
US 20090217414
U.S. Pat. No. 8,577,624

### Non-patent literature

Alsterfjord et al. Plant Cell Physiol (2004) 45 (9): 1202-1210
Axelsen et al. Plant Physiology 2001 June;126 (2): 696-706
Baunsgaard et al. The Plant Journal Volume 10, Issue 3 Sep. 1996 Pages 451-458

Baxter et al. PNAS (2005) vol. 102 no. 7 2649-2654
Caldeira et al. Plant Physiology 2014 April; 164 (4):1718-30
Cid et al. Current Genetics June 1987, Volume 12, Issue 2, pp 105-110
Clément M et al. Plant Physiology 2011 July; 156 (3):1481-92
DeWitt et al. The Plant Cell, Vol. 7, 2053-2067 December 1995
Gouy M., Guindon S. & Gascuel O. (2010) Molecular Biology and Evolution 27 (2):221-224
Guindon S., Dufayard J. F., Lefort V., Anisimova M., Hordijk W., Gascuel O. 2010, Systematic Biology, 59 (3):307-21
Haruta et al. J Biol Chem. 2010 Jun. 4; 285 (23): 17918-17929.
Haruta et al. Curr Opin Plant Biol. 2015 December; 28:68-75
Houlne et al. The Plant Journal Volume 5, Issue 3 Mar. 1994 Pages 311-317
Ishida et al. Nature Biotechnology, 14, 745-750, 1996
Jenkins et al. Plant, Cell and Environment (1999) 22, 159-167
Komari et al. (1996) The Plant Journal (1996) 10 (1), 165-174
Leonhardt N et al. Proc Natl Acad Sci U S A. 1997 Dec. 9;94 (25):14156-61
Leonhardt et al, The Plant Cell, Vol. 11, 1141-1151 June 1999
Livak et al, Methods 25,402-408 (2001)
Matsuoka and Sanada, 1991 Mol Gen Genet. 225:411-9
Matz et al. 1999 Nature Biotechnology 17, 969-973
McElroy et al 1990 The Plant Cell, Vol. 2, 163-171, February 1990
Merlot S et al. (2002) Plant Journal 30:601-609
Merlot et al., 2007 The EMBO Journal Volume 26, Issue 13, 11 Jul. 2007, pp 3,039-3271

Perfus-Barbeoch L et al. Plant J. 2002 November; 32 (4):539-48
Rusconi et al.2013 J. Exp. Bot. 64:3361-71
Ueno et al. Plant Cell Physiol (2005) 46 (6): 955-963
Verdaguer et al (1996) Plant Molecular Biology September 1996, Volume 31, Issue 6, pp 1129-1139
Vilardell et al. Plant Mol Biol. 17:985-93
Vitart et al. The Plant Journal (2001) 27(3), 191-201
Weigel and Glazebrook. In Planta Transformation of Arabidopsis. 2006 Cold Spring Harbor Protocols

1. A nucleic acid construct comprising a transgene operably linked to a nucleic acid sequence encoding a promoter as set forth in SEQ ID NO:41 or with at least 80% identity with SEQ ID NO: 40, which is functional in plants and allows expression in guard cells.

2. The nucleic acid construct of claim 1, wherein the promoter is as set forth in SEQ ID NO:41 or has at least 90% identity with SEQ ID NO: 40.

3. The nucleic acid construct of claim 1, wherein the promoter is as set forth in SEQ ID NO:41 or has at least 95% identity with SEQ ID NO: 40.

4. The nucleic acid construct of claim 1, wherein the promoter is not the endogenous promoter of the transgene.

5. The nucleic acid construct of claim 2, wherein the promoter is not the endogenous promoter of the transgene.

6. The nucleic acid construct of claim 3, wherein the promoter is not the endogenous promoter of the transgene.

7. The nucleic acid construct of claim 1, wherein the promoter is a guard cell specific promoter.

8. The nucleic acid construct of claim 1, wherein the promoter is a drought inducible promoter.

9. The nucleic acid construct of claim 1, wherein the promoter comprises the nucleic acid sequence of SEQ ID NO:41.

10. A transgenic plant comprising the nucleic acid construct of claim 1.

11. A transgenic plant comprising the nucleic acid construct of claim 2.

12. A transgenic plant comprising the nucleic acid construct of claim 3.

13. A transgenic plant comprising the nucleic acid construct of claim 4.

14. A transgenic plant comprising the nucleic acid construct of claim 5.

15. A transgenic plant comprising the nucleic acid construct of claim 6.

16. A method for expressing a transgene in guard cells of a plant comprising introducing the nucleic acid construct of claim 4 into the plant.

17. A method for expressing a transgene in guard cells of a plant comprising introducing the nucleic acid construct of claim 5 into the plant.

18. A method for expressing a transgene in guard cells of a plant comprising introducing the nucleic acid construct of claim 6 into the plant.

19. The method of claim 16, further comprising detecting expression of the transgene.

20. The method of claim 17, further comprising detecting expression of the transgene.

21. The method of claim 18, further comprising detecting expression of the transgene.

22. The method of claim 19, comprising transforming a plant cell with the nucleic acid construct, and regenerating said cell to obtain a whole plant.

23. The method of claim 20, comprising transforming a plant cell with the nucleic acid construct, and regenerating said cell to obtain a whole plant.


SEQUENCE LISTING (66 sequences)

SEQ ID NO: 1 - protein, 931 aa, Arabidopsis thaliana
  REGION 1..931: note = misc_feature - AtAHA5 wild-type
MEEVFEELKCTKQGLTANEASHRLDVFGPNKLEEKKESKLLKFLGFMWNPLSWVMEVAALMAIALANGGGRPPDWQDFVGIVCLLLINSTISFIEENNAGNAAAALMAGLAPKTKVLRDNQWSEQEASILVPGDVISIKLGDIIPADARLLDGDPLKIDQSSLTGESIPVTKNPSDEVFSGSICKQGEIEAIVIATGVHTFFGKAAHLVDNTNQIGHFQKVLTSIGNFCICSIALGIIVELLVMYPIQRRRYRDGIDNLLVLLIGGIPIAMPSVLSVTMATGSHRLFQQGAITKRMTAIEEMAGMDVLCCDKTGTLTLNKLTVDKNLVEVFAKGVGKEHVFLLAARASRIENQDAIDAAIVGMLADPKEARAGVREVHFFPFNPVDKRTALTYVDSDGNWHRASKGAPEQILNLCNCKEDVRRKVHGVIDKFAERGLRSLAVARQEVLEKKKDAPGGPWQLVGLLPLFDPPRHDSAETIRRALNLGVNVKMITGDQLAIGKETGRRLGMGTNMYPSSALLGQVKDSSLGALPVDELIEKADGFAGVFPEHKYEIVHRLQQRNHICGMTGDGVNDAPALKKADIGIAVVDATDAARGASDIVLTEPGLSVIISAVLTSRAIFQRMKNYTIYAVSITIRIVFGFMFIALIWQFDFSPFMVLIIAILNDGTIMTISKDRMKPSPQPDSWKLRDIFSTGVVLGGYQALMTVVFFWVMKDSDFFSNYFGVRPLSQRPEQMMAALYLQVSIISQALIFVTRSRSWSYAECPGLLLLGAFVIAQLVATFIAVYANWSFARIEGAGWGWAGVIWLYSFLTYIPLDLLKFGIRYVLSGKAWLNLLENKTAFTTKKDYGKEEREAQWAAAQRTLHGLQPAEKNNIFNEKNSYSELSQIAEQAKRRAEVVRLREINTLKGHVESVVKLKGLDIDTIQQHYTV

SEQ ID NO: 2 - protein, 961 aa, Zea mays
  REGION 1..961: note = misc_feature - ZmAHA5 wild-type
MGPLQRRPTAMGGLEEIKNEAVDLENIPIEEVFEQLKCTREGLSSSEGQQRLEIFGPNRLEEKKESKILKFLGFMWNPLSWVMEMAAVMAIALANGGGKPPDWEDFVGIIVLLVINSTISFIEENNAGNAAAALMANLAPKTKVLRDGRWGEQEAAILVPGDIVSIKLGDIVPADARLLEGDPLKVDQSALTGESLPVTKGPGDEVFSGSTCKQGEIEAVVIATGVHTFFGKAAHLVDSTNQVGHFQQVLTAIGNFCICSIAVGIVVEIIVMFPIQHRRYRSGIENLLVLLIGGIPIAMPTVLSVTMAIGSHKLSQQGAITKRMTAIEEMAGMDVLCSDKTGTLTLNKLSVDKNLVEVFCKGVDKDHVLLLAARASRTENQDAIDAAMVGMLADPKEARAGIREIHFLPFNPVDKRTALTYIDADGHWHRVSKGAPEQILDLCHCKEDLRRKVHGIIDKYAERGLRSLAVARQEVPERNKESPGGPWQFVGLLPLFDPPRHDSAETIRKALVLGVNVKMITGDQLAIGKETGRRLGMGTNMYPSSALLGQNKDATLEALPVDELIEKADGFAGVFPEHKYEIVKRLQEKKHIVGMTGDGVNDAPALKKADIGIAVADATDAARSASDIVLTEPGLSVIISAVLTSRCIFQRMKNYTIYAVSITIRIVLGFMLIALIWQYDFSPFMVLIIAILNDGTIMTISKDRVKPSPLPDSWKLKEIFATGIVLGSYLALMTVIFFWAMHKTDFFSDKFGVRSIRDSEHEMMSALYLQVSIVSQALIFVTRSRSWSFVERPGLLLVTAFLLAQLVATFLAVYANWGFARIKGIGWGWAGVVWLYSIVFYFPLDLIKFFIRFVLSGRAWDNLLENKTAFTTKKDYGREEREAQWATAQRTLHGLQPPEAATSTLFHDKNSYRELSEIAEQAKRRAEIARLRELNTLKGHVESVVKLKGLDIDTIQQNYTV

SEQ ID NO: 3 - protein, 954 aa, Helianthus annuus
  REGION 1..954: note = misc_feature - HaRA11741_4232_Helianthus_annuus
MGTDKALGLEEIKNETVDLEKVPIEEVFQQLKCNREGLTSDEGAARLQIFGPNKLEEKKESKILKFLGFMWNPLSWVMEAAAIMAIAMANGGGKPPDWQDFVGIVCLLVINSTISFIEENNAGNAAAALMAGLAPKTKVLRDGRWSEQEASILVPGDIISIKLGDIVPADARLLEGDPLKIDQSALTGESLPVNKNPYDEVFSGSTCKQGEIEAVVIATGVHTFFGKAAHLVDSTNQVGHFQQVLTAIGNFCICSIAVGMLVEIIVMYPIQHRQYRSGIDNLLVLLIGGIPIAMPTVLSVTMAIGSHRLSQQGAITKRMTAIEEMAGMDVLCSDKTGTLTLNKLTVDKNLIEVFAKGVDKEQVLLYAARASRTENQDAIDAAIVGTLADPKEARAGIREVHFFPFNPVDKRTALTYIDERGNWHRTSKGAPEQILTLCGCKEDMKKKVHAMIDKFAERGLRSLAVGKQEVPEKTKEGPGGPWQFVGLLSLFDPPRHDSAETIRQALHLGVNVKMITGDQLAIAKETGRRLGMGTNMYPSSSLLGDHKDASIATLPIEELIEKADGFAGVFPEHKYEIVKKLQERKHICGMTGDGVNDAPALKKADIGIAVADATDAARSASDIVLTEPGLSVIISAVLTSRAIFQRMKNYTIYAVSITIRIVFGFMFIALIWKFDFSPFMVLIIAILNDGTIMTISKDRVKPSPLPDSWKLKEIFATGIVLGGYLALMTVIFFWIMKDTDFFTEKFGVRSLRNSEVEMMAALYLQVSIVSQALIFVTRSRSWSFIERPGLLLLGAFLAAQLVATLIAVYAEWEFARIKGVGWGWAGVIWLYSIVFYFPLDIMKFAIRYILSGKAWRNMLENKTAFTTKKDYGREEREAQWALAQRTLHGLQAPETSNIFNEKSSYRELSEIAEQAKRRAEVARLREVLTLKGHVESVVKLKGLDIDTIQQHYTV

SEQ ID NO: 4 - protein, 952 aa, Helianthus annuus
  REGION 1..952: note = misc_feature - HaRA11742_4232_Helianthus_annuus
MGGDKALSLEEIKNETVDLEKVPIEEVFEQLKCNREGLSSDEGAARLQIFGPNKLEEKKESKLLKFLGFMNPLSWVMEAAAIMAIALANGGGKPPDWQDFVGIVCLLVINSTISFIEENNAGNAAAALMAGLAPKTKVLRDGNWSEQEAAILVPGDIISIKLGDIVPADARLLEGDPLKIDQSALTGESLPVNKNPYDEVFSGSTCKQGIEAVVIATGVHTFFGKAAHLVDSTNQVGHFQQVLTAIGNFCICSIAVGMLVEIVVMYPIQHREYRNGIDNLLVLLIGGIPIAMPTVLSVTMAIGSHRLSQQGAITKRMTAIEEMAGMDVLCSDKTGTLTLNKLSVDKNLIEVFAKGVDKDQVLLYAARASRTENQDAIDAAIVGTLADPKEARAGIREVHFFPFNPVDKRTALTYIDERGNWHRTSKGAPEQILTLCGCKEDMKKKVHAMIDKFAERGLRSLAVAKQEVPEKNKESPGGPWTFIGLLSLFDPPRHDSAETIRRALNLGVNVKMITGDQLAIAKETGRRLGMGTNMYPSSSLLGNHKDASIAAIPIEELIEKADGFAGVFPEHKYEIVKKLQERKHICGMTGDGVNDAPALKKADIGIAVADATDAARGASDIVLTEPGLSVIISAVLTSRAIFQRMKNYTIYAVSITIRIVFGFLFIALIWKFDFSPFMVLIIAILNDGTIMTISKDRVKPSPLPDSWKLKEIFATGIVLGGYLALMTVIFFWIMKDTNFFSDTFGVRSLRHSEVEMMAALYLQVSIVSQALIFVTRSRSWSFIERPGLLLLGAFLAAQLVATLIAVYANWEFARIKGVGWGWAGVIWLYSIVFYFPLDLMKFAIRYILSGKAWNNLLENKTAFTSKKDYGREEREAQWALAQRTLHGLQQPETSNIFNEKSSYRELSEIAEQAKRRAEVARLREVLTLKGHVESVVKLKGLDIDTIQQHYTV

SEQ ID NO: 5 - protein, 952 aa, Lycopersicon esculentum
  REGION 1..952: note = misc_feature - LeAHA_XP_004242908.1_Lycopersicon_esculentum
MAKAISLEEIKNETVDLEKIPIEEVFEQLKCSREGLTSDEGANRLQIFGPNKLEEKKESKILKFLGFMWNPLSWVMEMAAIMAIALANGDGKPPDWQDFVGIVCLLVINSTISFIEENNAGNAAAALMAGLAPKTKVLRDGRWSEQEAAILVPGDIISVKLGDIVPADARLLEGDPLKIDQSALTGESLPVTKNPGDEVFSGSTCKQGELEAVVIATGVHTFFGKAAHLVDSTNNVGHFQKVLTAIGNFCICSIAIGMLVEIIVMYPIQHRKYRDGIDNLLVLLIGGIPIAMPTVLSVTMAIGSHRLSQQGAITKRMTAIEEMAGMDVLCSDKTGTLTLNKLSVDRSLVEVFTKGVDKEYVLLLAARASRVENQDAIDACMVGMLADPKEARAGIREVHFLPFNPVDKRTALTYIDSNGNWHRASKGAPEQILDLCNCKEDVRRKVHSMIDKYAERGLRSLAVARQEVPEKSKESTGGPWQFVGLLPLFDPPRHDSAETIRRALNLGVNVKMITGDQLAIAKETGRRLGMGTNMYPSASLLGQDKDSSIASLPVEELIEKADGFAGVFPEHKYEIVKKLQERKHIVGMTGDGVNDAPALKKADIGIAVADATDAARGASDIVLTEPGLSVIISAVLTSRAIFQRMKNYTIYAVSITIRIVFGFMLIALIWKYDFSAFMVLIIAILNDGTIMTISKDRVKPSPMPDSWKLNEIFATGVVLGGYQALMTVIFFWAMHDTSFFTDKFGVKDIRESDEEMMSALYLQVSIISQALIFVTRSRSWSFVERPGALLMIAFLIAQLVATLIAVYADWTFARVKGCGWGWAGVIWIFSIVTYFPLDIMKFAIRYILSGKAWNNLLDNKTAFTTKKDYGKEEREAQWALAQRTLHGLQPPEASNLFNEKNSYRELSEIAEQAKRRAEMARLRELHTLKGHVESVVKLKGLDIETIQQHYTV

SEQ ID NO: 6 - protein, 951 aa, Aeluropus littoralis
  REGION 1..951: note = misc_feature - AlAHA_G3M3G8_Aeluropus_littoralis
MGGLEEIKNEAVDLENIPMEEVFEQLKCTREGLSSEEGTQRLQVFGPNKLEEKKESKVLKFLGFMWNPLSWVMEMAAIMAIALANGGGKPPDWQDFVGIIVLLVINSTISFIEENNAGNAAAALMANLAPKTKVLRDGRWGEQEAAILVPGDIISIKLGDIVPADARLLEGDPLKVDQSALTGESLPVTKGPGDEVFSGSTCKQGEIEAVVIATGVHTFFGKAAHLVDSTNQVGHFQQVLTAIGNFCICSIAVGIVIEIIVMFPIQHRKYRSGIENLLVLLIGGIPIAMPTVLSVTMAIGSHKLSQQGAITKRMTAIEEMAGMDVLCSDKTGTLTLNKLSVDKNLVEVFAKGVDKDHVLLLAARASRTENQDAIDAAMVGMLADPKEARAGIREVHFLPFNPVDKRTALTYIDSDGNWHRASKGAPEQIITLCNCREDMKRKVHSIIDKYAERGLRSLAVARQEVPEKTKESPGGPWQFVGLLPLFDPPRHDSAETIRKALHLGVNVKMITGDQLAIGKETGRRLGMGTNMYPSSALLGQNKDSTLEALPVDELIEKADGFAGVFPEHKYEIVKKLQEKKHIVGMTGDGVNDAPALKKADIGIAVADATDAARSASDIVLTEPGLSVIISAVLTSRCIFQRMKNYTIYAVSITIRIVLGFLLIALIWKFDFAPFMVLIIAILNDGTIMTISKDRVKPSPLPDSWKLNEIFATGIVLGTYLAIMTVVFFWAIHKTDFFTEKFGVRSIRDSEHEMMAALYLQVSIVSQALIFVTRSRSWSFVERPGVLLVTAFLLAQLVATLIAVYANWGFARIKGIGWGWAGVVWLYSVVLYFPLDVFKFLIRFALSGRAWDNLLENKTAFTTKKDYGREEREAQWAAAQRTLHGLQPPEVASNTLFNEKSSYRELSEIAEQAKRRAEIARLRELNTLKGHVESVVKLKGLDIDTIQQNYTV

SEQ ID NO: 7 - protein, 951 aa, Aeluropus littoralis
  REGION 1..951: note = misc_feature - AlAHA_G9MC80_Aeluropus_littoralis
MGGLEEIKNESVDLENIPMEEVFEQLKCTREGLSSEEGTQRLQVFGPNKLEEKKESKVLKFLGFMWNPLSWVMEMAAIMAIALANGGGKPPDWQDFVGIIVLLVINSTISFIEENNAGNAAAALMANLAPKTKVLRDGRWGEQEAAILVPGDIISIKLGDIVPADARLLEGDPLKVDQSALTGESLPVTKGPGDEVFSGSTCKQGEIEAVVIATGVHTFFGKAAHLVDSTNQVGLFQQVLTAIGNFCICSIAVGIVIEIIVMFPIQHRRYRSGIENLLVLLIGGIPIAMPTVLSVTMAIGSHKLSQQGAITKRMTAIEEMAGMDVLCSDKTGTLTLNKLSVDKNLVEVFAKGVDKDHVLLLAARASRTENQDAIDAAMVGMLADPKEARAGIREVHFLPFNPVDKRTALTYIDSDGNWHRASKGAPEQIITLCNCREDMKRKVHSIIDKYAERGLRSLAVARQEVPEKTKESPGGPWQFVGLLPLFDPPRHDSAETIRKALHLGVNVEMITGDQLAIGKETGRRLGMGTNMYPSSALLGQNKDATLEALPVDELIEKADGFAGVFPEHKYEIVKKLQEKKHIVGMTGDGVNDAPALKKADIGIAVADATDAARGASDIVLTEPGLSVIISAVLTSRCIFQRMKNYTIYAVSITIRIVLGFLLIALIWKFDLAPFMVLIIAILNDGTIMTISKDRVKPSPLPDSWKLKEIFATGIVLGTYLAIMTVVFFWAIHKTDFFTEKFGVRSIRDSEDEMMAALYLQVSIVSQALIFVTRSRSWSFVERPGVLLVTAFLLAQLVATLIAVYADWGFARIKGIGWGWAGVVWLYSVVFYLPLDVFKFLIRFALSGRAWDNLLENKTAFTTKKDYGREEREAQWAAAQRTLHGLQPPEVASNTLFNEKSSYRELSEIAEQAKRRAEIARLRELNTLKGHVESVVKLKGLDIDTIQQNYTV

SEQ ID NO: 8 - protein, 951 aa, Sorghum bicolor
  REGION 1..951: note = misc_feature - SbAHA_Sb06g031240_Sorghum_bicolor
MGGLEEIKNEAVDLENIPIEEVFEQLKCTREGLSSSEGQQRLEIFGPNKLEEKKESKILKFLGFMWNPLSWVMEMAAIMAIALANGGGKPPDWEDFVGIIVLLVINSTISFIEENNAGNAAAALMANLAPKTKVLRDGRWGEQEAAILVPGDIISIKLGDIVPADARLLEGDPLKVDQSALTGESLPVTRGPGDEVFSGSTCKQGEIEAVVIATGVHTFFGKAAHLVDSTNQVGHFQQVLTAIGNFCICSIAVGIIVEIIVMFPIQHRKYRSGIENLLVLLIGGIPIAMPTVLSVTMAIGSHKLSQQGAITKRMTAIEEMAGMDVLCSDKTGTLTLNKLSVDKNLVEVFCKGVDKDHVLLLAARASRTENQDAIDAAMVGMLADPKEARAGIREVHFLPFNPVDKRTALTYIDGDGNWHRVSKGAPEQILDLCHCKEDLRRKVHGIIDKYAERGLRSLAVARQEVPEKNKESPGGPWQFVGLLPLFDPPRHDSAETIRKALVLGVNVKMITGDQLAIGKETGRRLGMGTNMYPSSALLGQNKDSTLEALPVDELIEKADGFAGVFPEHKYEIVKRLQEKKHIVGMTGDGVNDAPALKKADIGIAVADATDAARSASDIVLTEPGLSVIISAVLTSRCIFQRMKNYTIYAVSITIRIVLGFMLIALIWKYDFSPFMVLIIAILNDGTIMTISKDRVKPSPLPDSWKLKEIFATGIVLGSYLAIMTVIFFWAMHKTDFFSDKFGVRSIRDSEHEMMSALYLQVSIVSQALIFVTRSRSWSFVERPGLLLVTAFLLAQLVATFLAVYANWGFARIKGIGWGWAGVVWLYSIVFYFPLDLIKFFIRFVLSGRAWDNLLENKTAFTTKKDYGREEREAQWATAQRTLHGLQPPEAATNTLFNDKSSYRELSEIAEQAKRRAEIARLRELNTLKGHVESVVKLKGLDIDTIQQNYTV

SEQ ID NO: 9 - protein, 951 aa, Setaria italica
  REGION 1..951: note = misc_feature - SiAHA_K3Z3J5_Setaria_italica
MGGLEEIKNEAVDLENIPIEEVFEQLKCTREGLSSNEGAQRLEIFGPNKLEEKKESKILKFLGFMWNPLSWVMEMAAIMAIALANGGGKPPDWEDFVGIIVLLVINSTISFIEENNAGNAAAALMANLAPKTKVLRDGRWGEQEAAILVPGDIISIKLGDIVPADARLLEGDPLKVDQSALTGESLPVTKGPGDEVFSGSTCKQGEIEAVVIATGVHTFFGKAAHLVDSTNQVGHFQKVLTAIGNFCICSIAVGIVIEIIVMFPIQHRAYRSGIENLLVLLIGGIPIAMPTVLSVTMAIGSHKLSQQGAITKRMTAIEEMAGMDVLCSDKTGTLTLNKLSVDKNLVEVFCKGVDKDHVLLLAARASRTENQDAIDAAMVGMLADPKEARAGIREVHFLPFNPVDKRTALTYIDSDGNWHRVSKGAPEQILDLCNCREDMRRKVHSIIDKYAERGLRSLAVARQEVPEKSKDASGGPWQFVGLLPLFDPPRHDSAETIRKALVLGVNVKMITGDQLAIGKETGRRLGMGTNMYPSSSLLGTNKDSTLESLPVDELIEKADGFAGVFPEHKYEIVKRLQEKKHIVGMTGDGVNDAPALKKADIGIAVADATDAARSASDIVLTEPGLSVIISAVLTSRCIFQRMKNYTIYAVSITIRIVLGFMLIALIWKYDFSPFMVLIIAILNDGTIMTISKDRVKPSPLPDSWKLKEIFATGVVLGSYLALMTVIFFWAMHKTDFFPEKFGVKPIRDSEGKMMSALYLQVSIVSQALIFVTRSRSWSFVERPGLLLVTAFLLAQLVATFLAVYANWGFARIEGIGWGWAGVVWLYSIVFYFPLDLFKFFIRFVLSGRAWDNLLENKTAFTTKKDYGREEREAQWATAQRTLHGLQPPEAASNTLFNDKSSYRELSEIAEQAKRRAEIARLRELNTLKGHVESVVKLKGLDIDTIQQNYTV

SEQ ID NO: 10 - protein, 951 aa, Oryza sativa
  REGION 1..951: note = misc_feature - Os04g56160_Oryza_sativa
MGGLEEIKNEAVDLENIPIEEVFEQLKCTREGLSSEEGNRRIEMFGPNKLEEKKESKILKFLGFMWNPLSWVMEMAAIMAIALANGGGKPPDWEDFVGIIVLLVINSTISFIEENNAGNAAAALMANLAPKTKVLRDGRWGEQEAAILVPGDIISIKLGDIVPADARLLEGDPLKIDQSALTGESLPVTKNPGDEVFSGSTCKQGEIEAVVIATGVHTFFGKAAHLVDSTNQVGHFQTVLTAIGNFCICSIAVGIVIEIIVMFPIQHRAYRSGIENLLVLLIGGIPIAMPTVLSVTMAIGSHKLSQQGAITKRMTAIEEMAGMDVLCSDKTGTLTLNKLSVDKNLVEVFTKGVDKDHVLLLAARASRTENQDAIDAAMVGMLADPKEARAGIREVHFLPFNPVDKRTALTYIDADGNWHRASKGAPEQILTLCNCKEDVKRKVHAVIDKYAERGLRSLAVARQEVPEKSKESAGGPWQFVGLLPLFDPPRHDSAETIRKALHLGVNVKMITGDQLAIGKETGRRLGMGTNMYPSSALLGQNKDASLEALPVDELIEKADGFAGVFPEHKYEIVKRLQEKKHIVGMTGDGVNDAPALKKADIGIAVADATDAARSASDIVLTEPGLSVIISAVLTSRCIFQRMKNYTIYAVSITIRIVLGFLLIALIWKYDFSPFMVLIIAILNDGTIMTISKDRVKPSPLPDSWKLKEIFATGIVLGSYLALMTVIFFWAMHKTDFFTDKFGVRSIRNSEHEMMSALYLQVSIVSQALIFVTRSRSWSFIERPGLLLVTAFMLAQLVATFLAVYANWGFARIKGIGWGWAGVIWLYSIVFYFPLDIFKFFIRFVLSGRAWDNLLENKIAFTTKKDYGREEREAQWATAQRTLHGLQPPEVASNTLFNDKSSYRELSEIAEQAKRRAEIARLRELNTLKGHVESVVKLKGLDIDTIQQNYTV

SEQ ID NO: 11 - protein, 951 aa, Brachypodium distachyon
  REGION 1..951: note = misc_feature - BdAHA_Bradi5g24690.1_Brachypodium_distachyon
MGGLEEIRNEAVDLENIPIEEVFEQLKCTREGLTSDEGAQRVTIFGLNKLEEKKESKVLKFLGFMWNPLSWVMEMAAIMAIALANGEGKPPDWQDFVGIIVLLVINSTISFIEENNAGNAAAALMANLAPKTKVLRDGRWGEQEASILVPGDIVSIKLGDIVPADARLLEGDPLKIDQSGLTGESLPVTKNPGDEVFSGSTCKQGEIEAVVIATGVHTFFGKAAHLVDSTNQVGHFQKVLTAIGNFCIVSIAVGIVIEIIVMFPIQRRKYRAGIENLLVLLIGGIPIAMPTVLSVTMAIGSHKLSQQGAITKRMTAIEELAGMDVLCSDKTGTLTLNKLSVDKNLVEVFAKGVDKEHVLLLAARASRVENQDAIDACMVGMLADPKEARAGIREVHFLPFNPTDKRTALTYIDAEGNWHRASKGAPEQIITLCNCKEDVKRKVHSVIEKYAERGLRSLAVARQEVPEKSKDSPGGPWQFIGLLPLFDPPRHDSAETIRKALVLGVNVKMITGDQLAIGKETGRRLGMGTNMYPSSALLGQSKDGSLESLPVDELIEKADGFAGVFPEHKYEIVKRLQEKKHIVGMTGDGVNDAPALKKADIGIAVDDATDAARSASDIVLTEPGLSVIISAVLTSRCIFQRMKNYTIYAVSITIRIVLGFMLIALIWKFDFAPFMVLIIAILNDGTIMTISKDRVKPSPLPDSWKLNEIFATGVVLGTYLALMTVVFFWAIHKTDFFTNKFGVRSIRNSEFELMSALYLQVSIVSQALIFVTRSRSWSFVERPGFLLVTAFLLAQLVATLIAVYANWEFARIKGIGWGWAGVIWLFSIVFYFPLDVFKFFIRFVLSGRAWDNLLQNKTAFTTKKDYGRGEREAQWATAQRTLHGLQAPESNNNTLFNDKSSYRELSEIAEQAKRRAEIARLRELNTLKGHVESVVKLKGLDIDTINQNYTV

SEQ ID NO: 12 - protein, 951 aa, Hordeum vulgare
  REGION 1..951: note = misc_feature - HvAHA_M0Z2H5_Hordeum_vulgare
MGGLEEIRNEAVDLENIPIEEVFEQLKCTRQGLTSDEGAQRVEIFGLNKLEEKKESKVLKFLGFMWNPLSWVMEMAAIMAIALANGGGKPPDWQDFVGIIVLLVINSTISFIEENNAGNAAAALMANLAPKTKVLRDGRWGEQEASILVPGDIVSIKLGDIVPADARLLEGDPLKIDQSGLTGESLPVTKNPGDEVFSGSTCKQGEIEAVVIATGVHTFFGKAAHLVDSTNQVGHFQQVLTAIGNFCIISIAVGIVIEIIVMFPIQRRKYRAGIENLLVLLIGGIPIAMPTVLSVTMAIGSHKLSQQGAITKRMTAIEELAGMDVLCSDKTGTLTLNKLSVDKNLVEVFAKGVDKEHVLLLAARASRVENQDAIDACMVGMLADPKEARAGIREVHFLPFNPTDKRTALTYIDAEGNWHRASKGAPEQIITLCNCKEDVKRKVHSVIEKYAERGLRSLAVARQEVPEKSKDSAGGPWQFIGLLPLFDPPRHDSAETIRKALVLGVNVKMITGDQLAIGKETGRRLGMGTNMYPSSALLGQSKDGSLESLPVDELIEKADGFAGVFPEHKYEIVKRLQEKKHIVGMTGDGVNDAPALKKADIGIAVDDATDAARSASDIVLTEPGLSVIISAVLTSRCIFQRMKNYTIYAVSITIRIVLGFLLIALIWKFDFAPFMVLIIAILNDGTIMTISKDRVKPSPLPDSWKLNEIFATGVVLGTYLALMTVVFFWIIHRTDFFTNKFGVRSIRENETEKMSALYLQVSIVSQALIFVTRSRSWSFVERPGFLLVIAFLLAQLVATLIAVYANWGFARISGIGWGWAGVIWLFSIVFYFPLDIFKFFIRFVLSGRAWDNLLQNKTAFTTKENYGKGEREAQWATAQRTLHGLQAPEPASHTLFNDKSSYRELSEIAEQAKRRAEIARLRELNTLKGHVESVVKLKGLDIDTINQNYTV

SEQ ID NO: 13 - protein, 956 aa, Hordeum vulgare
  REGION 1..956: note = misc_feature - HvAHA_Q8H1X2_Hordeum_vulgare
MADKEAGNLEAVLKEVVDLENIPLEEVLDNLRCSREGLTAEQAQQRLQILGPNKLEEKEESKFLKFLGFMWNPLSWVMEMAAIMAIALANGGGKPPDWQDFVGIIVLLVINSTVSFIEENNAGNAAAALMANLAPKTKVLRDGRWGEQEASILVPGTLSASSLVTSSLLMLVCLEGDPFEDSSVWAYRRVSPSDQEPWGEVFSGSTCKQGEIEAVVIATGVHTFFGKAAHLVDSTNQVGHFQQVLTAIGNFCIISIAVGIVIEIIVMFPIQRRKYRAGIENLLVLLIGGIPIAMPTVLSVTMAIGSHKLSQQGAITKRMTAIEELAGMDVLCSDKTGTLTLNKLSVDKNLVEVFAKGVDKEHVLLLAARASRVENQDAIDACMVGMLADPKEARAGIREVHFLPFNPTDKRTALTYIDAEGNWHRASKGAPEQIITLCNCKEDVKRKVHSVIEKYAERGLRSLAVARQEVPEKSKDSAGGPWQFIGLLPLFDPPRHDSAETIRKALVLGVNVKMITGDQLAIGKETGRRLGMGTNMYPSSALLGQSKDGSLESLPVDELIEKADGFAGVFPEHKYEIVKRLQEKKHIVGMTGDGVNDAPALKKADIGIAVDDATDAARSASDIVLTEPGLSVIISAVLTSRCIFQRMKNYTIYAVSITIRIVLGFLLIALIWKFDFAPFMVLIIAILNDGTIMTISKDRVKPSPLPDSWKLNEIFATGVVLGTYLALMTVVFFWIIHRTDFFTNKFGVRSIRENETEKMSALYLQVSIVSQALIFVTRSRSWSFVERPGFLLVIAFLLAQLVATLIAVYANWGFARISGIGWGWAGVIWLFSIVFYFPLDIFKFFIRFVLSGRAWDNLLQNKTAFTTKENYGKGEREAQWATAQRTLHGLQAPEPASHTLFNDKSSYRELSEIAEQAKRRAEIARLRELNTLKGHVESVVKLKGLDIDTINQNYTV

SEQ ID NO: 14 - protein, 880 aa, Triticum aestivum
  REGION 1..880: note = misc_feature - TaAHA_2BL_4AEA2109C_Triticum_aestivum
MWNPLSWVMEMAAIMAIALANGGGKPPDWQDFVGIIVLLVINSTISFIEENNAGNAAAALMANLAPKTKVLRDGRWGEQEASILVPGDIVSIKLGDIVPADARLLEGDPLKIDQSGLTGESLPVTKNPGDEVFSGSTCKQGEIEAVVIATGVHTFFGKAAHLVDSTNQVGHFQQVLTAIGNFCIVSIAVGIVIEIIVMFPIQRRKYRAGIENLLVLLIGGIPIAMPTVLSVTMAIGSHKLSQQGAITKRMTAIEELAGMDVLCSDKTGTLTLNKLSVDKNLVEVFAKGVDKEHVLLLAARASRVENQDAIDACMVGMLADPKEARAGIREVHFLPFNPTDKRTALTYIDAEGNWHRASKGAPEQIITLCNCKEDVKRKVHSVIEKYAERGLRSLAVARQEVPEKSKDSPGGPWQFIGLLPLFDPPRHDSAETIRKALVLGVNVKMITGDQLAIGKETGRRLGMGTNMYPSSALLGQSKDGSLESLPVDELIEKADGFAGVFPEHKYEIVKRLQEKKHIVGMTGDGVNDAPALKKADIGIAVDDATDAARSASDIVLTEPGLSVIISAVLTSRCIFQRMKNYTIYAVSITIRIVLGFMLIALIWKFDFAPFMVLIIAILNDGTIMTISKDRVKPSPLPDSWKLNEIFATGVVLGTYLALMTVVFFWVIHKTDFFTNKFGVRSIRDSEFEMMSALYLQVSIVSQALIFVTRSRSWSFVERPGFLLVTAFLLAQLVATLIAVYANWEFARIKGIGWGWAGVIWLFSIVFYFPLDIFKFFIRFVLSGRAWDNLLQNKTAFTTKENYGKGEREAQWATAQRTLHGLQAPEPASHTLFNDKSSYRELSEIAEQAKRRAEIARLRELNTLKGHVESVVKLKGLDIDT

SEQ ID NO: 15 - protein, 951 aa, Triticum aestivum
  REGION 1..951: note = misc_feature - TaAHA_2AL_2C50DEE4C.1_Triticum_aestivum
MGGLEEIRNEAVDLENIPIEEVFEQLKCTRQGLTSDEGAQRVEIFGLNKLEEKKESKVLKFLGFMWNPLSWVMEMAAIMAIALANGGGKPPDWQDFVGIIVLLVINSTISFIEENNAGNAAAALMANLAPKTKVLRDGRWGEQEASILVPGDIVSIKLGDIVPADARLLEGDPLKIDQSGLTGESLPVTKNPGDEVFSGSTCKQGEIEAVVIATGVHTFFGKAAHLVDSTNQVGHFQQVLTAIGNFCIVSIAVGIVIEIIVMFPIQRRKYRAGIENLLVLLIGGIPIAMPTVLSVTMAIGSHKLSQQGAITKRMTAIEELAGMDVLCSDKTGTLTLNKLSVDKNLVEVFAKGVDKEHVLLLAARASRVENQDAIDACMVGMLADPKEARAGIREVHFLPFNPTDKRTALTYIDAEGNWHRASKGAPEQIITLCNCKEDVKRKVHSVIEKYAERGLRSLAVARQEVPEKSKDSPGGPWQFIGLLPLFDPPRHDSAETIRKALVLGVNVKMITGDQLAIGKETGRRLGMGTNMYPSSALLGQSKDGSLESLPVDELIEKADGFAGVFPEHKYEIVKRLQEKKHIVGMTGDGVNDAPALKKADIGIAVDDATDAARSASDIVLTEPGLSVIISAVLTSRCIFQRMKNYTIYAVSITIRIVLGFMLIALIWKFDFAPFMVLIIAILNDGTIMTISKDRVKPSPLPDSWKLNEIFATGVVLGTYLALVTVVFFWLIHKTDFFTNKFGVESIRNTEFKEMSALYLQVSIVSQALIFVTRSRSWSFVERPGFLLVTAFLLAQLVATLIAVYANWDFARIKGIGWGWAGVIWLFSIVFYFPLDIFKFFIRFVLSGRAWDNLLQNKTAFTTKENYGKGEREAQWATAQRTLHGLQAPEPASHTLFNDKSSYRELSEIAEQAKRRAEIARLRELNTLKGHVESVVKLKGLDIDTINQNYTV

SEQ ID NO: 16 - protein, 951 aa, Triticum aestivum
  REGION 1..951: note = misc_feature - TaAHA_Q5PSM6_Triticum_aestivum
MGGLEEIRNEAVDLENIPIEEVFEQLKCTRQGLTSDEGAQRVEIFGLNKLEEKKESKVLKFLGFMWNPLSWVMEMAAIMAIALANGGGKPPDWQDFVGIIVLLVINSTISFIEENNAGNAAAALMANLAPKTKVLRDGRWGEQGASILVPGDIVSIKLGDIVPADARLLEGDPLKIDQSGLTGESLPVTKNPGDEVFSGSTCKQGEIEAVVIATGVRTFFGKAAHLVDSTNQVGHFQQVLTAIGNFCIVSIAVGIVIEIIVMFPIQRRKYRAGIENLLVLLIGGIPIAMPTVLSVTMAIGSHKLSQQGAITKRMTAIEELAGMDVLCSDKTGTLTLNKLSVDKNLVEVFAKGVDKEHVLLLAARASRVENQDAIDACMVGMLADPKEARAGIREVHFLPFNPTDKRTALTYIDAEGNWHRASKGAPEQIITLCNCKEDVKRKVHSVIEKYAERGLRSLAVARQEVPEKSKDSPGGPWQFIGLLPLFDPPRHDSAETIRKALVLGVNVKMITGDQLAIGKETGRRLGMGTNMYPSSALLGQSKDGSLESLPVDELIEKADGFAGVFPEHKYEIVKRLQEKKHIVGMTGDGVNDAPALKKADIGIAVDDATDAARSASDIVLTEPGLSVIISAVLTSRCIFQRMKNYTIYAVSITIRIVLGFMLIALIWKFDFAPFMVLIIAILNDGTIMTISKDRVKPSPLPDSWKLNEIFATGVVLGTYLALVTVVFFWLIHKTDFFTNKFGVESIRNTEFKEMSALYLQVSIVSQALIFVTRSRSWSFVERPGFLLVTAFLLAQLVATLIAVYANWDFARIKGIGWGWAGVIWLFSIVFYFPLDIFKFFIRFVLSGRAWDNLLQNKTAFTTKENYGKGEREAQWATAQRTLHGLQAPEPASHTLFNDKSSYRELSEIAEQAKRRAEIARLRELNTLKSHVESVVKLKGLDIDTINQNYTV

SEQ ID NO: 17 - protein, 931 aa, Capsella rubella
  REGION 1..931: note = misc_feature - CrAHA|XP_006293643_Capsella_rubella
MEEVFEELKCTKQGLTREEASHRLDLFGPNKLEEKKESKVLKFLGFMWNPLSWVMEAAALMAIALANGGGRPPDWQDFVGIVCLLFINSTISFIEENNAGNAAAALMAGLAPKTKVLRDNQWSEQEASILVPGDVISVKLGDIIPADARLLDGDPLKIDQSSLTGESIPVTKNPGDEVFSGSTCKQGEIEAIVVATGVHTFFGKAAHLVDSTNQIGHFQKVLTSIGNFCICSIGLGIIVELIVMYPIQRRKYRDGIDNLLVLLIGGIPIAMPTVLSVTMAIGSHRLSKQGAITKRMTAIEEMAGMDVLCSDKTGTLTLNRLTVDRNLVEVFAKGVGKEHVFLLAARASRIENQDAIDAAIVGMLGDPKEARAGVREVHFFPFNPVDKRTALTYIDSDGNWHRASKGAPEQILNLCNCKEDVRRKVHGVIDKFAERGLRSLAVARQKVPEKKKDASGDPWQLVGLLPLFDPPRHDSAETIRRALNLGVNVKMITGDQLAIGKETGRRLGMGTNMYPSSALLGQEKDSSLGALPVDELIEKADGFAGVFPEHKYEIVNRLQQRKHICGMTGDGVNDAPALKKADIGIAVADATDAARGASDIVLTEPGLSVIISAVLTSRAIFQRMKNYTIYAVSITIRIVFGFMFIALIWQFDFSPFMVLIIAILNDGTIMTISKDRVKPSPQPDSWKLREIFSTGVVLGGYQALMTVVFFWVMKDTDIFSNMLGVRPLSQRPEQMMAALYLQVSIISQALIFVTRSRSWSFVERPGLLLLGAFVIAQLVATFIAVYANWSFARIEGAGWGWAGVIWLYSLITYIPLDLLKFGIRYVLSGKAWLNLLENKTAFTTKKDYGKEEREAQWAAAQRTLHGLQPAETNNIFNEKNSYHDLSQIAEQAKRRAEVVRLREINTLKGHVESVVKLKGLDIDTIQQHYTV

SEQ ID NO: 18 - protein, 957 aa, Camelina sativa
  REGION 1..957: note = misc_feature - CsAHA|XP_010472461_Camelina_sativa
MAAAAGKGNELDHIKNESVDLVRIPMEEVFEELKCTKQGLTTDEASHRLDLFGPNKLEEKKESKLLKFLGFMWNPLSWVMEAAALMAIALANGGGRPPDWQDFVGIVCLLFINSTISFIEENNAGNAAAALMAGLAPKTKVLRDNQWSEQEASILVPGDVISLKLGDIIPADARLLDGDPLKIDQSSLTGESIPVTKNPGDEVFSGSTCKQGEIEAIVVATGVHTFFGKAAHLVDSTNQIGHFQKVLTSIGNFCICSIALGIVVELLVMYPIQRRGYRDGIDNLLVLLIGGIPIAMPTVLSVTMAIGSHRLSKQGAITKRMTAIEEMAGMDVLCSDKTGTLTLNKLTVDRNLVEVFAKGVGKEHVFLLAARASRIENQDAIDAAIVGMLADPKEARAGVREVHFFPFNPVDKRTALTYVDYSDGNWYRASKGAPEQILNLCDCKEDVRRKVHGVIDKFAERGLRSLAVARQEVPEKKKDASGGPWQLVGLLPLFDPPRHDSAETIRRALNLGVNVKMITGDQLAIGKETGRRLGMGTNMYPSSALLGQEKDSSLGALPVDELIEKADGFAGVFPEHKYEIVNRLQQRKHICGMTGDGVNDAPALKKADIGIAVADSTDAARGASDIVLTEPGLSVIISAVLTSRAIFQRMKNYTIYAVSITIRIVFGFMFIALIWQFDFSPFMVLIIAILNDGTIMTISKDRVKPSPQPDSWKLREIFSTGVVLGGYQAFMTVVFFWVMKDTNIFSNMLGVRPLSQRPEQMMAALYLQVSIISQALIFVTRSRSWSFVERPGLLLLGAFVIAQLVATFIAVYADWSFARIEGAGWGWAGVIWLYSLITYIPLDLLKFGIRYVLSGKAWLNLLENKTAFTTKKDYGKEEREAQWAAAQRTLHGLQPAETNNIFNEKNSYSELSQIAEQAKRRAEVVRLREINSLKGHVESVVKLKGLDIDTIQQHYTV

SEQ ID NO: 19 - protein, 951 aa, Medicago truncatula
  REGION 1..951: note = misc_feature - MtAHA|XP_013465721_Medicago_truncatula
MGGISLEEIKNESVDLERIPIDEVFEQLKCSRQGLTSEEGANRLQVFGPNKLEEKKESKFLKFLGFMWNPLSWVMEAAAIMAIALANGSGRPPDWQDFVGIIALLVINSTISFIEENNAGNAAAALMAGLAPKTKVLRDSRWSEQDAAILVPGDIISIKLGDIVPADARLLEGDPLSIDQSALTGESLPVTKSSSDEVFSGSTVKKGEIEAVVIATGVHTFFGKAAHLVDSTNQVGHFQKVLTAIGNFCICSIAIGILVELIVMYPIQHRKYRDGIDNLLVLLIGGIPIAMPTVLSVTMAIGSHRLSQQGAITKRMTAIEEMAGMDVLCSDKTGTLTLNKLSVDRNLIEVFAKGIEKEYVMLLAARASRTENQDAIDAAIVGMLADPKEARAGIREVHFLPFNPVDKRTALTYIDSDGKWHRASKGAPEQIINLCNCKEDVRKKVHAVIDKFAERGLRSLGVARQEVPERTKDSPGGPWQFVGLLPLFDPPRHDSAETIRRALNLGVNVKMITGDQLAIGKETGRRLGMGTNMYPSSALLGQDKDSSISALPVDELIEKADGFAGVFPEHKYEIVKRLQEKKHICGMTGDGVNDAPALKKADIGIAVADATDAARSASDIVLTEPGLSVIISAVLTSRAIFQRMKNYTIYAVSITIRIVFGFMFIALIWKFDFAPFMVLIIAILNDGTIMTISKDRVKPSPLPDSWKLREIFATGVVLGSYMALMTVVFFWAMKDTNFFSNKFGVRPIRHNPDEMMAALYLQVSIISQALIFVTRSRSWSFAERPGLLLLGAFLIAQLIATFIAVYANWGFARIKGMGWGWAGVIWLYSLVTYIPLDLLKFAIRYFLSGKAWDNLLENKTAFTTKKDYGKEEREAQWAAAQRTLHGLQPPEASNVFNEKNSYRELSEIAEQAKRRAEVARLREVHTLKGHVESVVKLKGLDIDTIQQHYTV

SEQ ID NO: 20 - protein, 955 aa, Populus euphratica
  REGION 1..955: note = misc_feature - PeAHA|XP_011031360_Populus_euphratica
MSSKGGISLEEIKNESVDLERIPMEEVFEQLKCTREGLSADEGASRLQVFGPNKLEEKKESKILKFLGFMWNPLSWVMEAAALMAIALANGDGRPPDWQDFVGIVVLLVINSTISFIEENNAGNAAAALMAGLAPKTKVLRDGRWSEQDASILVPGDIISIKLGDIVPADARLLEGDPLKIDQSALTGESLPVTKNPSDEVFSGSTCKQGEIEAVVIATGVHTFFGKAAHLVDSTNQVGHFQKVLTAIGNFCICSIAVGIFAELIVMYPIQHRKYRDGIDNLLVLLIGGIPIAMPTVLSVTMAIGSHRLSQQGAITKRMTAIEEMAGMDVLCSDKTGTLTLNKLTVDRSLIEVFAKGVEKEHVILLAARASRTENQDAIDAAIVGMLADPKEARAGIREVHFLPFNPVDKRTALTYIDSDGNWHRASKGAPEQILTLCNCKEDVKKKVHSVIDKFAERGLRSLGVAKQEVPEKSKDAAGAPWQLVGLLPLFDPPRHDSAETIRRALHLGVNVKMITGDQLAIGKETGRRLGMGTNMYPSSALLGQDRDASIAALPVDELIEKADGFAGVFPEHKYEIVKRLQDRKHICGMTGDGVNDAPALKKADIGIAVADATDAARSASDIVLTEPGLSVIISAVLTSRAIFQRMKNYTIYAVSITIRIVFGFMFIALIWKFDFAPFMVLIIAILNDGTIMTISKDRVKPSPQPDSWKLKEIFSTGIVLGGYMALMTVLFFWIMKDTDFFSDKFGVRSLRKNDEEMMAALYLQVSIVSQALIFVTRSRSWSFVERPGFLLLGAFVAAQLIATLIAVYANWGFARIKGCGWGWAGVIWLFSVVTYVPLDILKFAIRYILSGKAWDNLLENKTAFTTKKDYGKEEREAQWATAQRTLHGLQPPETSHNMFSEKNSYRELSEIAEQAKRRAEMARLRELNTLKGHVESVVKLKGLDIDTIQQHYTV

SEQ ID NO: 21 - protein, 23 aa, synthetic construct
  REGION 1..23: note = Consensus sequence First transmembrane domain
  VARIANT 15: note = X is M, V or A
  VARIANT 18: note = X is L, I or V
  VARIANT 23: note = X is L or M
FLGFMWNPLSWVMEXAAXMAIAX

SEQ ID NO: 22 - protein, 23 aa, synthetic construct
  REGION 1..23: note = First transmembrane domain mutated at position 8 P->S
  VARIANT 15: note = X is M, V or A
  VARIANT 18: note = X is L, I or V
  VARIANT 23: note = X is L or M
FLGFMWNSLSWVMEXAAXMAIAX

SEQ ID NO: 23 - protein, 930 aa, Arabidopsis thaliana
  REGION 1..930: note = misc_feature - AtAHA5_P50S mutant
MEEVFEELKCTKQGLTANEASHRLDVFGPNKLEEKKESKLLKFLGFMWNSLSWVMEVAALMAIALANGGGRPPDWQDFVGIVCLLLINSTISFIEENNAGNAAAALMAGLAPKTKVLRDNQWSEQEASILVPGDVISIKLGDIIPADARLLDGDPLKIDQSSLTGESIPVTKNPSDEVFSGSICKQGEIEAIVIATGVHTFFGKAAHLVDNTNQIGHFQKVLTSIGNFCICSIALGIIVELLVMYPIQRRRYRDGIDNLLVLLIGGIPIAMPSVLSVTMATGSHRLFQQGAITKRMTAIEEMAGMDVLCCDKTGTLTLNKLTVDKNLVEVFAKGVGKEHVFLLAARASRIENQDAIDAAIVGMLADPKEARAGVREVHFFPFNPVDKRTALTYVDSDGNWHRASKGAPEQILNLCNCKEDVRRKVHGVIDKFAERGLRSLAVARQEVLEKKKDAPGGPWQLVGLLPLFDPPRHDSAETIRRALNLGVNVKMITGDQLAIGKETGRRLGMGTNMYPSSALLGQVKDSSLGALPVDELIEKADGFAGVFPEHKYEIVHRLQQRNHICGMTGDGVNDAPALKKADIGIAVVDATDAARGASDIVLTEPGLSVIISAVLTSRAIFQRMKNYTIYAVSITIRIVFGFMFIALIWQFDFSPFMVLIIAILNDGTIMTISKDRMKPSPQPDSWKLRDIFSTGVVLGGYQALMTVVFFWVMKDSDFFSNYFGVRPLSQRPEQMMAALYLQVSIISQALIFVTRSRSWSYAECPGLLLLGAFVIAQLVATFIAVYANWSFARIEGAGWGWAGVIWLYSFLTYIPLDLLKFGIRYVLSGKAWLNLLENKTAFTTKKDYGKEEREAQWAAAQRTLHGLQPAEKNNIFNEKNSYSELSQIAEQAKRRAEVVRLREINTLKGHVESVVKLKGLDIDTIQQHYT

SEQ ID NO: 24 - protein, 107 aa, synthetic construct
  REGION 1..107: note = Consensus sequence R1 domain
  VARIANT 4: note = X is R or K
  VARIANT 7: note = X is D, L, N or R
  VARIANT 9: note = X is L or M
  VARIANT 11: note = X is E, Q or D
  VARIANT 14: note = X is T or I
  VARIANT 18: note = X is T or S
  VARIANT 20: note = X is K or E
  VARIANT 24: note = X is K or R
  VARIANT 25: note = X is E or G
  VARIANT 33: note = X is T, A or L
  VARIANT 43: note = X is P or A
  VARIANT 44: note = X is P or A
  VARIANT 46: note = X is T, V, A, P, K or S
  VARIANT 47: note = X is A, S or N
  VARIANT 48: note = X is S, T or N
  VARIANT 49: note = X is N, S or H
  VARIANT 50: note = X is N or T
  VARIANT 51: note = X is I, V, M or L
  VARIANT 53: note = X is N, S or H
  VARIANT 54: note = X is E or D
  VARIANT 56: note = X is S or N
  VARIANT 59: note = X is R, S or H
  VARIANT 60: note = X is E or D
  VARIANT 74: note = X is I, V or M
  VARIANT 75: note = X is A or V
  VARIANT 80: note = X is L, I or V
  VARIANT 81: note = X is N, L or H
  VARIANT 82: note = X is T or S
  VARIANT 85: note = X is G or S
  VARIANT 99: note = X is D or E
  VARIANT 102: note = X is Q or N
  VARIANT 104: note = X is N or H
LSGXAWXNXLXNKXAFTXKXBYGXXEREAQWAXAQRTLHGLQXXEXXXXXXFXXKXSYXXLSZIAEQAKRRAEXXRLREXXXLKXHVESVVKLKGLDIXTIXQXYTV

SEQ ID NO: 25 - protein, 106 aa, synthetic construct
  REGION 1..106: note = Consensus sequence R1 domain
  VARIANT 4: note = X is R or K
  VARIANT 7: note = X is D, L, N or R
  VARIANT 9: note = X is L or M
  VARIANT 11: note = X is E, Q or D
  VARIANT 14: note = X is T or I
  VARIANT 18: note = X is T or S
  VARIANT 20: note = X is K or E
  VARIANT 24: note = X is K or R
  VARIANT 25: note = X is E or G
  VARIANT 33: note = X is T, A or L
  VARIANT 43: note = X is P or A
  VARIANT 44: note = X is P or A
  VARIANT 46: note = X is T, V, A, P, K or S
  VARIANT 47: note = X is S, T or N
  VARIANT 48: note = X is N, S or H
  VARIANT 49: note = X is N or T
  VARIANT 50: note = X is I, V, M or L
  VARIANT 52: note = X is N, S or H
  VARIANT 53: note = X is E or D
  VARIANT 55: note = X is S or N
  VARIANT 58: note = X is R, S or H
  VARIANT 59: note = X is E or D
  VARIANT 73: note = X is I, V or M
  VARIANT 74: note = X is A or V
  VARIANT 79: note = X is L, I or V
  VARIANT 80: note = X is N, L or H
  VARIANT 81: note = X is T or S
  VARIANT 84: note = X is G or S
  VARIANT 98: note = X is D or E
  VARIANT 101: note = X is Q or N
  VARIANT 103: note = X is N or H
LSGXAWXNXLXNKXAFTXKXBYGXXEREAQWAXAQRTLHGLQXXEXXXXXFXXKXSYXXLSZIAEQAKRRAEXXRLREXXXLKXHVESVVKLKGLDIXTIXQXYTV

SEQ ID NO: 26 - protein, 106 aa, synthetic construct; identical to SEQ ID NO: 25
  REGION 1..106: note = Consensus sequence R1 domain
  VARIANT 4: note = X is R or K
  VARIANT 7: note = X is D, L, N or R
  VARIANT 9: note = X is L or M
  VARIANT 11: note = X is E, Q or D
  VARIANT 14: note = X is T or I
  VARIANT 18: note = X is T or S
  VARIANT 20: note = X is K or E
  VARIANT 24: note = X is K or R
  VARIANT 25: note = X is E or G
  VARIANT 33: note = X is T, A or L
  VARIANT 43: note = X is P or A
  VARIANT 44: note = X is P or A
  VARIANT 46: note = X is T, V, A, P, K or S
  VARIANT 47: note = X is A, S or N
  VARIANT 48: note = X is N, S or H
  VARIANT 49: note = X is N or T
  VARIANT 50: note = X is I, V, M or L
  VARIANT 52: note = X is N, S or H
  VARIANT 53: note = X is E or D
  VARIANT 55: note = X is S or N
  VARIANT 58: note = X is R, S or H
  VARIANT 59: note = X is E or D
  VARIANT 73: note = X is I, V or M
  VARIANT 74: note = X is A or V
  VARIANT 79: note = X is L, I or V
  VARIANT 80: note = X is N, L or H
  VARIANT 81: note = X is T or S
  VARIANT 84: note = X is G or S
  VARIANT 98: note = X is D or E
  VARIANT 101: note = X is Q or N
  VARIANT 103: note = X is N or H

SEQ ID NO: 27 - protein, 105 aa, synthetic construct
  REGION 1..105: note = Consensus sequence R1 domain
  VARIANT 4: note = X is R or K
  VARIANT 7: note = X is D, L, N or R
  VARIANT 9: note = X is L or M
  VARIANT 11: note = X is E, Q or D
  VARIANT 14: note = X is T or I
  VARIANT 18: note = X is T or S
  VARIANT 20: note = X is K or E
  VARIANT 24: note = X is K or R
  VARIANT 25: note = X is E or G
  VARIANT 33: note = X is T, A or L
  VARIANT 43: note = X is P or A
  VARIANT 44: note = X is P or A
  VARIANT 46: note = X is T, V, A, P, K or S
  VARIANT 47: note = X is N, S or H
  VARIANT 48: note = X is N or T
  VARIANT 49: note = X is I, V, M or L
  VARIANT 51: note = X is N, S or H
  VARIANT 52: note = X is E or D
  VARIANT 54: note = X is S or N
  VARIANT 57: note = X is R, S or H
  VARIANT 58: note = X is E or D
  VARIANT 72: note = X is I, V or M
  VARIANT 73: note = X is A or V
  VARIANT 78: note = X is L, I or V
  VARIANT 79: note = X is N, L or H
  VARIANT 80: note = X is T or S
  VARIANT 83: note = X is G or S
  VARIANT 97: note = X is D or E
  VARIANT 100: note = X is Q or N
  VARIANT 102: note = X is N or H
LSGXAWXNXLXNKXAFTXKXBYGXXEREAQWAXAQRTLHGLQXXEXXXXFXXKXSYXXLSZIAEQAKRRAEXXRLREXXXLKXHVESVVKLKGLDIXTIXQXYTV

SEQ ID NO: 28 - protein, 107 aa, synthetic construct
  REGION 1..107: note = R1 domain mutated at position 31 W->L
  VARIANT 4: note = X is R or K
  VARIANT 7: note = X is D, L, N or R
  VARIANT 9: note = X is L or M
  VARIANT 11: note = X is E, Q or D
  VARIANT 14: note = X is T or I
  VARIANT 18: note = X is T or S
  VARIANT 20: note = X is K or E
  VARIANT 24: note = X is K or R
  VARIANT 25: note = X is E or G
  VARIANT 33: note = X is T, A or L
  VARIANT 43: note = X is P or A
  VARIANT 44: note = X is P or A
  VARIANT 46: note = X is T, V, A, P, K or S
  VARIANT 47: note = X is A, S or N
  VARIANT 48: note = X is S, T or N
  VARIANT 49: note = X is N, S or H
  VARIANT 50: note = X is N or T
  VARIANT 51: note = X is I, V, M or L
  VARIANT 53: note = X is N, S or H
  VARIANT 54: note = X is E or D
  VARIANT 56: note = X is S or N
  VARIANT 59: note = X is R, S or H
  VARIANT 60: note = X is E or D
  VARIANT 74: note = X is I, V or M
  VARIANT 75: note = X is A or V
  VARIANT 80: note = X is L, I or V
  VARIANT 81: note = X is N, L or H
  VARIANT 82: note = X is T or S
  VARIANT 85: note = X is G or S
  VARIANT 99: note = X is D or E
  VARIANT 102: note = X is Q or N
  VARIANT 104: note = X is N or H
LSGXAWXNXLXNKXAFTXKXBYGXXEREAQLAXAQRTLHGLQXXEXXXXXXFXXKXSYXXLSZIAEQAKRRAEXXRLREXXXLKXHVESVVKLKGLDIXTIXQXYTV

SEQ ID NO: 29 - protein, 107 aa, synthetic construct
  REGION 1..107: note = R1 domain mutated at position 31 W->A
  VARIANT 4: note = X is R or K
  VARIANT 7: note = X is D, L, N or R
  VARIANT 9: note = X is L or M
  VARIANT 11: note = X is E, Q or D
  VARIANT 14: note = X is T or I
  VARIANT 18: note = X is T or S
  VARIANT 20: note = X is K or E
  VARIANT 24: note = X is K or R
  VARIANT 25: note = X is E or G
  VARIANT 33: note = X is T, A or L
  VARIANT 43: note = X is P or A
  VARIANT 44: note = X is P or A
  VARIANT 46: note = X is T, V, A, P, K or S
  VARIANT 47: note = X is A, S or N
  VARIANT 48: note = X is S, T or N
  VARIANT 49: note = X is N, S or H
  VARIANT 50: note = X is N or T
  VARIANT 51: note = X is I, V, M or L
  VARIANT 53: note = X is N, S or H
  VARIANT 54: note = X is E or D
  VARIANT 56: note = X is S or N
  VARIANT 59: note = X is R, S or H
  VARIANT 60: note = X is E or D
  VARIANT 74: note = X is I, V or M
  VARIANT 75: note = X is A or V
  VARIANT 80: note = X is L, I or V
  VARIANT 81: note = X is N, L or H
  VARIANT 82: note = X is T or S
  VARIANT 85: note = X is G or S
  VARIANT 99: note = X is D or E
  VARIANT 102: note = X is Q or N
  VARIANT 104: note = X is N or H
LSGXAWXNXLXNKXAFTXKXBYGXXEREAQAAXAQRTLHGLQXXEXXXXXXFXXKXSYXXLSZIAEQAKRRAEXXRLREXXXLKXHVESVVKLKGLDIXTIXQXYTV

SEQ ID NO: 30 - protein, 931 aa, Arabidopsis thaliana
  REGION 1..931: note = misc_feature - AtAHA5_W857L mutant
MEEVFEELKCTKQGLTANEASHRLDVFGPNKLEEKKESKLLKFLGFMWNPLSWVMEVAALMAIALANGGGRPPDWQDFVGIVCLLLINSTISFIEENNAGNAAAALMAGLAPKTKVLRDNQWSEQEASILVPGDVISIKLGDIIPADARLLDGDPLKIDQSSLTGESIPVTKNPSDEVFSGSICKQGEIEAIVIATGVHTFFGKAAHLVDNTNQIGHFQKVLTSIGNFCICSIALGIIVELLVMYPIQRRRYRDGIDNLLVLLIGGIPIAMPSVLSVTMATGSHRLFQQGAITKRMTAIEEMAGMDVLCCDKTGTLTLNKLTVDKNLVEVFAKGVGKEHVFLLAARASRIENQDAIDAAIVGMLADPKEARAGVREVHFFPFNPVDKRTALTYVDSDGNWHRASKGAPEQILNLCNCKEDVRRKVHGVIDKFAERGLRSLAVARQEVLEKKKDAPGGPWQLVGLLPLFDPPRHDSAETIRRALNLGVNVKMITGDQLAIGKETGRRLGMGTNMYPSSALLGQVKDSSLGALPVDELIEKADGFAGVFPEHKYEIVHRLQQRNHICGMTGDGVNDAPALKKADIGIAVVDATDAARGASDIVLTEPGLSVIISAVLTSRAIFQRMKNYTIYAVSITIRIVFGFMFIALIWQFDFSPFMVLIIAILNDGTIMTISKDRMKPSPQPDSWKLRDIFSTGVVLGGYQALMTVVFFWVMKDSDFFSNYFGVRPLSQRPEQMMAALYLQVSIISQALIFVTRSRSWSYAECPGLLLLGAFVIAQLVATFIAVYANWSFARIEGAGWGWAGVIWLYSFLTYIPLDLLKFGIRYVLSGKAWLNLLENKTAFTTKKDYGKEEREAQLAAAQRTLHGLQPAEKNNIFNEKNSYSELSQIAEQAKRRAEVVRLREINTLKGHVESVVKLKGLDIDTIQQHYTV

SEQ ID NO: 31 - protein, 961 aa, Zea mays
  REGION 1..961: note = misc_feature - ZmAHA5_W885L mutant
MGPLQRRPTAMGGLEEIKNEAVDLENIPIEEVFEQLKCTREGLSSSEGQQRLEIFGPNRLEEKKESKILKFLGFMWNPLSWVMEMAAVMAIALANGGGKPPDWEDFVGIIVLLVINSTISFIEENNAGNAAAALMANLAPKTKVLRDGRWGEQEAAILVPGDIVSIKLGDIVPADARLLEGDPLKVDQSALTGESLPVTKGPGDEVFSGSTCKQGEIEAVVIATGVHTFFGKAAHLVDSTNQVGHFQQVLTAIGNFCICSIAVGIVVEIIVMFPIQHRRYRSGIENLLVLLIGGIPIAMPTVLSVTMAIGSHKLSQQGAITKRMTAIEEMAGMDVLCSDKTGTLTLNKLSVDKNLVEVFCKGVDKDHVLLLAARASRTENQDAIDAAMVGMLADPKEARAGIREIHFLPFNPVDKRTALTYIDADGHWHRVSKGAPEQILDLCHCKEDLRRKVHGIIDKYAERGLRSLAVARQEVPERNKESPGGPWQFVGLLPLFDPPRHDSAETIRKALVLGVNVKMITGDQLAIGKETGRRLGMGTNMYPSSALLGQNKDATLEALPVDELIEKADGFAGVFPEHKYEIVKRLQEKKHIVGMTGDGVNDAPALKKADIGIAVADATDAARSASDIVLTEPGLSVIISAVLTSRCIFQRMKNYTIYAVSITIRIVLGFMLIALIWQYDFSPFMVLIIAILNDGTIMTISKDRVKPSPLPDSWKLKEIFATGIVLGSYLALMTVIFFWAMHKTDFFSDKFGVRSIRDSEHEMMSALYLQVSIVSQALIFVTRSRSWSFVERPGLLLVTAFLLAQLVATFLAVYANWGFARIKGIGWGWAGVVWLYSIVFYFPLDLIKFFIRFVLSGRAWDNLLENKTAFTTKKDYGREEREAQLATAQRTLHGLQPPEAATSTLFHDKNSYRELSEIAEQAKRRAEIARLRELNTLKGHVESVVKLKGLDIDTIQQNYTV

SEQ ID NO: 32 - DNA, 40 bp, synthetic construct
  misc_feature 1..40: note = Amplification primer for the AtAHA5 gene
  source 1..40: mol_type = other DNA
ggattctagaactagtatggaggaagtgttcgaggagctg

SEQ ID NO: 33 - DNA, 42 bp, synthetic construct
  misc_feature 1..42: note = Amplification primer for the AtAHA5 gene
  source 1..42: mol_type = other DNA
cggtatcataagcttgttaaacggtgtaatgttgctgaatcg

SEQ ID NO: 34 - DNA, 27 bp, synthetic construct
  misc_feature 1..27: note = Primers for site directed mutagenesis of P50S
  source 1..27: mol_type = other DNA
gggttcatgtggaactcattgtcgtgg

SEQ ID NO: 35 - DNA, 27 bp, synthetic construct
  misc_feature 1..27: note = Primers for site directed mutagenesis of P50S
  source 1..27: mol_type = other DNA
ccacgacaatgagttccacatgaaccc

SEQ ID NO: 36 - DNA, 29 bp, synthetic construct
  misc_feature 1..29: note = Primers for site directed mutagenesis of W857L
  source 1..29: mol_type = other DNA
ggaaagagaagctcaattggctgcagctc

SEQ ID NO: 37 - DNA, 29 bp, synthetic construct
  misc_feature 1..29: note = Primers for site directed mutagenesis of W857L
  source 1..29: mol_type = other DNA
gagctgcagccaattgagcttctctttcc

SEQ ID NO: 38 - DNA, 48 bp, synthetic construct
  misc_feature 1..48: note = Amplification primer for the promoter pAtAHA5
  source 1..48: mol_type = other DNA
ggggacaactttgtatagaaaagttgcaaccatcatgacaagcgtctg

SEQ ID NO: 39 - DNA, 52 bp, synthetic construct
  misc_feature 1..52: note = Amplification primer for the promoter pAtAHA5
  source 1..52: mol_type = other DNA
ggggactgcttttttgtacaaacttggggtattcgtacctagacccatcatt

SEQ ID NO: 40 - DNA, 922 bp, Sorghum bicolor
  misc_feature 1..922: note = proSbMYB60
  source 1..922: mol_type = genomic DNA
ctccgtcgctagcgttgtaatttttatcagcctctctccatctgattggatgagctgcttctcctagctagcacgttgtcaaccattggggacgccctttaggagtgaaagtgatgcagttactctactcccatccggtccaaatcccaacctaagggatatatgacgaagagttttcaatttcagtataatgtgtatagcaagtgatttgattttcctgcagattcttgagagagaagggccaaacctgcatatgtgccaggtgcgcactcacagtcacacacacggtcctcatcaatgcccaagaaatctggagagacacagacaaacgcaagcaattactccattcatgagcgccacagcagaaaaaggagtcctccagaaccggccgtgttcttggcttgcacacacacacacacacacgcgaaagcagcgcatgcaacaaagcaaagatcacacacggccggagcaagagagacgcccatattcatgtgatcacctggctgacttttgcattggaaagcaacagtgcgagatcaaaagcgaacgcagcacacagcacagcacagcacagggatcgagttgctctactccgtatatgaggtcatgcagtatataagcactcacatgcagctataccttagctagggcacctgtgcaaggggacaggtaggagataaagcaatgtctgctggctagaataaaaggaggccgatatcgtatcagcagagggttccaggggagaaggccgttctgcagggccgccgtaaaaggctgtgtaggaagcagccacagcttctcatatcttcttgttcttcgactacttgcctagctactatatctactagactagccctaggctgcttgctagctagtaggtgtatcgatcgtccgaggtaggaggagacagcaggaagcaggc

SEQ ID NO: 41 - DNA, 773 bp, Zea mays
  misc_feature 1..773: note = proZmMYB60
  source 1..773: mol_type = genomic DNA
ggaagtgttttgattttcctgcagttctcgatcgagagaggccctcttgaatctgaaagaggtcgagaagggccagcctgcgtgtcccaggagcgtgcgctcacacacacggtcctcatcaatgcccaagaaatctggagagacacaaacaaacgcaaagcaattactcacttcatgagcgccacagcagaaaaggagtcctcaagcagtacgtcaaccggccgtgttctcgcacacgcaaaagcagcgcatgcaacaaagcaaagatcacacacccggagcgaagagacgcccattcatgtgatcacctggctgacttttgcattggaaagctacagtgcgagatcaaaagcgaacgcagcacagcacaggccgggggagtggcttatatgaggtcatgcagtatataagcactcacatgcagctagctaggtagagcacctgtgcatcaaggtaaagcagatggggcctgctctgctgcctgcctgcctgctggcatgcgtgtacgtcctagaataaaaggaggcccgtatcgtattagcagagggttccagggagaagagaagggagggcgaggccactggctgtgtaggcagcagccagccacacagacacagcttcgttcccatctcttctcgttcttcatcacctacttgcctactatatctactagccacgctagctagactagccttagaaaaggctagctcactagctgcttgctaggtgtgtaccgatcgtccgaggtaggagacacagcacgcagcaggggt

SEQ ID NO: 42 - DNA, 674 bp, synthetic construct
  misc_feature 1..674: note = pro35S
  source 1..674: mol_type = other DNA
attgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggac

SEQ ID NO: 43 - DNA, 4023 bp, synthetic construct
  misc_feature 1..4023: note = Cassette 35S-AtAHA5P50S
  source 1..4023: mol_type = other DNA
attgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggtaccgggccccccctcgaggcgcgccaaataatgattttattttgactgatagtgacctgttcgttgcaacaaattgatgagcaatgcttttttataatgccaactttgtacaaaaaagcaggctccatggaggaagtgttcgaggagctgaaatgtacaaaacaaggtcttacggcaaatgaagcttctcaccgtcttgacgtttttggtcctaacaagcttgaagagaagaaggaaagcaagttacttaagtttttagggttcatgtggaactcattgtcgtgggtgatggaggtggctgcactaatggcaatcgctttggccaacggaggaggacggccaccggattggcaagactttgtggggatagtgtgtttactattaatcaactcaaccattagtttcattgaggaaaataacgccggtaatgctgcagccgctctcatggccggtctagcccctaagacaaaggttctcagagataatcaatggagcgagcaagaagcatcgattctcgtccccggagatgtaatcagcatcaagctcggcgatatcattcccgccgatgcacgtttactcgacggcgatcctctcaaaatcgatcaatcatcactcaccggtgaatcaatccccgtcacaaaaaaccccagcgatgaagtcttctccggctcaatatgcaaacaaggcgaaatcgaagcaatcgtcatcgccactggagtccacacattcttcggcaaagcagctcatctcgtcgacaacacaaaccaaatcggccatttccagaaagttctcacttcgattggtaacttctgtatctgttcgattgctttaggaatcatcgttgagcttttagtaatgtatccgattcaacgccggagatatcgagacggaatcgataatttacttgttttgttgatcggagggattcctatagctatgcctagtgtgttatctgttacaatggcgactggttctcatagattgtttcaacaaggagctattactaagagaatgactgctattgaggaaatggctggtatggatgtattgtgttgtgataagactggtactcttacgcttaataagcttacggttgataagaacttagtcgaggttttcgctaaaggagttggtaaagaacatgttttccttttggctgcgagagcttcgaggattgagaatcaagatgcgattgatgctgctattgttggaatgcttgctgatcctaaagaggctagagctggtgttagagaggttcattttttcccttttaatccggttgataagagaactgctttgacttatgttgactctgatggaaactggcatagagctagtaaaggagctccagagcagatactgaacctctgtaattgcaaggaagatgttaggaggaaagttcatggagtgattgataagtttgctgagcgtggacttcgctctttggctgttgcaagacaggaagttctcgagaagaagaaagatgctcctggtggcccatggcaacttgtaggtcttttacctctttttgatcctccgaggcatgacagtgccgagacaatcaggagggctttaaacctcggtgttaatgttaagatgataaccggggaccagcttgcaattgggaaagagaccggtcgaaggcttggaatgggtacgaacatgtatccttcttctgcgctgctcggacaagtcaaagattcttccttgggtgcacttcctgtggatgaactgatagagaaggctgatgggtttgcaggagtctttccagaacataagtatgagattgttcataggctgcaacaaaggaatcacatatgtggcatgactggagatggtgtgaatgatgccccggctctcaagaaggcggatattggtatagctgttgttgatgctactgatgctgcgagaggcgcttctgatatcgttctcacagaacccggattgagtgttatcattagtgctgtactcaccagtagagccatattccaaagaatgaaaaattacactatttatgcggtttcaatcacgatacgaattgtgtttggcttcatgttcattgccctcatatggcagttcgatttttcgcctttcatggttctgatcatagcaatcttaaacgacggaacaatcatgacaatatcaaaagacagaatgaagccatctccacagccagatagctggaaactcagagatatattctcgaccggcgtcgtgcttggaggttaccaggccttgatgacagtcgttttcttctgggtgatgaaagacagtgattttttctcgaactattttggtgtgagaccactgagccaacgtcctgaacaaatgatggctgctctgtatctacaagttagcatcataagccaggctctcatcttcgtcaccagatcccgtagctggtcctacgccgaatgccctggccttctcttacttggtgcatttgtcatagctcagctggtggcgacatttatagcagtttatgcaaactggagttttgcccggatagaaggagccggttggggatgggctggagtgatctggctatacagtttcctaacgtacatccctcttgacttactcaagtttggaatccgctatgttttgagtggaaaagcttggttaaatcttcttgagaacaagactgccttcacgacgaagaaagactatgggaaagaggaaagagaagctcaatgggctgcagctcaaagaaccctccacggacttcaaccagcagagaaaaacaacatctttaacgaaaagaatagttacagtgaactctctcagattgctgaacaggctaaacgccgagctgaggttgtcaggctgagagagataaatacattaaaagggcatgtagagtcagtggtgaagcttaaaggacttgacattgacacgattcagcaacattacaccgtttaaaacccagctttcttgtacaaagttggcattataagaaagcattgcttatcaatttgttgcaacgaacaggtcactatcagtcaaaataaaatcattatttgttaattaactagttctagagcggccgcccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttact

SEQ ID NO: 44 - DNA, 4023 bp, synthetic construct
  misc_feature 1..4023: note = Cassette 35S-AtAHA5W857L
  source 1..4023: mol_type = other DNA
attgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggtaccgggccccccctcgaggcgcgccaaataatgattttattttgactgatagtgacctgttcgttgcaacaaattgatgagcaatgcttttttataatgccaactttgtacaaaaaagcaggctccatggaggaagtgttcgaggagctgaaatgtacaaaacaaggtcttacggcaaatgaagcttctcaccgtcttgacgtttttggtcctaacaagcttgaagagaagaaggaaagcaagttacttaagtttttagggttcatgtggaacccattgtcgtgggtgatggaggtggctgcactaatggcaatcgctttggccaacggaggaggacggccaccggattggcaagactttgtggggatagtgtgtttactattaatcaactcaaccattagtttcattgaggaaaataacgccggtaatgctgcagccgctctcatggccggtctagcccctaagacaaaggttctcagagataatcaatggagcgagcaagaagcatcgattctcgtccccggagatgtaatcagcatcaagctcggcgatatcattcccgccgatgcacgtttactcgacggcgatcctctcaaaatcgatcaatcatcactcaccggtgaatcaatccccgtcacaaaaaaccccagcgatgaagtcttctccggctcaatatgcaaacaaggcgaaatcgaagcaatcgtcatcgccactggagtccacacattcttcggcaaagcagctcatctcgtcgacaacacaaaccaaatcggccatttccagaaagttctcacttcgattggtaacttctgtatctgttcgattgctttaggaatcatcgttgagcttttagtaatgtatccgattcaacgccggagatatcgagacggaatcgataatttacttgttttgttgatcggagggattcctatagctatgcctagtgtgttatctgttacaatggcgactggttctcatagattgtttcaacaaggagctattactaagagaatgactgctattgaggaaatggctggtatggatgtattgtgttgtgataagactggtactcttacgcttaataagcttacggttgataagaacttagtcgaggttttcgctaaaggagttggtaaagaacatgttttccttttggctgcgagagcttcgaggattgagaatcaagatgcgattgatgctgctattgttggaatgcttgctgatcctaaagaggctagagctggtgttagagaggttcattttttcccttttaatccggttgataagagaactgctttgacttatgttgactctgatggaaactggcatagagctagtaaaggagctccagagcagatactgaacctctgtaattgcaaggaagatgttaggaggaaagttcatggagtgattgataagtttgctgagcgtggacttcgctctttggctgttgcaagacaggaagttctcgagaagaagaaagatgctcctggtggcccatggcaacttgtaggtcttttacctctttttgatcctccgaggcatgacagtgccgagacaatcaggagggctttaaacctcggtgttaatgttaagatgataaccggggaccagcttgcaattgggaaagagaccggtcgaaggcttggaatgggtacgaacatgtatccttcttctgcgctgctcggacaagtcaaagattcttccttgggtgcacttcctgtggatgaactgatagagaaggctgatgggtttgcaggagtctttccagaacataagtatgagattgttcataggctgcaacaaaggaatcacatatgtggcatgactggagatggtgtgaatgatgccccggctctcaagaaggcggatattggtatagctgttgttgatgctactgatgctgcgagaggcgcttctgatatcgttctcacagaacccggattgagtgttatcattagtgctgtactcaccagtagagccatattccaaagaatgaaaaattacactatttatgcggtttcaatcacgatacgaattgtgtttggcttcatgttcattgccctcatatggcagttcgatttttcgcctttcatggttctgatcatagcaatcttaaacgacggaacaatcatgacaatatcaaaagacagaatgaagccatctccacagccagatagctggaaactcagagatatattctcgaccggcgtcgtgcttggaggttaccaggccttgatgacagtcgttttcttctgggtgatgaaagacagtgattttttctcgaactattttggtgtgagaccactgagccaacgtcctgaacaaatgatggctgctctgtatctacaagttagcatcataagccaggctctcatcttcgtcaccagatcccgtagctggtcctacgccgaatgccctggccttctcttacttggtgcatttgtcatagctcagctggtggcgacatttatagcagtttatgcaaactggagttttgcccggatagaaggagccggttggggatgggctggagtgatctggctatacagtttcctaacgtacatccctcttgacttactcaagtttggaatccgctatgttttgagtggaaaagcttggttaaatcttcttgagaacaagactgccttcacgacgaagaaagactatgggaaagaggaaagagaagctcaattggctgcagctcaaagaaccctccacggacttcaaccagcagagaaaaacaacatctttaacgaaaagaatagttacagtgaactctctcagattgctgaacaggctaaacgccgagctgaggttgtcaggctgagagagataaatacattaaaagggcatgtagagtcagtggtgaagcttaaaggacttgacattgacacgattcagcaacattacaccgtttaaaacccagctttcttgtacaaagttggcattataagaaagcattgcttatcaatttgttgcaacgaacaggtcactatcagtcaaaataaaatcattatttgttaattaactagttctagagcggccgcccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttact

SEQ ID NO: 45 - DNA, 4023 bp, synthetic construct
  misc_feature 1..4023: note = Cassette 35S-AtAHA5 WT
  source 1..4023: mol_type = other DNA
attgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccccgggtaccgggccccccctcgaggcgcgccaaataatgattttattttgactgatagtgacctgttcgttgcaacaaattgatgagcaatgcttttttataatgccaactttgtacaaaaaagcaggctccatggaggaagtgttcgaggagctgaaatgtacaaaacaaggtcttacggcaaatgaagcttctcaccgtcttgacgtttttggtcctaacaagcttgaagagaagaaggaaagcaagttacttaagtttttagggttcatgtggaacccattgtcgtgggtgatggaggtggctgcactaatggcaatcgctttggccaacggaggaggacggccaccggattggcaagactttgtggggatagtgtgtttactattaatcaactcaaccattagtttcattgaggaaaataacgccggtaatgctgcagccgctctcatggccggtctagcccctaagacaaaggttctcagagataatcaatggagcgagcaagaagcatcgattctcgtccccggagatgtaatcagcatcaagctcggcgatatcattcccgccgatgcacgtttactcgacggcgatcctctcaaaatcgatcaatcatcactcaccggtgaatcaatccccgtcacaaaaaaccccagcgatgaagtcttctccggctcaatatgcaaacaaggcgaaatcgaagcaatcgtcatcgccactggagtccacacattcttcggcaaagcagctcatctcgtcgacaacacaaaccaaatcggccatttccagaaagttctcacttcgattggtaacttctgtatctgttcgattgctttaggaatcatcgttgagcttttagtaatgtatccgattcaacgccggagatatcgagacggaatcgataatttacttgttttgttgatcggagggattcctatagctatgcctagtgtgttatctgttacaatggcgactggttctcatagattgtttcaacaaggagctattactaagagaatgactgctattgaggaaatggctggtatggatgtattgtgttgtgataagactggtactcttacgcttaataagcttacggttgataagaacttagtcgaggttttcgctaaaggagttggtaaagaacatgttttccttttggctgcgagagcttcgaggattgagaatcaagatgcgattgatgctgctattgttggaatgcttgctgatcctaaagaggctagagctggtgttagagaggttcattttttcccttttaatccggttgataagagaactgctttgacttatgttgactctgatggaaactggcatagagctagtaaaggagctccagagcagatactgaacctctgtaattgcaaggaagatgttaggaggaaagttcatggagtgattgataagtttgctgagcgtggacttcgctctttggctgttgcaagacaggaagttctcgagaagaagaaagatgctcctggtggcccatggcaacttgtaggtcttttacctctttttgatcctccgaggcatgacagtgccgagacaatcaggagggctttaaacctcggtgttaatgttaagatgataaccggggaccagcttgcaattgggaaagagaccggtcgaaggcttggaatgggtacgaacatgtatccttcttctgcgctgctcggacaagtcaaagattcttccttgggtgcacttcctgtggatgaactgatagagaaggctgatgggtttgcaggagtctttccagaacataagtatgagattgttcataggctgcaacaaaggaatcacatatgtggcatgactggagatggtgtgaatgatgccccggctctcaagaaggcggatattggtatagctgttgttgatgctactgatgctgcgagaggcgcttctgatatcgttctcacagaacccggattgagtgttatcattagtgctgtactcaccagtagagccatattccaaagaatgaaaaattacactatttatgcggtttcaatcacgatacgaattgtgtttggcttcatgttcattgccctcatatggcagttcgatttttcgcctttcatggttctgatcatagcaatcttaaacgacggaacaatcatgacaatatcaaaagacagaatgaagccatctccacagccagatagctggaaactcagagatatattctcgaccggcgtcgtgcttggaggttaccaggccttgatgacagtcgttttcttctgggtgatgaaagacagtgattttttctcgaactattttggtgtgagaccactgagccaacgtcctgaacaaatgatggctgctctgtatctacaagttagcatcataagccaggctctcatcttcgtcaccagatcccgtagctggtcctacgccgaatgccctggccttctcttacttggtgcatttgtcatagctcagctggtggcgacatttatagcagtttatgcaaactggagttttgcccggatagaaggagccggttggggatgggctggagtgatctggctatacagtttcctaacgtacatccctcttgacttactcaagtttggaatccgctatgttttgagtggaaaagcttggttaaatcttcttgagaacaagactgccttcacgacgaagaaagactatgggaaagaggaaagagaagctcaatgggctgcagctcaaagaaccctccacggacttcaaccagcagagaaaaacaacatctttaacgaaaagaatagttacagtgaactctctcagattgctgaacaggctaaacgccgagctgaggttgtcaggctgagagagataaatacattaaaagggcatgtagagtcagtggtgaagcttaaaggacttgacattgacacgattcagcaacattacaccgtttaaaacccagctttcttgtacaaagttggcattataagaaagcattgcttatcaatttgttgcaacgaacaggtcactatcagtcaaaataaaatcattatttgttaattaactagttctagagcggccgcccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttact

SEQ ID NO: 46 - DNA, 2356 bp, Arabidopsis thaliana
  misc_feature 1..2356: note = pAtAHA5
  source 1..2356: mol_type = genomic DNA
aaccatcatgacaagcgtctggatttttggaatctaggattttttgaacatatagtgtggtgattttttggggagaagagatcattgttgatgtatgttgcctgagttatggccgcgacgaaaaaatagactgaaatgtgtgaaaagatggtataaatatctttacctaggtttctaggttttgattactgggcatgtgaaatgggttttaataaatgggcttgggcttgtaaaattgtggaattaagctattgtaatcacgcatatgagttatctaattggataacttattagattgtaataatgagggaaaaaaaaagattattataatgagaggtccacatttaaaatgatgtgacactcttagcaaataaatataacatggcaaaatttagaagtgttgtcaaaaaacgataagcgtaaaatggaaattgtgactgaaccaccataagaagcgtctggatttttggagtccgagagatttagaaagtggagtgtggttgttttttttagagggggaatgttgttgtttatgccattagagtcatggtggcgtcgaagaacaataagtatatgtgggaatcagagatgtgtgtaagagaaacaaaaatttatttggttgggttttgaattttcaagaaaatgttaaattatttcatcgagtttctttttttttctctatgttaggaaatggtatggtcaaaatctattgatgatggtgatgcctaacattaacaaaaccaatagaagggagacaagagacataagacatcttcttgaaattcatcaacattacaaacttactaaaactcctcctcccaatcccaaatgaatatgtacttcttaaagccacgtgtctttctctttttcattaatatttatttatttttttaaaatgtgaaattatttctttggtttaatatctctggatcattcttctacaatgatcacaagtatttagatcatgtgtaaatagtcccatcatgaagttttaattaccaatcggtattgcacatcaccgattggtactgatccacctgcacttttgtcgatcttttgggacaacgaaaaagaaactaatttaatatattttgttatccgctcgaagatattaattataagctttaatttggaacttctttatatcaattatatatatatataacttctttgttaatagaaaatgggaacgatccaaaacttaagacatttgatctccacaaggccgtcgacgtacagagaagaagaatggccaaaaataagtaaataaaagagaagaaaccgaggggaagatgattttgcattgccacgacccacgagcatccatcggccatcgcctttttggattcacatggcaaagacaaacttcgatcttccttttttctcgtgaagttattatctctctaatttctagtctggtcgaacctatatttcatcatatatatcattatttaattcatatgctgatgcatgggacattatcatgttaagtcattacaaaaaaataaaaataaaaataacttacgttggactctcagtttaccttctcctatggatttcacttccttgtataagaatatacatcttactcaaatcatgatttatatgcaattttcttttgtgagattttttttacatataaaccagaaatgttgaaagaagaatatccaaacacaacaaaataatagaagaaatctcacgtagaacatggaaatggaaaaagaagtgaactcgtgaaccgatatatttaccaaaagtgcacacatagacaagtctttctcattaaagcccaatttcacactttcacattttcgtgacgtcttttgaagttttgaatacgaattaatgttcatgtacgctataaagacctaaaaggttgtcatatttctccactttcatcttctcttcacaactatcctaaaccttttttccttttcttcttcttcactctctttcttcccggcgtcactatggcggctgccgccgggtaaaggcagcgaactcgaccacatcaagaatgaatccgttgatctggtatatatatttacgtcttcttagttttctattatctatatgttttaaaaaatctttggccctactaattttaaattttctgttacataaaagatacatatatgtatagtacgtaactagtaactactttgatctaatcagttaatatatttgtcccatgcgatttactcatgtttccattagaaatgattagtcaaaattatataaatgatgggtctaggtacgaataccctaatatatttgtcccatgcgatttactcatgtttccattagaaatgattagtcaaaattatataaatgatgggtctaggtacgaataccc

SEQ ID NO: 47 - DNA, 6253 bp, synthetic construct
  misc_feature 1..6253: note = Cassette pAtAHA5-AtAHA5W857L
  source 1..6253: mol_type = other DNA
aaccatcatgacaagcgtctggatttttggaatctaggattttttgaacatatagtgtggtgattttttggggagaagagatcattgttgatgtatgttgcctgagttatggccgcgacgaaaaaatagactgaaatgtgtgaaaagatggtataaatatctttacctaggtttctaggttttgattactgggcatgtgaaatgggttttaataaatgggcttgggcttgtaaaattgtggaattaagctattgtaatcacgcatatgagttatctaattggataacttattagattgtaataatgagggaaaaaaaaagattattataatgagaggtccacatttaaaatgatgtgacactcttagcaaataaatataacatggcaaaatttagaagtgttgtcaaaaaacgataagcgtaaaatggaaattgtgactgaaccaccataagaagcgtctggatttttggagtccgagagatttagaaagtggagtgtggttgttttttttagagggggaatgttgttgtttatgccattagagtcatggtggcgtcgaagaacaataagtatatgtgggaatcagagatgtgtgtaagagaaacaaaaatttatttggttgggttttgaattttcaagaaaatgttaaattatttcatcgagtttctttttttttctctatgttaggaaatggtatggtcaaaatctattgatgatggtgatgcctaacattaacaaaaccaatagaagggagacaagagacataagacatcttcttgaaattcatcaacattacaaacttactaaaactcctcctcccaatcccaaatgaatatgtacttcttaaagccacgtgtctttctctttttcattaatatttatttatttttttaaaatgtgaaattatttctttggtttaatatctctggatcattcttctacaatgatcacaagtatttagatcatgtgtaaatagtcccatcatgaagttttaattaccaatcggtattgcacatcaccgattggtactgatccacctgcacttttgtcgatcttttgggacaacgaaaaagaaactaatttaatatattttgttatccgctcgaagatattaattataagctttaatttggaacttctttatatcaattatatatatatataacttctttgttaatagaaaatgggaacgatccaaaacttaagacatttgatctccacaaggccgtcgacgtacagagaagaagaatggccaaaaataagtaaataaaagagaagaaaccgaggggaagatgattttgcattgccacgacccacgagcatccatcggccatcgcctttttggattcacatggcaaagacaaacttcgatcttccttttttctcgtgaagttattatctctctaatttctagtctggtcgaacctatatttcatcatatatatcattatttaattcatatgctgatgcatgggacattatcatgttaagtcattacaaaaaaataaaaataaaaataacttacgttggactctcagtttaccttctcctatggatttcacttccttgtataagaatatacatcttactcaaatcatgatttatatgcaattttcttttgtgagattttttttacatataaaccagaaatgttgaaagaagaatatccaaacacaacaaaataatagaagaaatctcacgtagaacatggaaatggaaaaagaagtgaactcgtgaaccgatatatttaccaaaagtgcacacatagacaagtctttctcattaaagcccaatttcacactttcacattttcgtgacgtcttttgaagttttgaatacgaattaatgttcatgtacgctataaagacctaaaaggttgtcatatttctccactttcatcttctcttcacaactatcctaaaccttttttccttttcttcttcttcactctctttcttcccggcgtcactatggcggctgccgccgggtaaaggcagcgaactcgaccacatcaagaatgaatccgttgatctggtatatatatttacgtcttcttagttttctattatctatatgttttaaaaaatctttggccctactaattttaaattttctgttacataaaagatacatatatgtatagtacgtaactagtaactactttgatctaatcagttaatatatttgtcccatgcgatttactcatgtttccattagaaatgattagtcaaaattatataaatgatgggtctaggtacgaataccctaatatatttgtcccatgcgatttactcatgtttccattagaaatgattagtcaaaattatataaatgatgggtctaggtacgaatacccatgcaagtttgtacaaaaaagcaggcttaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtacccagctttcttgtacaaagtggctatggaggaagtgttcgaggagctgaaatgtacaaaacaaggtcttacggcaaatgaagcttctcaccgtcttgacgtttttggtcctaacaagcttgaagagaagaaggaaagcaagttacttaagtttttagggttcatgtggaacccattgtcgtgggtgatggaggtggctgcactaatggcaatcgctttggccaacggaggaggacggccaccggattggcaagactttgtggggatagtgtgtttactattaatcaactcaaccattagtttcattgaggaaaataacgccggtaatgctgcagccgctctcatggccggtctagcccctaagacaaaggttctcagagataatcaatggagcgagcaagaagcatcgattctcgtccccggagatgtaatcagcatcaagctcggcgatatcattcccgccgatgcacgtttactcgacggcgatcctctcaaaatcgatcaatcatcactcaccggtgaatcaatccccgtcacaaaaaaccccagcgatgaagtcttctccggctcaatatgcaaacaaggcgaaatcgaagcaatcgtcatcgccactggagtccacacattcttcggcaaagcagctcatctcgtcgacaacacaaaccaaatcggccatttccagaaagttctcacttcgattggtaacttctgtatctgttcgattgctttaggaatcatcgttgagcttttagtaatgtatccgattcaacgccggagatatcgagacggaatcgataatttacttgttttgttgatcggagggattcctatagctatgcctagtgtgttatctgttacaatggcgactggttctcatagattgtttcaacaaggagctattactaagagaatgactgctattgaggaaatggctggtatggatgtattgtgttgtgataagactggtactcttacgcttaataagcttacggttgataagaacttagtcgaggttttcgctaaaggagttggtaaagaacatgttttccttttggctgcgagagcttcgaggattgagaatcaagatgcgattgatgctgctattgttggaatgcttgctgatcctaaagaggctagagctggtgttagagaggttcattttttcccttttaatccggttgataagagaactgctttgacttatgttgactctgatggaaactggcatagagctagtaaaggagctccagagcagatactgaacctctgtaattgcaaggaagatgttaggaggaaagttcatggagtgattgataagtttgctgagcgtggacttcgctctttggctgttgcaagacaggaagttctcgagaagaagaaagatgctcctggtggcccatggcaacttgtaggtcttttacctctttttgatcctccgaggcatgacagtgccgagacaatcaggagggctttaaacctcggtgttaatgttaagatgataaccggggaccagcttgcaattgggaaagagaccggtcgaaggcttggaatgggtacgaacatgtatccttcttctgcgctgctcggacaagtcaaagattcttccttgggtgcacttcctgtggatgaactgatagagaaggctgatgggtttgcaggagtctttccagaacataagtatgagattgttcataggctgcaacaaaggaatcacatatgtggcatgactggagatggtgtgaatgatgccccggctctcaagaaggcggatattggtatagctgttgttgatgctactgatgctgcgagaggcgcttctgatatcgttctcacagaacccggattgagtgttatcattagtgctgtactcaccagtagagccatattccaaagaatgaaaaattacactatttatgcggtttcaatcacgatacgaattgtgtttggcttcatgttcattgccctcatatggcagttcgatttttcgcctttcatggttctgatcatagcaatcttaaacgacggaacaatcatgacaatatcaaaagacagaatgaagccatctccacagccagatagctggaaactcagagatatattctcgaccggcgtcgtgcttggaggttaccaggccttgatgacagtcgttttcttctgggtgatgaaagacagtgattttttctcgaactattttggtgtgagaccactgagccaacgtcctgaacaaatgatggctgctctgtatctacaagttagcatcataagccaggctctcatcttcgtcaccagatcccgtagctggtcctacgccgaatgccctggccttctcttacttggtgcatttgtcatagctcagctggtggcgacatttatagcagtttatgcaaactggagttttgcccggatagaaggagccggttggggatgggctggagtgatctggctatacagtttcctaacgtacatccctcttgacttactcaagtttggaatccgctatgttttgagtggaaaagcttggttaaatcttcttgagaacaagactgccttcacgacgaagaaagactatgggaaagaggaaagagaagctcaattggctgcagctcaaagaaccctccacggacttcaaccagcagagaaaaacaacatctttaacgaaaagaatagttacagtgaactctctcagattgctgaacaggctaaacgccgagctgaggttgtcaggctgagagagataaatacattaaaagggcatgtagagtcagtggtgaagcttaaaggacttgacattgacacgattcagcaacattacaccgtttaacaactttattatacatagttgataattcactggccgtcgttttacaacgtcgtgactgggaaaacgatctaactgactagccgcggccatggcggccgggagcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacct

SEQ ID NO: 48 - DNA, 6253 bp, synthetic construct
  misc_feature 1..6253: note = Cassette pAtAHA5-AtAHA5 WT
  source 1..6253: mol_type = other DNA
aaccatcatgacaagcgtctggatttttggaatctaggattttttgaacatatagtgtggtgattttttggggagaagagatcattgttgatgtatgttgcctgagttatggccgcgacgaaaaaatagactgaaatgtgtgaaaagatggtataaatatctttacctaggtttctaggttttgattactgggcatgtgaaatgggttttaataaatgggcttgggcttgtaaaattgtggaattaagctattgtaatcacgcatatgagttatctaattggataacttattagattgtaataatgagggaaaaaaaaagattattataatgagaggtccacatttaaaatgatgtgacactcttagcaaataaatataacatggcaaaatttagaagtgttgtcaaaaaacgataagcgtaaaatggaaattgtgactgaaccaccataagaagcgtctggatttttggagtccgagagatttagaaagtggagtgtggttgttttttttagagggggaatgttgttgtttatgccattagagtcatggtggcgtcgaagaacaataagtatatgtgggaatcagagatgtgtgtaagagaaacaaaaatttatttggttgggttttgaattttcaagaaaatgttaaattatttcatcgagtttctttttttttctctatgttaggaaatggtatggtcaaaatctattgatgatggtgatgcctaacattaacaaaaccaatagaagggagacaagagacataagacatcttcttgaaattcatcaacattacaaacttactaaaactcctcctcccaatcccaaatgaatatgtacttcttaaagccacgtgtctttctctttttcattaatatttatttatttttttaaaatgtgaaattatttctttggtttaatatctctggatcattcttctacaatgatcacaagtatttagatcatgtgtaaatagtcccatcatgaagttttaattaccaatcggtattgcacatcaccgattggtactgatccacctgcacttttgtcgatcttttgggacaacgaaaaagaaactaatttaatatattttgttatccgctcgaagatattaattataagctttaatttggaacttctttatatcaattatatatatatataacttctttgttaatagaaaatgggaacgatccaaaacttaagacatttgatctccacaaggccgtcgacgtacagagaagaagaatggccaaaaataagtaaataaaagagaagaaaccgaggggaagatgattttgcattgccacgacccacgagcatccatcggccatcgcctttttggattcacatggcaaagacaaacttcgatcttccttttttctcgtgaagttattatctctctaatttctagtctggtcgaacctatatttcatcatatatatcattatttaattcatatgctgatgcatgggacattatcatgttaagtcattacaaaaaaataaaaataaaaataacttacgttggactctcagtttaccttctcctatggatttcacttccttgtataagaatatacatcttactcaaatcatgatttatatgcaattttcttttgtgagattttttttacatataaaccagaaatgttgaaagaagaatatccaaacacaacaaaataatagaagaaatctcacgtagaacatggaaatggaaaaagaagtgaactcgtgaaccgatatatttaccaaaagtgcacacatagacaagtctttctcattaaagcccaatttcacactttcacattttcgtgacgtcttttgaagttttgaatacgaattaatgttcatgtacgctataaagacctaaaaggttgtcatatttctccactttcatcttctcttcacaactatcctaaaccttttttccttttcttcttcttcactctctttcttcccggcgtcactatggcggctgccgccgggtaaaggcagcgaactcgaccacatcaagaatgaatccgttgatctggtatatatatttacgtcttcttagttttctattatctatatgttttaaaaaatctttggccctactaattttaaattttctgttacataaaagatacatatatgtatagtacgtaactagtaactactttgatctaatcagttaatatatttgtcccatgcgatttactcatgtttccattagaaatgattagtcaaaattatataaatgatgggtctaggtacgaataccctaatatatttgtcccatgcgatttactcatgtttccattagaaatgattagtcaaaattatataaatgatgggtctaggtacgaatacccatgcaagtttgtacaaaaaagcaggcttaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtacccagctttcttgtacaaagtggctatggaggaagtgttcgaggagctgaaatgtacaaaacaaggtcttacggcaaatgaagcttctcaccgtcttgacgtttttggtcctaacaagcttgaagagaagaaggaaagcaagttacttaagtttttagggttcatgtggaacccattgtcgtgggtgatggaggtggctgcactaatggcaatcgctttggccaacggaggaggacggccaccggattggcaagactttgtggggatagtgtgtttactattaatcaactcaaccattagtttcattgaggaaaataacgccggtaatgctgcagccgctctcatggccggtctagcccctaagacaaaggttctcagagataatcaatggagcgagcaagaagcatcgattctcgtccccggagatgtaatcagcatcaagctcggcgatatcattcccgccgatgcacgtttactcgacggcgatcctctcaaaatcgatcaatcatcactcaccggtgaatcaatccccgtcacaaaaaaccccagcgatgaagtcttctccggctcaatatgcaaacaaggcgaaatcgaagcaatcgtcatcgccactggagtccacacattcttcggcaaagcagctcatctcgtcgacaacacaaaccaaatcggccatttccagaaagttctcacttcgattggtaacttctgtatctgttcgattgctttaggaatcatcgttgagcttttagtaatgtatccgattcaacgccggagatatcgagacggaatcgataatttacttgttttgttgatcggagggattcctatagctatgcctagtgtgttatctgttacaatggcgactggttctcatagattgtttcaacaaggagctattactaagagaatgactgctattgaggaaatggctggtatggatgtattgtgttgtgataagactggtactcttacgcttaataagcttacggttgataagaacttagtcgaggttttcgctaaaggagttggtaaagaacatgttttccttttggctgcgagagcttcgaggattgagaatcaagatgcgattgatgctgctattgttggaatgcttgctgatcctaaagaggctagagctggtgttagagaggttcattttttcccttttaatccggttgataagagaactgctttgacttatgttgactctgatggaaactggcatagagctagtaaaggagctccagagcagatactgaacctctgtaattgcaaggaagatgttaggaggaaagttcatggagtgattgataagtttgctgagcgtggacttcgctctttggctgttgcaagacaggaagttctcgagaagaagaaagatgctcctggtggcccatggcaacttgtaggtcttttacctctttttgatcctccgaggcatgacagtgccgagacaatcaggagggctttaaacctcggtgttaatgttaagatgataaccggggaccagcttgcaattgggaaagagaccggtcgaaggcttggaatgggtacgaacatgtatccttcttctgcgctgctcggacaagtcaaagattcttccttgggtgcacttcctgtggatgaactgatagagaaggctgatgggtttgcaggagtctttccagaacataagtatgagattgttcataggctgcaacaaaggaatcacatatgtggcatgactggagatggtgtgaatgatgccccggctctcaagaaggcggatattggtatagctgttgttgatgctactgatgctgcgagaggcgcttctgatatcgttctcacagaacccggattgagtgttatcattagtgctgtactcaccagtagagccatattccaaagaatgaaaaattacactatttatgcggtttcaatcacgatacgaattgtgtttggcttcatgttcattgccctcatatggcagttcgatttttcgcctttcatggttctgatcatagcaatcttaaacgacggaacaatcatgacaatatcaaaagacagaatgaagccatctccacagccagatagctggaaactcagagatatattctcgaccggcgtcgtgcttggaggttaccaggccttgatgacagtcgttttcttctgggtgatgaaagacagtgattttttctcgaactattttggtgtgagaccactgagccaacgtcctgaacaaatgatggctgctctgtatctacaagttagcatcataagccaggctctcatcttcgtcaccagatcccgtagctggtcctacgccgaatgccctggccttctcttacttggtgcatttgtcatagctcagctggtggcgacatttatagcagtttatgcaaactggagttttgcccggatagaaggagccggttggggatgggctggagtgatctggctatacagtttcctaacgtacatccctcttgacttactcaagtttggaatccgctatgttttgagtggaaaagcttggttaaatcttcttgagaacaagactgccttcacgacgaagaaagactatgggaaagaggaaagagaagctcaatgggctgcagctcaaagaaccctccacggacttcaaccagcagagaaaaacaacatctttaacgaaaagaatagttacagtgaactctctcagattgctgaacaggctaaacgccgagctgaggttgtcaggctgagagagataaatacattaaaagggcatgtagagtcagtggtgaagcttaaaggacttgacattgacacgattcagcaacattacaccgtttaacaactttattatacatagttgataattcactggccgtcgttttacaacgtcgtgactgggaaaacgatctaactgactagccgcggccatggcggccgggagcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacct

SEQ ID NO: 49 - DNA, 2886 bp, Zea mays
  misc_feature 1..2886: note = ZmAHA5W885L version 1
  source 1..2886: mol_type = other DNA
atggggccactgcagcggcggcctacggctatgggggggctggaggagattaagaacgaggcggttgacctggagaacatcccgattgaggaggtgttcgagcagctgaagtgcacccgcgagggcctctccagctcggaggggcagcagcgcctggagatcttcggccccaaccgcctggaggagaagaaggagtccaagatcctcaagttcctgggcttcatgtggaacccactgagctgggttatggagatggctgccgtgatggctatcgcgctcgctaatggcgggggcaagccaccagactgggaggatttcgtcggcatcattgtgctcctggtcatcaactcgacgatttctttcatcgaggagaacaatgctggcaacgctgccgcggctctgatggctaatctcgctcctaagacaaaggtgctgagggatggccgctggggggagcaggaggccgcgatcctggtgccgggcgacattgtcagcatcaagctcggggacatcgttccagctgatgctaggctcctggagggggacccactcaaggtggatcagagcgctctgacgggcgagtcgctccctgtcacaaaggggccaggcgacgaggttttctccggcagcacgtgcaagcagggggagattgaggccgtggtcatcgcgactggcgtccacaccttcttcgggaaggctgcccatctggtcgactcgactaaccaggttggccacttccagcaggtgctcaccgcgatcgggaatttctgcatttgctctatcgctgtgggcattgttgtggagatcattgtcatgttcccaatccagcataggcgctaccgctccggcattgagaacctcctggtgctcctgatcgggggcatccccattgcgatgccaacggttctgagcgtgacaatggccatcggcagccacaagctctcgcagcagggggccattacgaagaggatgacagccatcgaggagatggcggggatggacgtcctgtgctctgataagacaggcactctcaccctgaacaagctgtcagtggacaagaatctcgttgaggtgttctgcaagggcgttgacaaggatcacgtgctcctgctcgcggctcgggcctcccgcaccgagaaccaggacgcgatcgatgctgcgatggtggggatgctcgccgatccaaaggaggctagggccggcattcgggagatccatttcctgcccttcaatccagtcgacaagaggactgctctcacctacatcgacgccgatggccactggcatcgggtgagcaagggggccccagagcagatcctcgacctgtgccactgcaaggaggatctgcggcgcaaggtgcatggcatcattgacaagtacgcggagagggggctcaggtcgctggcggtcgctcggcaggaggttcccgagcgcaacaaggagtctcctgggggcccgtggcagttcgtcggcctgctcccactcttcgacccacctcggcacgattccgcggagactatccgcaaggctctcgtgctgggcgtcaatgttaagatgattaccggggatcagctggccatcggcaaggagacggggaggaggctcgggatgggcacaaacatgtacccgtcttcagcgctgctcggccagaataaggacgctaccctggaggccctcccggtggacgagctgatcgagaaggcggatggcttcgctggggtcttcccggagcacaagtacgagattgttaagaggctgcaggagaagaagcatatcgtgggcatgacgggggacggcgtcaacgatgctcctgctctcaagaaggcggatatcggcattgctgtcgctgacgctacggatgctgctcggtcggcctctgacatcgttctgacagagccgggcctctctgtgatcatttcagcggtcctgacctcacgctgcattttccagaggatgaagaactacactatctacgcggtttccatcaccattcgcatcgtgctcggcttcatgctcattgccctgatctggcagtacgacttctctccgttcatggtcctgatcattgccatcctcaatgacggcacgattatgacaatctcaaaggatagggtgaagccatcgcctctgccggactcttggaagctcaaggagattttcgccacaggcatcgtgctggggtcctacctcgctctgatgactgtcatcttcttctgggccatgcacaagaccgacttcttcagcgataagttcggcgtccgctccatcagggacagcgagcatgagatgatgtcggcgctctacctgcaggtctcaattgtttcccaggccctcatcttcgtgacccggtcacgctcctggagcttcgttgagaggccaggcctgctcctggtgacggccttcctcctggcgcagctggtcgctacattcctcgccgtttacgcgaactggggcttcgcgcgcattaaggggatcggctgggggtgggctggcgtcgtttggctgtactccatcgtcttctacttcccgctcgacctgattaagttcttcatcaggttcgtgctcagcggccgggcgtgggataacctcctggagaataagacggccttcaccacgaagaaggactacggcagggaggagcgcgaggctcagctcgctactgctcagcgcaccctccacggcctgcagccaccagaggctgctacgtcgacactgttccatgacaagaactcatacagggagctctccgagattgctgagcaggccaagcgcagggcggagatcgctaggctcagggagctgaacaccctcaaggggcacgtcgagtccgtggtcaagctgaagggcctcgacattgatactatccagcagaattacaccgtgtag

SEQ ID NO: 50 - DNA, 2886 bp, Zea mays
  misc_feature 1..2886: note = ZmAHA5W885L version 2
  source 1..2886: mol_type = other DNA
atgggcccactgcagaggcgcccaacagcgatggggggcctggaggagatcaagaacgaggccgtcgatctggagaacatccctattgaggaggtgttcgagcagctgaagtgcacccgggagggcctcagctcctcggagggccagcagcgcctggagatcttcggcccaaaccggctggaggagaagaaggagagcaagatcctcaagttcctggggttcatgtggaacccgctgtcctgggtcatggagatggctgccgtgatggcgattgctctggctaatggcgggggcaagccaccggactgggaggatttcgtggggattatcgtcctgctcgtcatcaacagcactatctccttcattgaggagaacaacgctggcaacgccgctgctgcgctgatggcgaacctcgctccaaagactaaggtcctccgcgacggccgctggggggagcaggaggctgcgattctcgtgcccggggacatcgttagcattaagctcggggatatcgtccctgctgatgcccggctgctggagggggaccccctgaaggtcgaccagagcgctctcaccggcgagtcgctcccagtcactaagggccctggggacgaggtgttcagcggctctacgtgcaagcagggcgagatcgaggcggtcgtgatcgctaccggcgtgcacaccttcttcgggaaggccgcccatctcgtggattcgacaaaccaggtcgggcacttccagcaggtgctgacagccatcgggaacttctgcatctgctcgattgccgttgggatcgtggttgagattattgtgatgttcccgattcagcaccggcgctaccgctccgggatcgagaacctcctggtcctcctcattggggggattccgatcgctatgccgaccgtcctgagcgtcactatggctatcgggtctcacaagctctcacagcagggggcgattaccaagcggatgacagcgattgaggagatggcggggatggacgtgctgtgctcagacaagaccggcactctgacgctcaacaagctctccgtggacaagaacctcgtcgaggtgttctgcaagggggtggacaaggatcatgtcctgctgctggcggccagggcctcccggacagagaaccaggacgccattgacgccgctatggtggggatgctggccgaccctaaggaggcgagggccggcatccgcgagattcacttcctgccattcaatcccgttgacaagcgcactgcgctgacgtacattgatgcggacgggcactggcacagggtgtccaagggggctccagagcagatcctcgatctgtgccactgcaaggaggatctgcggcgcaaggtgcatgggatcattgacaagtacgcggagagggggctccggagcctggcggtggccaggcaggaggtgccggagcgcaacaaggagtcgccggggggcccttggcagttcgtcgggctgctccctctgttcgatcccccgaggcacgacagcgcggagactatccgcaaggccctcgttctgggggtgaatgtcaagatgattaccggggaccagctggccattgggaaggagacaggccggcgcctcgggatgggcacgaacatgtacccgtcatccgccctgctggggcagaataaggacgccactctggaggccctgccggtcgatgagctcatcgagaaggctgacgggttcgcgggcgtcttccccgagcataagtacgagatcgtgaagcggctgcaggagaagaagcatattgtggggatgactggcgacggggtgaatgatgctcccgccctgaagaaggctgacatcggcattgccgtcgcggacgccacagatgctgcccgctcggcgtccgatatcgttctcacggagcctgggctgagcgttattatctcggcggttctgacaagcaggtgcatcttccagcgcatgaagaactacaccatctacgctgtgtcgatcactatcaggatcgtgctgggcttcatgctgattgcgctcatctggcagtacgacttctcccctttcatggtcctgatcattgctatcctcaacgacggcacaatcatgactatctcgaaggaccgcgtgaagccgagcccactccccgactcttggaagctcaaggagattttcgccactgggatcgttctcgggtcttacctcgctctgatgaccgtgattttcttctgggctatgcacaagaccgacttcttcagcgacaagttcggggttcgctcgattcgcgactcggagcacgagatgatgtctgcgctgtacctccaggtttcaattgtttcacaggcgctcattttcgtgactaggtcacggtcttggtccttcgtcgagaggcctggcctgctcctggtcactgctttcctgctcgcccagctggtggccactttcctcgctgtttacgctaattgggggttcgctaggatcaaggggatcggctgggggtgggcgggcgtggtctggctctactccatcgttttctacttcccactcgacctcatcaagttcttcattcggttcgtgctgtcagggagggcgtgggacaacctcctggagaacaagaccgcgttcaccactaagaaggactacggccgcgaggagagggaggcgcagctggctacagcgcagcgcaccctccacgggctccagcccccagaggctgcgacatctacactcttccacgacaagaattcttaccgcgagctgtcagagattgccgagcaggctaagcgcagggcggagattgctaggctgagggagctcaacacactgaagggccacgtggagtcagtggtcaagctgaagggcctggacatcgatacaattcagcagaactacaccgtttag

SEQ ID NO: 51 - DNA, 504 bp, Cassava vein mosaic virus
  misc_feature 1..504: note = proCsVMV
  source 1..504: mol_type = genomic DNA
aaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgt

SEQ ID NO: 52 - DNA, 557 bp, synthetic construct
  misc_feature 1..557: note = Rice actin intron
  source 1..557: mol_type = other DNA
accaccaccaccaccaccacctccacctcctcccccctcgctgccggacgacgagctcctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtaga

SEQ ID NO: 53 - DNA, 509 bp, Arabidopsis thaliana
  misc_feature 1..509: note = terSac66
  source 1..509: mol_type = genomic DNA
ctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 54 - DNA, 968 bp, Zea mays
  misc_feature 1..968: note = proZmrbcS_Mut_al
  source 1..968: mol_type = other DNA
tccctttaatctggcgctagatctgcatccgcggcttgcaaagataaatggcacatttagtgtgttattttgcaatacctttcatagtagatatccttaaatgcagttttaggcatgtttgggtaattaaataacatttttaggaggagttttagatttacctttctttcgtgatgactgatgacagacgtggggaattcaaatgcaactctagcgaaagttcatatatttttcataaatagctgaggctggggtaattattttttttgtagaaaaatagaataggtggaatggttggggaaggcgtaggcgctcgtggacgacgcccgataaaagacaagaggcggaattgccatgaattcgaggtagctaagtaaggcgcatatatatgccaaaaaattctactgtcactttccaatttcaatgcgctgccaaacaagccatcctggaaactgacttgaattcagcccaattctgtagatccaaacagggccggcgtcagtgcctcaggtgagagagcagcagacgattcaaagagccaaaactgcaagcagacgcagccgaagccgaagccgaagcccaagcccaaaactgttttgtctttgcccagaaccgcgacgagcctaaactgcgcttcctcctatctacaagtccctggcacatcacgcatagtccaacctaggcgcgcaggcgataaggcgcgccacggggacgcgacatgtggtggcggacgcgatcaggatagggccaggctggccgggcgcggccacgggagaacggtggccactcgtcccacatccgcttcgtcctgtcctgtactgcgtcctgcccccaacgagagccggagccggccatcccgtcgcacactctccccctctatatatgccgtcggtgtgggggagcctactacaggacgacccaagcaagcaagcaagcagcgagtacatacatactaggcagccaggcag

SEQ ID NO: 55 - DNA, 537 bp, Zea mays
  misc_feature 1..537: note = terZmRbcs
  source 1..537: mol_type = genomic DNA
tagaccgcgcccgccggccgccccccgccggctagctagctagctagctagctcctgcgtgagctagtagctagtgccatgcgtcgtctctgtcgttcggttttgcttcgggtcaccgtaccctttgcttgcttggtttcttctttccttttttccttttttttttcttcttttccccggccatggttcctttgctttcagcagttctctgctggatgtgatgtatccattgttgcaagcatggccttgcattggctacctctatacctgctacaaactactgcaacgcctatatatacttggggtgaggaacatgtgaatgcaagctccggctatcatatacatgtaatatggatacaaactatatatataaatccgccgaggcgccgactaatactatacgacgacaccgtgttaagttaatatataactggtgctttttatttatatatctgtctcatcatatatatatgctaattaatggatgtgtgtcctcttcacttcaattccttctttcctttcctatgctttgagatc

SEQ ID NO: 56 - DNA, 444 bp, Zea mays
  misc_feature 1..444: note = proZmRAB17_del
  source 1..444: mol_type = genomic DNA
aaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcatc

SEQ ID NO: 57 - DNA, 500 bp, Zea mays
  misc_feature 1..500: note = terZmRab17
  source 1..500: mol_type = genomic DNA
gcggcgcctatacgtggctgtgctgtgctgtctggcgcgtcaaagccgtactcttcagcgttccatagataataagataaacccatgaataagtgtccctaccctttgatcatgtgacagggacagggacagtttcctgtacttgtggggtgaatttgtacgtgtgatacggtaacttggtccgtgtactgtttatgctgctacgtttccttcctaaaagtacgtatatatatacagcacttgcgagtggctttacttgtgtgcactgcctaaaagtgtcgatattacctccagcggtaaataaatgacccttgtaagaaacttacacagcatgtcttactaattaacagccgttattatgttcggcgcagtactcacactggaacttacgaacaagaattgctctatcaatcaccatctactttagatatatcttctgaaacttttttcaaatccaaaagttcagtatcttcctacaaattagtgaattaggaatccaa

SEQ ID NO: 58 - DNA, 4416 bp, synthetic construct
  misc_feature 1..4416: note = Cassette_T02781_CsVMV ZmAHA5_W885L
  source 1..4416: mol_type = other DNA
aaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtccctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatggggccactgcagcggcggcctacggctatgggggggctggaggagattaagaacgaggcggttgacctggagaacatcccgattgaggaggtgttcgagcagctgaagtgcacccgcgagggcctctccagctcggaggggcagcagcgcctggagatcttcggccccaaccgcctggaggagaagaaggagtccaagatcctcaagttcctgggcttcatgtggaacccactgagctgggttatggagatggctgccgtgatggctatcgcgctcgctaatggcgggggcaagccaccagactgggaggatttcgtcggcatcattgtgctcctggtcatcaactcgacgatttctttcatcgaggagaacaatgctggcaacgctgccgcggctctgatggctaatctcgctcctaagacaaaggtgctgagggatggccgctggggggagcaggaggccgcgatcctggtgccgggcgacattgtcagcatcaagctcggggacatcgttccagctgatgctaggctcctggagggggacccactcaaggtggatcagagcgctctgacgggcgagtcgctccctgtcacaaaggggccaggcgacgaggttttctccggcagcacgtgcaagcagggggagattgaggccgtggtcatcgcgactggcgtccacaccttcttcgggaaggctgcccatctggtcgactcgactaaccaggttggccacttccagcaggtgctcaccgcgatcgggaatttctgcatttgctctatcgctgtgggcattgttgtggagatcattgtcatgttcccaatccagcataggcgctaccgctccggcattgagaacctcctggtgctcctgatcgggggcatccccattgcgatgccaacggttctgagcgtgacaatggccatcggcagccacaagctctcgcagcagggggccattacgaagaggatgacagccatcgaggagatggcggggatggacgtcctgtgctctgataagacaggcactctcaccctgaacaagctgtcagtggacaagaatctcgttgaggtgttctgcaagggcgttgacaaggatcacgtgctcctgctcgcggctcgggcctcccgcaccgagaaccaggacgcgatcgatgctgcgatggtggggatgctcgccgatccaaaggaggctagggccggcattcgggagatccatttcctgcccttcaatccagtcgacaagaggactgctctcacctacatcgacgccgatggccactggcatcgggtgagcaagggggccccagagcagatcctcgacctgtgccactgcaaggaggatctgcggcgcaaggtgcatggcatcattgacaagtacgcggagagggggctcaggtcgctggcggtcgctcggcaggaggttcccgagcgcaacaaggagtctcctgggggcccgtggcagttcgtcggcctgctcccactcttcgacccacctcggcacgattccgcggagactatccgcaaggctctcgtgctgggcgtcaatgttaagatgattaccggggatcagctggccatcggcaaggagacggggaggaggctcgggatgggcacaaacatgtacccgtcttcagcgctgctcggccagaataaggacgctaccctggaggccctcccggtggacgagctgatcgagaaggcggatggcttcgctggggtcttcccggagcacaagtacgagattgttaagaggctgcaggagaagaagcatatcgtgggcatgacgggggacggcgtcaacgatgctcctgctctcaagaaggcggatatcggcattgctgtcgctgacgctacggatgctgctcggtcggcctctgacatcgttctgacagagccgggcctctctgtgatcatttcagcggtcctgacctcacgctgcattttccagaggatgaagaactacactatctacgcggtttccatcaccattcgcatcgtgctcggcttcatgctcattgccctgatctggcagtacgacttctctccgttcatggtcctgatcattgccatcctcaatgacggcacgattatgacaatctcaaaggatagggtgaagccatcgcctctgccggactcttggaagctcaaggagattttcgccacaggcatcgtgctggggtcctacctcgctctgatgactgtcatcttcttctgggccatgcacaagaccgacttcttcagcgataagttcggcgtccgctccatcagggacagcgagcatgagatgatgtcggcgctctacctgcaggtctcaattgtttcccaggccctcatcttcgtgacccggtcacgctcctggagcttcgttgagaggccaggcctgctcctggtgacggccttcctcctggcgcagctggtcgctacattcctcgccgtttacgcgaactggggcttcgcgcgcattaaggggatcggctgggggtgggctggcgtcgtttggctgtactccatcgtcttctacttcccgctcgacctgattaagttcttcatcaggttcgtgctcagcggccgggcgtgggataacctcctggagaataagacggccttcaccacgaagaaggactacggcagggaggagcgcgaggctcagctcgctactgctcagcgcaccctccacggcctgcagccaccagaggctgctacgtcgacactgttccatgacaagaactcatacagggagctctccgagattgctgagcaggccaagcgcagggcggagatcgctaggctcagggagctgaacaccctcaaggggcacgtcgagtccgtggtcaagctgaagggcctcgacattgatactatccagcagaattacaccgtgtagaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 59 - DNA, 4402 bp, synthetic construct
  misc_feature 1..4402: note = Cassette_T02784_rbcS_Mut_al ZmAHA5_W885L
  source 1..4402: mol_type = other DNA
tccctttaatctggcgctagatctgcatccgcggcttgcaaagataaatggcacatttagtgtgttattttgcaatacctttcatagtagatatccttaaatgcagttttaggcatgtttgggtaattaaataacatttttaggaggagttttagatttacctttctttcgtgatgactgatgacagacgtggggaattcaaatgcaactctagcgaaagttcatatatttttcataaatagctgaggctggggtaattattttttttgtagaaaaatagaataggtggaatggttggggaaggcgtaggcgctcgtggacgacgcccgataaaagacaagaggcggaattgccatgaattcgaggtagctaagtaaggcgcatatatatgccaaaaaattctactgtcactttccaatttcaatgcgctgccaaacaagccatcctggaaactgacttgaattcagcccaattctgtagatccaaacagggccggcgtcagtgcctcaggtgagagagcagcagacgattcaaagagccaaaactgcaagcagacgcagccgaagccgaagccgaagcccaagcccaaaactgttttgtctttgcccagaaccgcgacgagcctaaactgcgcttcctcctatctacaagtccctggcacatcacgcatagtccaacctaggcgcgcaggcgataaggcgcgccacggggacgcgacatgtggtggcggacgcgatcaggatagggccaggctggccgggcgcggccacgggagaacggtggccactcgtcccacatccgcttcgtcctgtcctgtactgcgtcctgcccccaacgagagccggagccggccatcccgtcgcacactctccccctctatatatgccgtcggtgtgggggagcctactacaggacgacccaagcaagcaagcaagcagcgagtacatacatactaggcagccaggcagtctccaccatggggccactgcagcggcggcctacggctatgggggggctggaggagattaagaacgaggcggttgacctggagaacatcccgattgaggaggtgttcgagcagctgaagtgcacccgcgagggcctctccagctcggaggggcagcagcgcctggagatcttcggccccaaccgcctggaggagaagaaggagtccaagatcctcaagttcctgggcttcatgtggaacccactgagctgggttatggagatggctgccgtgatggctatcgcgctcgctaatggcgggggcaagccaccagactgggaggatttcgtcggcatcattgtgctcctggtcatcaactcgacgatttctttcatcgaggagaacaatgctggcaacgctgccgcggctctgatggctaatctcgctcctaagacaaaggtgctgagggatggccgctggggggagcaggaggccgcgatcctggtgccgggcgacattgtcagcatcaagctcggggacatcgttccagctgatgctaggctcctggagggggacccactcaaggtggatcagagcgctctgacgggcgagtcgctccctgtcacaaaggggccaggcgacgaggttttctccggcagcacgtgcaagcagggggagattgaggccgtggtcatcgcgactggcgtccacaccttcttcgggaaggctgcccatctggtcgactcgactaaccaggttggccacttccagcaggtgctcaccgcgatcgggaatttctgcatttgctctatcgctgtgggcattgttgtggagatcattgtcatgttcccaatccagcataggcgctaccgctccggcattgagaacctcctggtgctcctgatcgggggcatccccattgcgatgccaacggttctgagcgtgacaatggccatcggcagccacaagctctcgcagcagggggccattacgaagaggatgacagccatcgaggagatggcggggatggacgtcctgtgctctgataagacaggcactctcaccctgaacaagctgtcagtggacaagaatctcgttgaggtgttctgcaagggcgttgacaaggatcacgtgctcctgctcgcggctcgggcctcccgcaccgagaaccaggacgcgatcgatgctgcgatggtggggatgctcgccgatccaaaggaggctagggccggcattcgggagatccatttcctgcccttcaatccagtcgacaagaggactgctctcacctacatcgacgccgatggccactggcatcgggtgagcaagggggccccagagcagatcctcgacctgtgccactgcaaggaggatctgcggcgcaaggtgcatggcatcattgacaagtacgcggagagggggctcaggtcgctggcggtcgctcggcaggaggttcccgagcgcaacaaggagtctcctgggggcccgtggcagttcgtcggcctgctcccactcttcgacccacctcggcacgattccgcggagactatccgcaaggctctcgtgctgggcgtcaatgttaagatgattaccggggatcagctggccatcggcaaggagacggggaggaggctcgggatgggcacaaacatgtacccgtcttcagcgctgctcggccagaataaggacgctaccctggaggccctcccggtggacgagctgatcgagaaggcggatggcttcgctggggtcttcccggagcacaagtacgagattgttaagaggctgcaggagaagaagcatatcgtgggcatgacgggggacggcgtcaacgatgctcctgctctcaagaaggcggatatcggcattgctgtcgctgacgctacggatgctgctcggtcggcctctgacatcgttctgacagagccgggcctctctgtgatcatttcagcggtcctgacctcacgctgcattttccagaggatgaagaactacactatctacgcggtttccatcaccattcgcatcgtgctcggcttcatgctcattgccctgatctggcagtacgacttctctccgttcatggtcctgatcattgccatcctcaatgacggcacgattatgacaatctcaaaggatagggtgaagccatcgcctctgccggactcttggaagctcaaggagattttcgccacaggcatcgtgctggggtcctacctcgctctgatgactgtcatcttcttctgggccatgcacaagaccgacttcttcagcgataagttcggcgtccgctccatcagggacagcgagcatgagatgatgtcggcgctctacctgcaggtctcaattgtttcccaggccctcatcttcgtgacccggtcacgctcctggagcttcgttgagaggccaggcctgctcctggtgacggccttcctcctggcgcagctggtcgctacattcctcgccgtttacgcgaactggggcttcgcgcgcattaaggggatcggctgggggtgggctggcgtcgtttggctgtactccatcgtcttctacttcccgctcgacctgattaagttcttcatcaggttcgtgctcagcggccgggcgtgggataacctcctggagaataagacggccttcaccacgaagaaggactacggcagggaggagcgcgaggctcagctcgctactgctcagcgcaccctccacggcctgcagccaccagaggctgctacgtcgacactgttccatgacaagaactcatacagggagctctccgagattgctgagcaggccaagcgcagggcggagatcgctaggctcagggagctgaacaccctcaaggggcacgtcgagtccgtggtcaagctgaagggcctcgacattgatactatccagcagaattacaccgtgtagaactagaccgcgcccgccggccgccccccgccggctagctagctagctagctagctcctgcgtgagctagtagctagtgccatgcgtcgtctctgtcgttcggttttgcttcgggtcaccgtaccctttgcttgcttggtttcttctttccttttttccttttttttttcttcttttccccggccatggttcctttgctttcagcagttctctgctggatgtgatgtatccattgttgcaagcatggccttgcattggctacctctatacctgctacaaactactgcaacgcctatatatacttggggtgaggaacatgtgaatgcaagctccggctatcatatacatgtaatatggatacaaactatatatataaatccgccgaggcgccgactaatactatacgacgacaccgtgttaagttaatatataactggtgctttttatttatatatctgtctcatcatatatatatgctaattaatggatgtgtgtcctcttcacttcaattccttctttcctttcctatgctttgagatc

SEQ ID NO: 60 - DNA, 3841 bp, synthetic construct
  misc_feature 1..3841: note = Cassette_T10401_RAB17_del ZmAHA5_W885L
  source 1..3841: mol_type = other DNA
aaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcatctctccaccatgggcccactgcagaggcgcccaacagcgatggggggcctggaggagatcaagaacgaggccgtcgatctggagaacatccctattgaggaggtgttcgagcagctgaagtgcacccgggagggcctcagctcctcggagggccagcagcgcctggagatcttcggcccaaaccggctggaggagaagaaggagagcaagatcctcaagttcctggggttcatgtggaacccgctgtcctgggtcatggagatggctgccgtgatggcgattgctctggctaatggcgggggcaagccaccggactgggaggatttcgtggggattatcgtcctgctcgtcatcaacagcactatctccttcattgaggagaacaacgctggcaacgccgctgctgcgctgatggcgaacctcgctccaaagactaaggtcctccgcgacggccgctggggggagcaggaggctgcgattctcgtgcccggggacatcgttagcattaagctcggggatatcgtccctgctgatgcccggctgctggagggggaccccctgaaggtcgaccagagcgctctcaccggcgagtcgctcccagtcactaagggccctggggacgaggtgttcagcggctctacgtgcaagcagggcgagatcgaggcggtcgtgatcgctaccggcgtgcacaccttcttcgggaaggccgcccatctcgtggattcgacaaaccaggtcgggcacttccagcaggtgctgacagccatcgggaacttctgcatctgctcgattgccgttgggatcgtggttgagattattgtgatgttcccgattcagcaccggcgctaccgctccgggatcgagaacctcctggtcctcctcattggggggattccgatcgctatgccgaccgtcctgagcgtcactatggctatcgggtctcacaagctctcacagcagggggcgattaccaagcggatgacagcgattgaggagatggcggggatggacgtgctgtgctcagacaagaccggcactctgacgctcaacaagctctccgtggacaagaacctcgtcgaggtgttctgcaagggggtggacaaggatcatgtcctgctgctggcggccagggcctcccggacagagaaccaggacgccattgacgccgctatggtggggatgctggccgaccctaaggaggcgagggccggcatccgcgagattcacttcctgccattcaatcccgttgacaagcgcactgcgctgacgtacattgatgcggacgggcactggcacagggtgtccaagggggctccagagcagatcctcgatctgtgccactgcaaggaggatctgcggcgcaaggtgcatgggatcattgacaagtacgcggagagggggctccggagcctggcggtggccaggcaggaggtgccggagcgcaacaaggagtcgccggggggcccttggcagttcgtcgggctgctccctctgttcgatcccccgaggcacgacagcgcggagactatccgcaaggccctcgttctgggggtgaatgtcaagatgattaccggggaccagctggccattgggaaggagacaggccggcgcctcgggatgggcacgaacatgtacccgtcatccgccctgctggggcagaataaggacgccactctggaggccctgccggtcgatgagctcatcgagaaggctgacgggttcgcgggcgtcttccccgagcataagtacgagatcgtgaagcggctgcaggagaagaagcatattgtggggatgactggcgacggggtgaatgatgctcccgccctgaagaaggctgacatcggcattgccgtcgcggacgccacagatgctgcccgctcggcgtccgatatcgttctcacggagcctgggctgagcgttattatctcggcggttctgacaagcaggtgcatcttccagcgcatgaagaactacaccatctacgctgtgtcgatcactatcaggatcgtgctgggcttcatgctgattgcgctcatctggcagtacgacttctcccctttcatggtcctgatcattgctatcctcaacgacggcacaatcatgactatctcgaaggaccgcgtgaagccgagcccactccccgactcttggaagctcaaggagattttcgccactgggatcgttctcgggtcttacctcgctctgatgaccgtgattttcttctgggctatgcacaagaccgacttcttcagcgacaagttcggggttcgctcgattcgcgactcggagcacgagatgatgtctgcgctgtacctccaggtttcaattgtttcacaggcgctcattttcgtgactaggtcacggtcttggtccttcgtcgagaggcctggcctgctcctggtcactgctttcctgctcgcccagctggtggccactttcctcgctgtttacgctaattgggggttcgctaggatcaaggggatcggctgggggtgggcgggcgtggtctggctctactccatcgttttctacttcccactcgacctcatcaagttcttcattcggttcgtgctgtcagggagggcgtgggacaacctcctggagaacaagaccgcgttcaccactaagaaggactacggccgcgaggagagggaggcgcagctggctacagcgcagcgcaccctccacgggctccagcccccagaggctgcgacatctacactcttccacgacaagaattcttaccgcgagctgtcagagattgccgagcaggctaagcgcagggcggagattgctaggctgagggagctcaacacactgaagggccacgtggagtcagtggtcaagctgaagggcctggacatcgatacaattcagcagaactacaccgtttagaacgcggcgcctatacgtggctgtgctgtgctgtctggcgcgtcaaagccgtactcttcagcgttccatagataataagataaacccatgaataagtgtccctaccctttgatcatgtgacagggacagggacagtttcctgtacttgtggggtgaatttgtacgtgtgatacggtaacttggtccgtgtactgtttatgctgctacgtttccttcctaaaagtacgtatatatatacagcacttgcgagtggctttacttgtgtgcactgcctaaaagtgtcgatattacctccagcggtaaataaatgacccttgtaagaaacttacacagcatgtcttactaattaacagccgttattatgttcggcgcagtactcacactggaacttacgaacaagaattgctctatcaatcaccatctactttagatatatcttctgaaacttttttcaaatccaaaagttcagtatcttcctacaaattagtgaattaggaatccaa

SEQ ID NO: 61 - DNA, 4328 bp, synthetic construct
  misc_feature 1..4328: note = Cassette_T10584_MYB60 ZmAHA5_W885L
  source 1..4328: mol_type = other DNA
ctccgtcgctagcgttgtaatttttatcagcctctctccatctgattggatgagctgcttctcctagctagcacgttgtcaaccattggggacgccctttaggagtgaaagtgatgcagttactctactcccatccggtccaaatcccaacctaagggatatatgacgaagagttttcaatttcagtataatgtgtatagcaagtgatttgattttcctgcagattcttgagagagaagggccaaacctgcatatgtgccaggtgcgcactcacagtcacacacacggtcctcatcaatgcccaagaaatctggagagacacagacaaacgcaagcaattactccattcatgagcgccacagcagaaaaaggagtcctccagaaccggccgtgttcttggcttgcacacacacacacacacacgcgaaagcagcgcatgcaacaaagcaaagatcacacacggccggagcaagagagacgcccatattcatgtgatcacctggctgacttttgcattggaaagcaacagtgcgagatcaaaagcgaacgcagcacacagcacagcacagcacagggatcgagttgctctactccgtatatgaggtcatgcagtatataagcactcacatgcagctataccttagctagggcacctgtgcaaggggacaggtaggagataaagcaatgtctgctggctagaataaaaggaggccgatatcgtatcagcagagggttccaggggagaaggccgttctgcagggccgccgtaaaaggctgtgtaggaagcagccacagcttctcatatcttcttgttcttcgactacttgcctagctactatatctactagactagccctaggctgcttgctagctagtaggtgtatcgatcgtccgaggtaggaggagacagcaggaagcaggctctccaccatgggcccactgcagaggcgcccaacagcgatggggggcctggaggagatcaagaacgaggccgtcgatctggagaacatccctattgaggaggtgttcgagcagctgaagtgcacccgggagggcctcagctcctcggagggccagcagcgcctggagatcttcggcccaaaccggctggaggagaagaaggagagcaagatcctcaagttcctggggttcatgtggaacccgctgtcctgggtcatggagatggctgccgtgatggcgattgctctggctaatggcgggggcaagccaccggactgggaggatttcgtggggattatcgtcctgctcgtcatcaacagcactatctccttcattgaggagaacaacgctggcaacgccgctgctgcgctgatggcgaacctcgctccaaagactaaggtcctccgcgacggccgctggggggagcaggaggctgcgattctcgtgcccggggacatcgttagcattaagctcggggatatcgtccctgctgatgcccggctgctggagggggaccccctgaaggtcgaccagagcgctctcaccggcgagtcgctcccagtcactaagggccctggggacgaggtgttcagcggctctacgtgcaagcagggcgagatcgaggcggtcgtgatcgctaccggcgtgcacaccttcttcgggaaggccgcccatctcgtggattcgacaaaccaggtcgggcacttccagcaggtgctgacagccatcgggaacttctgcatctgctcgattgccgttgggatcgtggttgagattattgtgatgttcccgattcagcaccggcgctaccgctccgggatcgagaacctcctggtcctcctcattggggggattccgatcgctatgccgaccgtcctgagcgtcactatggctatcgggtctcacaagctctcacagcagggggcgattaccaagcggatgacagcgattgaggagatggcggggatggacgtgctgtgctcagacaagaccggcactctgacgctcaacaagctctccgtggacaagaacctcgtcgaggtgttctgcaagggggtggacaaggatcatgtcctgctgctggcggccagggcctcccggacagagaaccaggacgccattgacgccgctatggtggggatgctggccgaccctaaggaggcgagggccggcatccgcgagattcacttcctgccattcaatcccgttgacaagcgcactgcgctgacgtacattgatgcggacgggcactggcacagggtgtccaagggggctccagagcagatcctcgatctgtgccactgcaaggaggatctgcggcgcaaggtgcatgggatcattgacaagtacgcggagagggggctccggagcctggcggtggccaggcaggaggtgccggagcgcaacaaggagtcgccggggggcccttggcagttcgtcgggctgctccctctgttcgatcccccgaggcacgacagcgcggagactatccgcaaggccctcgttctgggggtgaatgtcaagatgattaccggggaccagctggccattgggaaggagacaggccggcgcctcgggatgggcacgaacatgtacccgtcatccgccctgctggggcagaataaggacgccactctggaggccctgccggtcgatgagctcatcgagaaggctgacgggttcgcgggcgtcttccccgagcataagtacgagatcgtgaagcggctgcaggagaagaagcatattgtggggatgactggcgacggggtgaatgatgctcccgccctgaagaaggctgacatcggcattgccgtcgcggacgccacagatgctgcccgctcggcgtccgatatcgttctcacggagcctgggctgagcgttattatctcggcggttctgacaagcaggtgcatcttccagcgcatgaagaactacaccatctacgctgtgtcgatcactatcaggatcgtgctgggcttcatgctgattgcgctcatctggcagtacgacttctcccctttcatggtcctgatcattgctatcctcaacgacggcacaatcatgactatctcgaaggaccgcgtgaagccgagcccactccccgactcttggaagctcaaggagattttcgccactgggatcgttctcgggtcttacctcgctctgatgaccgtgattttcttctgggctatgcacaagaccgacttcttcagcgacaagttcggggttcgctcgattcgcgactcggagcacgagatgatgtctgcgctgtacctccaggtttcaattgtttcacaggcgctcattttcgtgactaggtcacggtcttggtccttcgtcgagaggcctggcctgctcctggtcactgctttcctgctcgcccagctggtggccactttcctcgctgtttacgctaattgggggttcgctaggatcaaggggatcggctgggggtgggcgggcgtggtctggctctactccatcgttttctacttcccactcgacctcatcaagttcttcattcggttcgtgctgtcagggagggcgtgggacaacctcctggagaacaagaccgcgttcaccactaagaaggactacggccgcgaggagagggaggcgcagctggctacagcgcagcgcaccctccacgggctccagcccccagaggctgcgacatctacactcttccacgacaagaattcttaccgcgagctgtcagagattgccgagcaggctaagcgcagggcggagattgctaggctgagggagctcaacacactgaagggccacgtggagtcagtggtcaagctgaagggcctggacatcgatacaattcagcagaactacaccgtttagaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 62 - DNA, 2796 bp, Arabidopsis thaliana
  misc_feature 1..2796: note = AtAHA5W857L
  source 1..2796: mol_type = other DNA
atggaggaggttttcgaggagctgaagtgcaccaagcaggggctgaccgctaatgaggcgtcccacaggctggatgttttcgggccaaacaagctggaggagaagaaggagtctaagctcctgaagttcctgggcttcatgtggaaccccctctcatgggttatggaggtggctgctctgatggctatcgctctcgccaatggcgggggcaggccgcccgactggcaggatttcgtcggcattgtttgcctcctgctcatcaactctacgatttcattcatcgaggagaacaatgctggcaatgctgccgcggctctgatggccgggctcgcgccaaagacaaaggtgctcagggacaaccagtggtctgagcaggaggcgtcaatcctggtgccaggcgatgtcatctccattaagctcggggacatcattccagctgatgctaggctgctcgacggcgatccactgaagatcgaccagtccagcctcacgggcgagtcgattcccgtcacaaagaacccatctgatgaggttttcagcggctcgatttgcaagcagggggagatcgaggccatcgtcattgcgactggcgttcacaccttcttcgggaaggccgcgcatctggtggacaacactaatcagatcggccacttccagaaggtcctgacctcaattgggaacttctgcatttgctccatcgccctcggcatcattgttgagctgctcgtgatgtacccaatccagaggaggcgctaccgcgacggcattgataatctgctcgtgctgctcatcgggggcatccctattgcgatgccgtccgtgctgagcgtcacgatggctacaggctcccaccgcctcttccagcagggggctattacgaagcgcatgacagctatcgaggagatggccgggatggacgtgctctgctgcgataagacaggcactctgaccctcaacaagctgaccgtcgacaagaatctcgtcgaggttttcgcgaagggggtgggcaaggagcatgtcttcctgctcgctgcccgcgcctcccgcatcgagaaccaggacgccattgatgctgctatcgtcggcatgctggccgacccaaaggaggccagggcgggcgttcgggaggtgcacttcttccctttcaacccggtggacaagcgcactgcgctcacctacgtcgacagcgatggcaattggcatagggcttcgaagggggccccagagcagatcctgaacctctgcaattgcaaggaggacgtgcgcaggaaggtccacggcgttatcgataagttcgctgagagggggctgaggtcgctcgctgttgcccggcaggaggtgctggagaagaagaaggacgctccagggggcccgtggcagctggtcggcctgctccctctcttcgacccacctcgccatgatagcgctgagacgatccggcgcgctctgaacctcggcgtgaatgtcaagatgattacaggggaccagctggccatcggcaaggagacggggaggaggctcgggatgggcacaaacatgtacccgtcgtctgctctgctcggccaggtcaaggactcatccctgggggcgctccctgttgacgagctcatcgagaaggcggatggcttcgctggggttttcccggagcacaagtacgagatcgtgcatcgcctgcagcagaggaaccacatctgcggcatgacgggggacggcgtgaatgatgcgcctgctctcaagaaggccgacatcggcattgccgtggtcgacgctacggatgctgcgaggggggcgagcgatatcgtcctgacagagccgggcctctcagtcatcatttccgccgttctgacctcccgggcgattttccagcgcatgaagaactacactatctacgccgttagcatcaccattcgcatcgtgttcggcttcatgttcattgcgctcatctggcagttcgacttctcgccgttcatggtgctgatcattgccatcctcaatgacggcacgattatgacaatctctaaggataggatgaagccgtccccacagccagacagctggaagctgagggatatcttctccactggcgttgtgctggggggctaccaggctctcatgaccgtcgtgttcttctgggtcatgaaggacagcgacttcttctccaactacttcggcgtcaggcctctgagccagaggccagagcagatgatggctgctctgtacctccaggtgtcgatcatttctcaggccctcatcttcgtcacccggtctcgctcatggtcctacgctgagtgcccaggcctgctcctgctcggggctttcgtgattgcccagctggtcgcgaccttcatcgctgtgtacgccaactggtcattcgcgaggatcgagggggctggctgggggtgggctggcgtcatttggctgtactccttcctcacctacatcccactcgacctgctcaagttcggcattcgctacgtgctgagcgggaaggcctggctcaacctcctggagaataagactgcgttcaccacgaagaaggactacggcaaggaggagcgcgaggctcagctggcggctgcccagaggaccctgcacggcctccagccggcggagaagaacaatatcttcaacgagaagaatagctactcggagctctcgcagatcgctgagcaggctaagcgcagggccgaggtggtcaggctgagggagatcaacacgctcaagggccacgtggagtctgttgtgaagctgaagggcctcgacattgatactatccagcagcattacaccgtctag

SEQ ID NO: 63 - DNA, 4326 bp, synthetic construct
  misc_feature 1..4326: note = Cassette_T02780_CsVMV AtAHA5_W857L
  source 1..4326: mol_type = other DNA
aaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtccctcccccctccccctccgccgccgccgcgccggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgatatctccaccatggaggaggttttcgaggagctgaagtgcaccaagcaggggctgaccgctaatgaggcgtcccacaggctggatgttttcgggccaaacaagctggaggagaagaaggagtctaagctcctgaagttcctgggcttcatgtggaaccccctctcatgggttatggaggtggctgctctgatggctatcgctctcgccaatggcgggggcaggccgcccgactggcaggatttcgtcggcattgtttgcctcctgctcatcaactctacgatttcattcatcgaggagaacaatgctggcaatgctgccgcggctctgatggccgggctcgcgccaaagacaaaggtgctcagggacaaccagtggtctgagcaggaggcgtcaatcctggtgccaggcgatgtcatctccattaagctcggggacatcattccagctgatgctaggctgctcgacggcgatccactgaagatcgaccagtccagcctcacgggcgagtcgattcccgtcacaaagaacccatctgatgaggttttcagcggctcgatttgcaagcagggggagatcgaggccatcgtcattgcgactggcgttcacaccttcttcgggaaggccgcgcatctggtggacaacactaatcagatcggccacttccagaaggtcctgacctcaattgggaacttctgcatttgctccatcgccctcggcatcattgttgagctgctcgtgatgtacccaatccagaggaggcgctaccgcgacggcattgataatctgctcgtgctgctcatcgggggcatccctattgcgatgccgtccgtgctgagcgtcacgatggctacaggctcccaccgcctcttccagcagggggctattacgaagcgcatgacagctatcgaggagatggccgggatggacgtgctctgctgcgataagacaggcactctgaccctcaacaagctgaccgtcgacaagaatctcgtcgaggttttcgcgaagggggtgggcaaggagcatgtcttcctgctcgctgcccgcgcctcccgcatcgagaaccaggacgccattgatgctgctatcgtcggcatgctggccgacccaaaggaggccagggcgggcgttcgggaggtgcacttcttccctttcaacccggtggacaagcgcactgcgctcacctacgtcgacagcgatggcaattggcatagggcttcgaagggggccccagagcagatcctgaacctctgcaattgcaaggaggacgtgcgcaggaaggtccacggcgttatcgataagttcgctgagagggggctgaggtcgctcgctgttgcccggcaggaggtgctggagaagaagaaggacgctccagggggcccgtggcagctggtcggcctgctccctctcttcgacccacctcgccatgatagcgctgagacgatccggcgcgctctgaacctcggcgtgaatgtcaagatgattacaggggaccagctggccatcggcaaggagacggggaggaggctcgggatgggcacaaacatgtacccgtcgtctgctctgctcggccaggtcaaggactcatccctgggggcgctccctgttgacgagctcatcgagaaggcggatggcttcgctggggttttcccggagcacaagtacgagatcgtgcatcgcctgcagcagaggaaccacatctgcggcatgacgggggacggcgtgaatgatgcgcctgctctcaagaaggccgacatcggcattgccgtggtcgacgctacggatgctgcgaggggggcgagcgatatcgtcctgacagagccgggcctctcagtcatcatttccgccgttctgacctcccgggcgattttccagcgcatgaagaactacactatctacgccgttagcatcaccattcgcatcgtgttcggcttcatgttcattgcgctcatctggcagttcgacttctcgccgttcatggtgctgatcattgccatcctcaatgacggcacgattatgacaatctctaaggataggatgaagccgtccccacagccagacagctggaagctgagggatatcttctccactggcgttgtgctggggggctaccaggctctcatgaccgtcgtgttcttctgggtcatgaaggacagcgacttcttctccaactacttcggcgtcaggcctctgagccagaggccagagcagatgatggctgctctgtacctccaggtgtcgatcatttctcaggccctcatcttcgtcacccggtctcgctcatggtcctacgctgagtgcccaggcctgctcctgctcggggctttcgtgattgcccagctggtcgcgaccttcatcgctgtgtacgccaactggtcattcgcgaggatcgagggggctggctgggggtgggctggcgtcatttggctgtactccttcctcacctacatcccactcgacctgctcaagttcggcattcgctacgtgctgagcgggaaggcctggctcaacctcctggagaataagactgcgttcaccacgaagaaggactacggcaaggaggagcgcgaggctcagctggcggctgcccagaggaccctgcacggcctccagccggcggagaagaacaatatcttcaacgagaagaatagctactcggagctctcgcagatcgctgagcaggctaagcgcagggccgaggtggtcaggctgagggagatcaacacgctcaagggccacgtggagtctgttgtgaagctgaagggcctcgacattgatactatccagcagcattacaccgtctagaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg

SEQ ID NO: 64 - DNA, 4312 bp, synthetic construct
  misc_feature 1..4312: note = Cassette_T02783_rbcS_Mut_al AtAHA5_W857L
  source 1..4312: mol_type = other DNA
tccctttaatctggcgctagatctgcatccgcggcttgcaaagataaatggcacatttagtgtgttattttgcaatacctttcatagtagatatccttaaatgcagttttaggcatgtttgggtaattaaataacatttttaggaggagttttagatttacctttctttcgtgatgactgatgacagacgtggggaattcaaatgcaactctagcgaaagttcatatatttttcataaatagctgaggctggggtaattattttttttgtagaaaaatagaataggtggaatggttggggaaggcgtaggcgctcgtggacgacgcccgataaaagacaagaggcggaattgccatgaattcgaggtagctaagtaaggcgcatatatatgccaaaaaattctactgtcactttccaatttcaatgcgctgccaaacaagccatcctggaaactgacttgaattcagcccaattctgtagatccaaacagggccggcgtcagtgcctcaggtgagagagcagcagacgattcaaagagccaaaactgcaagcagacgcagccgaagccgaagccgaagcccaagcccaaaactgttttgtctttgcccagaaccgcgacgagcctaaactgcgcttcctcctatctacaagtccctggcacatcacgcatagtccaacctaggcgcgcaggcgataaggcgcgccacggggacgcgacatgtggtggcggacgcgatcaggatagggccaggctggccgggcgcggccacgggagaacggtggccactcgtcccacatccgcttcgtcctgtcctgtactgcgtcctgcccccaacgagagccggagccggccatcccgtcgcacactctccccctctatatatgccgtcggtgtgggggagcctactacaggacgacccaagcaagcaagcaagcagcgagtacatacatactaggcagccaggcagtctccaccatggaggaggttttcgaggagctgaagtgcaccaagcaggggctgaccgctaatgaggcgtcccacaggctggatgttttcgggccaaacaagctggaggagaagaaggagtctaagctcctgaagttcctgggcttcatgtggaaccccctctcatgggttatggaggtggctgctctgatggctatcgctctcgccaatggcgggggcaggccgcccgactggcaggatttcgtcggcattgtttgcctcctgctcatcaactctacgatttcattcatcgaggagaacaatgctggcaatgctgccgcggctctgatggccgggctcgcgccaaagacaaaggtgctcagggacaaccagtggtctgagcaggaggcgtcaatcctggtgccaggcgatgtcatctccattaagctcggggacatcattccagctgatgctaggctgctcgacggcgatccactgaagatcgaccagtccagcctcacgggcgagtcgattcccgtcacaaagaacccatctgatgaggttttcagcggctcgatttgcaagcagggggagatcgaggccatcgtcattgcgactggcgttcacaccttcttcgggaaggccgcgcatctggtggacaacactaatcagatcggccacttccagaaggtcctgacctcaattgggaacttctgcatttgctccatcgccctcggcatcattgttgagctgctcgtgatgtacccaatccagaggaggcgctaccgcgacggcattgataatctgctcgtgctgctcatcgggggcatccctattgcgatgccgtccgtgctgagcgtcacgatggctacaggctcccaccgcctcttccagcagggggctattacgaagcgcatgacagctatcgaggagatggccgggatggacgtgctctgctgcgataagacaggcactctgaccctcaacaagctgaccgtcgacaagaatctcgtcgaggttttcgcgaagggggtgggcaaggagcatgtcttcctgctcgctgcccgcgcctcccgcatcgagaaccaggacgccattgatgctgctatcgtcggcatgctggccgacccaaaggaggccagggcgggcgttcgggaggtgcacttcttccctttcaacccggtggacaagcgcactgcgctcacctacgtcgacagcgatggcaattggcatagggcttcgaagggggccccagagcagatcctgaacctctgcaattgcaaggaggacgtgcgcaggaaggtccacggcgttatcgataagttcgctgagagggggctgaggtcgctcgctgttgcccggcaggaggtgctggagaagaagaaggacgctccagggggcccgtggcagctggtcggcctgctccctctcttcgacccacctcgccatgatagcgctgagacgatccggcgcgctctgaacctcggcgtgaatgtcaagatgattacaggggaccagctggccatcggcaaggagacggggaggaggctcgggatgggcacaaacatgtacccgtcgtctgctctgctcggccaggtcaaggactcatccctgggggcgctccctgttgacgagctcatcgagaaggcggatggcttcgctggggttttcccggagcacaagtacgagatcgtgcatcgcctgcagcagaggaaccacatctgcggcatgacgggggacggcgtgaatgatgcgcctgctctcaagaaggccgacatcggcattgccgtggtcgacgctacggatgctgcgaggggggcgagcgatatcgtcctgacagagccgggcctctcagtcatcatttccgccgttctgacctcccgggcgattttccagcgcatgaagaactacactatctacgccgttagcatcaccattcgcatcgtgttcggcttcatgttcattgcgctcatctggcagttcgacttctcgccgttcatggtgctgatcattgccatcctcaatgacggcacgattatgacaatctctaaggataggatgaagccgtccccacagccagacagctggaagctgagggatatcttctccactggcgttgtgctggggggctaccaggctctcatgaccgtcgtgttcttctgggtcatgaaggacagcgacttcttctccaactacttcggcgtcaggcctctgagccagaggccagagcagatgatggctgctctgtacctccaggtgtcgatcatttctcaggccctcatcttcgtcacccggtctcgctcatggtcctacgctgagtgcccaggcctgctcctgctcggggctttcgtgattgcccagctggtcgcgaccttcatcgctgtgtacgccaactggtcattcgcgaggatcgagggggctggctgggggtgggctggcgtcatttggctgtactccttcctcacctacatcccactcgacctgctcaagttcggcattcgctacgtgctgagcgggaaggcctggctcaacctcctggagaataagactgcgttcaccacgaagaaggactacggcaaggaggagcgcgaggctcagctggcggctgcccagaggaccctgcacggcctccagccggcggagaagaacaatatcttcaacgagaagaatagctactcggagctctcgcagatcgctgagcaggctaagcgcagggccgaggtggtcaggctgagggagatcaacacgctcaagggccacgtggagtctgttgtgaagctgaagggcctcgacattgatactatccagcagcattacaccgtctagaactagaccgcgcccgccggccgccccccgccggctagctagctagctagctagctcctgcgtgagctagtagctagtgccatgcgtcgtctctgtcgttcggttttgcttcgggtcaccgtaccctttgcttgcttggtttcttctttccttttttccttttttttttcttcttttccccggccatggttcctttgctttcagcagttctctgctggatgtgatgtatccattgttgcaagcatggccttgcattggctacctctatacctgctacaaactactgcaacgcctatatatacttggggtgaggaacatgtgaatgcaagctccggctatcatatacatgtaatatggatacaaactatatatataaatccgccgaggcgccgactaatactatacgacgacaccgtgttaagttaatatataactggtgctttttatttatatatctgtctcatcatatatatatgctaattaatggatgtgtgtcctcttcacttcaattccttctttcctttcctatgctttgagatc

SEQ ID NO: 65 - DNA, 5181 bp, synthetic construct
  misc_feature 1..5181: note = Cassette_T10191_RAB17_del AtAHA5_W857L
  source 1..5181: mol_type = other DNA
aaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcatctctccaccatggaggaggttttcgaggagctgaagtgcaccaagcaggggctgaccgctaatgaggcgtcccacaggctggatgttttcgggccaaacaagctggaggagaagaaggagtctaagctcctgaagttcctgggcttcatgtggaaccccctctcatgggttatggaggtggctgctctgatggctatcgctctcgccaatggcgggggcaggccgcccgactggcaggatttcgtcggcattgtttgcctcctgctcatcaactctacgatttcattcatcgaggagaacaatgctggcaatgctgccgcggctctgatggccgggctcgcgccaaagacaaaggtgctcagggacaaccagtggtctgagcaggaggcgtcaatcctggtgccaggcgatgtcatctccattaagctcggggacatcattccagctgatgctaggctgctcgacggcgatccactgaagatcgaccagtccagcctcacgggcgagtcgattcccgtcacaaagaacccatctgatgaggttttcagcggctcgatttgcaagcagggggagatcgaggccatcgtcattgcgactggcgttcacaccttcttcgggaaggccgcgcatctggtggacaacactaatcagatcggccacttccagaaggtcctgacctcaattgggaacttctgcatttgctccatcgccctcggcatcattgttgagctgctcgtgatgtacccaatccagaggaggcgctaccgcgacggcattgataatctgctcgtgctgctcatcgggggcatccctattgcgatgccgtccgtgctgagcgtcacgatggctacaggctcccaccgcctcttccagcagggggctattacgaagcgcatgacagctatcgaggagatggccgggatggacgtgctctgctgcgataagacaggcactctgaccctcaacaagctgaccgtcgacaagaatctcgtcgaggttttcgcgaagggggtgggcaaggagcatgtcttcctgctcgctgcccgcgcctcccgcatcgagaaccaggacgccattgatgctgctatcgtcggcatgctggccgacccaaaggaggccagggcgggcgttcgggaggtgcacttcttccctttcaacccggtggacaagcgcactgcgctcacctacgtcgacagcgatggcaattggcatagggcttcgaagggggccccagagcagatcctgaacctctgcaattgcaaggaggacgtgcgcaggaaggtccacggcgttatcgataagttcgctgagagggggctgaggtcgctcgctgttgcccggcaggaggtgctggagaagaagaaggacgctccagggggcccgtggcagctggtcggcctgctccctctcttcgacccacctcgccatgatagcgctgagacgatccggcgcgctctgaacctcggcgtgaatgtcaagatgattacaggggaccagctggccatcggcaaggagacggggaggaggctcgggatgggcacaaacatgtacccgtcgtctgctctgctcggccaggtcaaggactcatccctgggggcgctccctgttgacgagctcatcgagaaggcggatggcttcgctggggttttcccggagcacaagtacgagatcgtgcatcgcctgcagcagaggaaccacatctgcggcatgacgggggacggcgtgaatgatgcgcctgctctcaagaaggccgacatcggcattgccgtggtcgacgctacggatgctgcgaggggggcgagcgatatcgtcctgacagagccgggcctctcagtcatcatttccgccgttctgacctcccgggcgattttccagcgcatgaagaactacactatctacgccgttagcatcaccattcgcatcgtgttcggcttcatgttcattgcgctcatctggcagttcgacttctcgccgttcatggtgctgatcattgccatcctcaatgacggcacgattatgacaatctctaaggataggatgaagccgtccccacagccagacagctggaagctgagggatatcttctccactggcgttgtgctggggggctaccaggctctcatgaccgtcgtgttcttctgggtcatgaaggacagcgacttcttctccaactacttcggcgtcaggcctctgagccagaggccagagcagatgatggctgctctgtacctccaggtgtcgatcatttctcaggccctcatcttcgtcacccggtctcgctcatggtcctacgctgagtgcccaggcctgctcctgctcggggctttcgtgattgcccagctggtcgcgaccttcatcgctgtgtacgccaactggtcattcgcgaggatcgagggggctggctgggggtgggctggcgtcatttggctgtactccttcctcacctacatcccactcgacctgctcaagttcggcattcgctacgtgctgagcgggaaggcctggctcaacctcctggagaataagactgcgttcaccacgaagaaggactacggcaaggaggagcgcgaggctcagctggcggctgcccagaggaccctgcacggcctccagccggcggagaagaacaatatcttcaacgagaagaatagctactcggagctctcgcagatcgctgagcaggctaagcgcagggccgaggtggtcaggctgagggagatcaacacgctcaagggccacgtggagtctgttgtgaagctgaagggcctcgacattgatactatccagcagcattacaccgtctagaacgcggcgcctatacgtggctgtgctgtgctgtctggcgcgtcaaagccgtactcttcagcgttccatagataataagataaacccatgaataagtgtccctaccctttgatcatgtgacagggacagggacagtttcctgtacttgtggggtgaatttgtacgtgtgatacggtaacttggtccgtgtactgtttatgctgctacgtttccttcctaaaagtacgtatatatatacagcacttgcgagtggctttacttgtgtgcactgcctaaaagtgtcgatattacctccagcggtaaataaatgacccttgtaagaaacttacacagcatgtcttactaattaacagccgttattatgttcggcgcagtactcacactggaacttacgaacaagaattgctctatcaatcaccatctactttagatatatcttctgaaacttttttcaaatccaaaagttcagtatcttcctacaaattagtgaattaggaatccaagtggttaaacgaattcggccggccggtaccccttaggcctagggctagaggacccagctttgagtggccgtagatttgcaaaagcaatggctaacagacacatattctgccaaaccccaagaaggataatcacttttcttagataaaaaagaacagaccaatatacaaacatccacacttctgcaaacaatacatcagaactaggattacgccgattacgtggctttagcagactgtccaaaaatctgttttgcaaagctccaattgctccttgcttatccagcttcttttgtgttggcaaactgcgcttttccaaccgattttgttcttctcgcgctttcttcttagcctaaacaaacctcaccgtgcacgcagccatggtcctgaaccttcacctcgtccctataaaagcctagccaaccttcacaatcttatcatcacccacaacaccgagcaccacaaactagagatcctctccaccatggctcagtcaaagcacgggctcacaaaggagatgactatgaagtacaggatggagggctgcgtcgatgggcacaagttcgttatcaccggcgaggggatcggctacccgttcaagggcaagcaggcgattaacctgtgcgtggtcgagggcgggccactgcccttcgctgaggatatcctctccgccgcgttcatgtacggcaacagggttttcaccgagtacccgcaggacattgtcgattacttcaagaattcctgcccagctgggtacacgtgggacaggagcttcctcttcgaggatggcgctgtgtgcatctgcaacgccgacattaccgtttcggtggaggagaattgcatgtaccacgagtctaagttctacggggtgaacttcccagctgacggccccgtcatgaagaagatgacggataattgggagccatcatgcgagaagatcattccagtgcctaagcaggggatcctgaagggcgacgtctccatgtacctcctgctcaaggatggcggcaggctcaggtgccagttcgacacagtctacaaggccaagagcgttccacgcaagatgcctgactggcacttcatccagcataagctgactcgggaggaccgctcggatgcgaagaaccagaagtggcacctgactgagcacgctatcgcctcggggtccgccctcccttgaaacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactag

SEQ ID NO: 66 - DNA, 4238 bp, synthetic construct
  misc_feature 1..4238: note = Cassette_T10586_MYB60 AtAHA5_W857L
  source 1..4238: mol_type = other DNA
ctccgtcgctagcgttgtaatttttatcagcctctctccatctgattggatgagctgcttctcctagctagcacgttgtcaaccattggggacgccctttaggagtgaaagtgatgcagttactctactcccatccggtccaaatcccaacctaagggatatatgacgaagagttttcaatttcagtataatgtgtatagcaagtgatttgattttcctgcagattcttgagagagaagggccaaacctgcatatgtgccaggtgcgcactcacagtcacacacacggtcctcatcaatgcccaagaaatctggagagacacagacaaacgcaagcaattactccattcatgagcgccacagcagaaaaaggagtcctccagaaccggccgtgttcttggcttgcacacacacacacacacacgcgaaagcagcgcatgcaacaaagcaaagatcacacacggccggagcaagagagacgcccatattcatgtgatcacctggctgacttttgcattggaaagcaacagtgcgagatcaaaagcgaacgcagcacacagcacagcacagcacagggatcgagttgctctactccgtatatgaggtcatgcagtatataagcactcacatgcagctataccttagctagggcacctgtgcaaggggacaggtaggagataaagcaatgtctgctggctagaataaaaggaggccgatatcgtatcagcagagggttccaggggagaaggccgttctgcagggccgccgtaaaaggctgtgtaggaagcagccacagcttctcatatcttcttgttcttcgactacttgcctagctactatatctactagactagccctaggctgcttgctagctagtaggtgtatcgatcgtccgaggtaggaggagacagcaggaagcaggctctccaccatggaggaggttttcgaggagctgaagtgcaccaagcaggggctgaccgctaatgaggcgtcccacaggctggatgttttcgggccaaacaagctggaggagaagaaggagtctaagctcctgaagttcctgggcttcatgtggaaccccctctcatgggttatggaggtggctgctctgatggctatcgctctcgccaatggcgggggcaggccgcccgactggcaggatttcgtcggcattgtttgcctcctgctcatcaactctacgatttcattcatcgaggagaacaatgctggcaatgctgccgcggctctgatggccgggctcgcgccaaagacaaaggtgctcagggacaaccagtggtctgagcaggaggcgtcaatcctggtgccaggcgatgtcatctccattaagctcggggacatcattccagctgatgctaggctgctcgacggcgatccactgaagatcgaccagtccagcctcacgggcgagtcgattcccgtcacaaagaacccatctgatgaggttttcagcggctcgatttgcaagcagggggagatcgaggccatcgtcattgcgactggcgttcacaccttcttcgggaaggccgcgcatctggtggacaacactaatcagatcggccacttccagaaggtcctgacctcaattgggaacttctgcatttgctccatcgccctcggcatcattgttgagctgctcgtgatgtacccaatccagaggaggcgctaccgcgacggcattgataatctgctcgtgctgctcatcgggggcatccctattgcgatgccgtccgtgctgagcgtcacgatggctacaggctcccaccgcctcttccagcagggggctattacgaagcgcatgacagctatcgaggagatggccgggatggacgtgctctgctgcgataagacaggcactctgaccctcaacaagctgaccgtcgacaagaatctcgtcgaggttttcgcgaagggggtgggcaaggagcatgtcttcctgctcgctgcccgcgcctcccgcatcgagaaccaggacgccattgatgctgctatcgtcggcatgctggccgacccaaaggaggccagggcgggcgttcgggaggtgcacttcttccctttcaacccggtggacaagcgcactgcgctcacctacgtcgacagcgatggcaattggcatagggcttcgaagggggccccagagcagatcctgaacctctgcaattgcaaggaggacgtgcgcaggaaggtccacggcgttatcgataagttcgctgagagggggctgaggtcgctcgctgttgcccggcaggaggtgctggagaagaagaaggacgctccagggggcccgtggcagctggtcggcctgctccctctcttcgacccacctcgccatgatagcgctgagacgatccggcgcgctctgaacctcggcgtgaatgtcaagatgattacaggggaccagctggccatcggcaaggagacggggaggaggctcgggatgggcacaaacatgtacccgtcgtctgctctgctcggccaggtcaaggactcatccctgggggcgctccctgttgacgagctcatcgagaaggcggatggcttcgctggggttttcccggagcacaagtacgagatcgtgcatcgcctgcagcagaggaaccacatctgcggcatgacgggggacggcgtgaatgatgcgcctgctctcaagaaggccgacatcggcattgccgtggtcgacgctacggatgctgcgaggggggcgagcgatatcgtcctgacagagccgggcctctcagtcatcatttccgccgttctgacctcccgggcgattttccagcgcatgaagaactacactatctacgccgttagcatcaccattcgcatcgtgttcggcttcatgttcattgcgctcatctggcagttcgacttctcgccgttcatggtgctgatcattgccatcctcaatgacggcacgattatgacaatctctaaggataggatgaagccgtccccacagccagacagctggaagctgagggatatcttctccactggcgttgtgctggggggctaccaggctctcatgaccgtcgtgttcttctgggtcatgaaggacagcgacttcttctccaactacttcggcgtcaggcctctgagccagaggccagagcagatgatggctgctctgtacctccaggtgtcgatcatttctcaggccctcatcttcgtcacccggtctcgctcatggtcctacgctgagtgcccaggcctgctcctgctcggggctttcgtgattgcccagctggtcgcgaccttcatcgctgtgtacgccaactggtcattcgcgaggatcgagggggctggctgggggtgggctggcgtcatttggctgtactccttcctcacctacatcccactcgacctgctcaagttcggcattcgctacgtgctgagcgggaaggcctggctcaacctcctggagaataagactgcgttcaccacgaagaaggactacggcaaggaggagcgcgaggctcagctggcggctgcccagaggaccctgcacggcctccagccggcggagaagaacaatatcttcaacgagaagaatagctactcggagctctcgcagatcgctgagcaggctaagcgcagggccgaggtggtcaggctgagggagatcaacacgctcaagggccacgtggagtctgttgtgaagctgaagggcctcgacattgatactatccagcagcattacaccgtctagaacctaaatgctcttaactgagctaattatgtaatgcacatacacatatttacatagatatgcatatttatatatagcatgtatattgtactacatgcattgcttcttaatacatgtagtaaagatatatgcaaaaatagtcgaaagatttgtttacatataaaatcaccaatatttattgttattgtattttcatgaataaagtaataagattatttgtctaatattttgatttactagtactagaaatgaaaaggaatatgcacaatttcagcattatagtttggtaggcaaaatggagtgagaatagagtttcatagtatatactaaggttcttaattgtgcaaatagttgatacaagtcacatgggccaagtttgtaaatcttaaatcgaaatatgccttcttctttttttgcatgaaaatgctagtaatttataagtgtgtttttcaataagagatgctaaataccaaaattaacctagttttcagtgagcgcttgcattattgtgg